(19) United States
(12) Patent Application Publication
(10) Pub. No.: US 2023/0172869 A1
(43) Pub. Date: Jun. 8, 2023

(54) A NANO-ENABLED VACCINATION APPROACH FOR CORONAVIRUS DISEASE (COVID-19) AND OTHER VIRAL DISEASES

(71) Applicant: The Regents of the University of California, Oakland, CA (US)

(72) Inventors: Andre E. Nel, Sherman Oaks, CA (US); Huan Meng, Los Angeles, CA (US); Tian Xia, Los Angeles, CA (US); Qi Liu, Los Angeles, CA (US); Sean Allen, Culver City, CA (US)

(73) Assignee: The Regents of the University of California, Oakland, CA (US)

(21) Appl. No.: 17/919,734

(22) PCT Filed: Apr. 19, 2021
(86) PCT No.: PCT/US2021/028004

§ 371 Date: Oct. 18, 2022

Related U.S. Application Data
(60) Provisional application No. 62/706,567, filed on Aug. 25, 2020.
(60) Provisional application No. 63/027,897, filed on May 20, 2020.
(60) Provisional application No. 63/012,791, filed on Apr. 20, 2020.

(51) Int. Cl. A61K 9/51; A61K 47/69; A61K 39/215; A61K 39/39; C07K 14/005; A61P 31/14

(52) U.S. Cl. CPC A61K 9/5153; A61K 47/6937; A61K 39/215; A61K 39/39; C07K 14/005; A61P 31/14; A61K 2039/572

(57) ABSTRACT

In various embodiments immunogenic nanoparticles are provided that are capable of raising an immune response directed against one or more viral proteins and/or protein fragments. In certain embodiments the immunogenic nanoparticles raise an immune response directed against a virus (e.g., SARS-CoV-2 (2019-nCoV), SARS-CoV-2, and MERS-CoV, and the like). In certain embodiments the immunogenic nanoparticles comprise a nanoparticle formed from one or more biocompatible polymer(s), one or more viral proteins or fragments thereof encapsulated within or attached to the biocompatible polymer(s) where the viral protein or fragment thereof comprises an antigen to which an immune response is to be induced by administration of the immunogenic nanoparticle to a mammal and where the immunogenic nanoparticle further comprises an adjuvant (e.g., a STING agonist).

### CROSS-REFERENCE TO RELATED APPLICATIONS

This application claims priority to and benefit of U.S. Ser. No. 62/706,567, filed on Aug. 25, 2020, U.S. Ser. No. 63/027,897, filed on May 20, 2020, and U.S. Ser. No. 63/012,791, filed on Apr. 20, 2020, all of which are incorporated herein by reference in their entirety for all purposes.

### STATEMENT OF GOVERNMENTAL SUPPORT

This invention was made with government support under Grant Numbers CA198846 and ES027237, awarded by the National Institutes of Health. The government has certain rights in the invention.

### INCORPORATION BY REFERENCE OF SEQUENCE LISTING PROVIDED AS A TEXT FILE

[Not Applicable]

### BACKGROUND

The disease now called COVID-19 was first identified in December 2019 in a cluster of cases of viral pneumonia linked to a wet market in Wuhan (Zhu et al. (2020) N. Engl. J. Med. 382: 727-733). SARS-CoV-2 infection spreads efficiently between people with a basic reproductive number in the range 2.0 to 2.5 in Wuhan (Li et al. (220) N. Engl. J. Med. 382: 1199-1207; Wu et al. (2020) Lancet, 395: 689-697; Riou & Althaus (2020) Euro. Surveill. doi: 10.2807/1560-7917.ES.2020.25.4.2000058). The modes of transmission have not been determined, but infection is presumed to spread mainly through respiratory droplets and fomites, similar to other respiratory viruses. The World Health Organization declared COVID-19 a global pandemic on 11 Mar. 2020.

Since its emergence in late 2019, the novel beta coronavirus SARS-CoV-2 virus has infected over 400,000 people worldwide as of Mar. 24, 2020 (//coronavirus.jhu.edu/map.html). SARS-CoV-2 is the causative agent of COVID-19, a lower respiratory infection with a global fatality rate of about 4.4% and a hospitalization rate of nearly 20% (//www.ncbi.nlm.nih.gov/pubmed/32064853). Given its propensity for asymptomatic and early symptomatic spread (see, e.g., He et al (2020) medRxiv //doi.org/10.1101/2020.03.15.20036707) SARS-CoV-2 is expected to become endemic, causing repeated outbreaks in the future (see, e.g., Neher et al. (2020) medRxiv //doi.org/10.1101/2020.02.13.20022806). Due to the lack of preexisting human immunity, the virus's rapid spread, and COVID-19's relatively high hospitalization and fatality rate, the development of an effective vaccine against SARS-CoV-2 is of vital public health importance.

### SUMMARY

Various embodiments presented herein may include, but need not be limited to, one or more of the following:

Embodiment 1: An immunogenic nanoparticle comprising:
a nanoparticle comprising a biocompatible polymer;
one or more viral proteins or fragment(s) thereof encapsulated within or attached to said nanoparticle where the viral protein(s) or fragment(s) thereof comprises an antigen to which an immune response is to be induced by administration of said immunogenic nanoparticle to a mammal; and
an adjuvant.

Embodiment 2: The immunogenic nanoparticle of embodiment 1, wherein said one or more proteins or fragments are conjugated to, or co-encapsulated with, said adjuvant.

Embodiment 3: An immunogenic nanoparticle comprising:
a nanoparticle comprising a biocompatible polymer; and
a nucleic encapsulated within or attached to said nanoparticle where said nucleic acid encodes one or more viral protein(s) or fragment(s) thereof that comprise one or more antigen(s) to which an immune response is to be induced by administration of said immunogenic nanoparticle to a mammal.

Embodiment 4: The immunogenic nanoparticle of embodiment 3, wherein said nanoparticle further comprises an adjuvant.

Embodiment 5: The immunogenic nanoparticle of embodiment 4, wherein said adjuvant is conjugated to said nucleic acid.

Embodiment 6: The immunogenic particle according to any one of embodiments 3-5, wherein said nucleic acid comprises an mRNA.

Embodiment 7: The immunogenic nanoparticle of embodiment 6, wherein said nucleic acid comprises an mRNA comprising a cap, 5′ and 3′ untranslated regions (UTRs), an open-reading frame (ORF) that encodes said one or more viral protein(s) or fragment(s) thereof that comprise one or more antigen(s) to which an immune response is to be induced, and a 3′ poly(A) tail.

Embodiment 8: The immunogenic nanoparticle of embodiment 7, wherein said nucleic acid further comprises replication machinery derived from a positive-stranded mRNA virus.

Embodiment 9: The immunogenic nanoparticle of embodiment 8, wherein said nucleic acid further comprises replication machinery derived from an alphaviruses such as Sindbis or Semliki-Forest viruses.

Embodiment 10: The immunogenic nanoparticle according to any one of embodiments 1-9, wherein said biocompatible polymer comprises one or more polymers selected from the group consisting of poly(lactic-co-glycolic acid) (PLGA), poly(glycolic acid) (PGA), poly(lactic acid) (PLA), poly(caprolactone) (PCL), poly(butylene succinate), poly(trimethylene carbonate), poly(p-dioxanone), Poly(butylene terephthalate), poly(ester amide) (HYBRANE®), polyurethane, poly[(carboxyphenoxy) propane-sebacic acid], poly[bis(hydroxyethyl) terephthalate-ethyl orthophosphorylate/terephthaloyl chloride], poly(β-hydroxyalkanoate), poly(hydroxybutyrate), poly(hydroxybutyrate-co-hydroxyvalerate), Poly-L-lysine (PLL), poly-ethylenimine (PEI), poly[(2-dimethylamino)ethyl methacrylate (pDMAEMA), polyamidoamine (PAMAM) dendrimers, biodegradable poly(β-amino ester) (PBAE) polymer, and poly(amino-co-ester) (PACE) polymers.

Embodiment 11: The immunogenic nanoparticle according to any one of embodiments 3-9, wherein said biocompatible polymer comprises a cationic polymer.

Embodiment 12: The immunogenic nanoparticle of embodiment 11, wherein said biocompatible polymer comprises a cationic polymer selected from the group consisting of Poly-L-lysine (PLL), poly-ethylenimine (PEI), poly[(2-dimethylamino)ethyl methacrylate (pDMAEMA), polyamidoamine (PAMAM) dendrimers, biodegradable poly(β-amino ester) (PBAE) polymer, and poly(amino-co-ester) (PACE) polymer.

Embodiment 13: The immunogenic nanoparticle according to any one of embodiments 1-9, wherein said biocompatible polymer comprises poly(lactic-co-glycolic acid) (PLGA).

Embodiment 14: The immunogenic nanoparticle of embodiment 13, wherein said PLGA comprises a lactide/glycolid molar ratio ranging from about 30:70 to about 70:30.

Embodiment 15: The immunogenic nanoparticle of embodiment 14, wherein said PLGA comprises a lactide/glycolid molar ratio of about 50:50.

Embodiment 16: The immunogenic nanoparticle according to any one of embodiments 1-15, wherein said nanoparticles are of a size effective for phagocytic uptake by macrophages and/or dendritic cells.

Embodiment 17: The immunogenic nanoparticle of embodiment 16, wherein said nanoparticle ranges in size from about 50 nm up to about 3 μm.

Embodiment 18: The immunogenic nanoparticle of embodiment 17, wherein said nanoparticle ranges in size from about 50 nm, or from about 75 nm, or from about 100 nm, or from about 125 nm, or from about 150 nm, or from about 200 nm, or from about 250 nm, or from about 300 nm, or from about 350 nm, or from about 400 nm, or from about 450 nm, or from about 500 nm up to about 3 μm, or up to about 2.75 μm, or up to about 2.5 μm, or up to about 2.0 μm, or up to about 1.75 μm, or up to about 1.50 μm, or up to about 1.25 μm, or up to about 1 μm, or up to about 900 nm, or up to about 800 nm.

Embodiment 19: The immunogenic nanoparticle of embodiment 18, wherein said nanoparticle ranges in size from 500 to 800 nm.

Embodiment 20: The immunogenic nanoparticle according to any one of embodiments 1-19, wherein the material forming said nanoparticle consists entirely of said biocompatible polymer and said viral proteins or fragment(s) thereof and/or said nucleic acid.

Embodiment 21: The immunogenic nanoparticle according to any one of embodiments 1-19, wherein the material forming said nanoparticle consists of a biocompatible PLGA polymer that is coated with chitosan (a linear polysaccharide, composed of randomly distributed D-glucosamine plus N-acetyl-D-glucosamine), to provide a particle surface that allows nucleic acid binding.

Embodiment 22: The immunogenic nanoparticle according to any one of embodiments 1-19, wherein said nanoparticle additionally comprises a lipid.

Embodiment 23: The immunogenic nanoparticle of embodiment 22, wherein said lipid is a lipid capable of activating a danger signal and/or binding a nucleic acid.

Embodiment 24: The immunogenic nanoparticle of embodiment 23, wherein said lipid comprises a lipid selected from the group consisting of didodecyldimethylammonium bromide (DDAB), and 1,2-dioleoyloxy-3-trimethylammonium propane chloride (DOTAP), DOTMA (N-[1-(2,3-dioleyloxy)propyl]-N,N,N-trimethylammonium chloride), 1,2-dilinoleyloxy-3-dimethylaminopropane (DLinDMA), dilinoleylmethyl-4 dimethyl aminobutyrate (DLin-MC3-DMA), C12-200 (Lipid 5), 3-(dimethylamino) propyl(12Z,15Z)-3-[(9Z,12Z)-octadeca-9,12-dien-1-yl]henicosa-12,15-dienoate (DMAP-BLP), and (2Z)-non-2-en-1-yl 10-[(Z)-(1-methylpiperidin-4 yl)carbonyloxy]nonadecanoate (L101).

Embodiment 25: The immunogenic nanoparticle according to any one of embodiments 22-24, wherein said lipid comprises up to 25%, (molar percentage) of said nanoparticle.

Embodiment 26: The immunogenic nanoparticle of embodiment 25, wherein said lipid comprises about 1%, or about 2%, or about 3%, or about 4%, or about 5%, or about 6%, or about 7% up to about 25%, or up to about 20%, or up to about 15%, or up to about 10% (molar percentage) of said nanoparticle.

Embodiment 27: The immunogenic nanoparticle according to any one of embodiments 1-26, wherein said one or more viral proteins or fragment(s) thereof are encapsulated within said nanoparticle (e.g., incorporated in said biocompatible polymer).

Embodiment 28: The immunogenic nanoparticle according to any one of embodiments 3-27, wherein said nucleic acid(s) encoding one or more viral proteins or fragment(s) thereof are encapsulated within said nanoparticle (e.g., incorporated in said biocompatible polymer).

Embodiment 29: The immunogenic nanoparticle according to any one of embodiments 1-28, wherein said one or more viral proteins or fragment(s) thereof are attached to the surface of said nanoparticle.

Embodiment 30: The immunogenic nanoparticle according to any one of embodiments 3-29, wherein said nucleic acid(s) encoding one or more viral proteins or fragment(s) thereof are attached to the surface of said nanoparticle.

Embodiment 31: The immunogenic nanoparticle of embodiment 29 or 30, wherein said one or nucleic acids or said one or more viral proteins or fragment(s) thereof are directly attached to the surface of said nanoparticle.

Embodiment 32: The immunogenic nanoparticle of embodiment 31, wherein said one or more nucleic acids or said one or more viral proteins or fragment(s) thereof are attached to the surface of said nanoparticle with a linker.

Embodiment 33: The immunogenic nanoparticle of embodiment 32, wherein said linker comprises a DSPE-PEG-maleimide where said linker is incorporated in or on said biocompatible polymer.

Embodiment 34: The immunogenic nanoparticle according to any one of embodiments 1-33, wherein said one or more viral proteins or fragments thereof are derived from a virus selected from the group consisting of corona viruses (including, but not limited to 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), SARS-CoV-2 (the novel coronavirus that causes coronavirus disease 2019, or COVID-19), and the like), human influenza viruses Types A and B, human Rhinovirus, respiratory syncytial virus (e.g., sin-SISH-uhl), parainfluenza viruses (HPIVs), measles virus, rubella virus, chickenpox/shingles virus, smallpox, chikungunya virus, hepatitis viruses, herpes viruses, human papilloma viruses (HPVs), polio virus, Norovirus, Rotavirus, Marburg virus, Ebola virus, Rabies, Smallpox, HIV, Hantavirus, Dengue, Molluscum contagiosum, Herpes simplex virus-1 (HSV-1), and Varicella-zoster virus (VZV).

Embodiment 35: The immunogenic nanoparticle according to any one of embodiments 1-19, wherein said one or more viral proteins or fragments thereof are derived from a virus weaponized or studied as an agent for biological warfare.

Embodiment 36: The immunogenic nanoparticle of embodiment 35, wherein said virus is selected from the group consisting of the Bunyaviridae (especially Rift Valley fever virus), Ebolavirus, many of the Flaviviridae (especially Japanese encephalitis virus), Machupo virus, Marburg virus, Variola virus, yellow fever virus, viral encephalitis alphaviruses (e.g., venezuelan equine encephalitis, eastern equine encephalitis, western equine encephalitis), hantaviruses, Nipah virus, Tickborne encephalitis viruses, tickborne hemorrhagic fever viruses, and Yellow fever.

Embodiment 37: The immunogenic nanoparticle of embodiment 34, wherein said corona virus comprise a member of the Betacoronavirus Genus.

Embodiment 38: The immunogenic nanoparticle of embodiment 37, wherein said corona virus comprises a corona virus that produces severe acute respiratory syndrome (SARS).

Embodiment 39: The immunogenic nanoparticle of embodiment 38, said corona virus comprises a corona virus selected from the group consisting of that SARS-CoV-2 (2019-nCoV), SARS-CoV-2, and MERS-CoV.

Embodiment 40: The immunogenic nanoparticle of embodiment 39, wherein said corona virus is SARS-CoV-2 (2019-nCoV).

Embodiment 41: The immunogenic nanoparticle according to any one of embodiments 34-40, wherein said one or more viral proteins or fragments thereof comprise a corona virus protein or fragment thereof.

Embodiment 42: The immunogenic nanoparticle of embodiment 41, wherein said one or more viral proteins or fragments thereof comprise a corona virus protein or fragment thereof from a coronavirus that is a member of the Betacoronavirus genus.

Embodiment 43: The immunogenic nanoparticle according to any one of embodiments 41-42, wherein said one or more viral proteins or fragments thereof comprise an N-protein or fragment thereof, an M-protein or fragment thereof, and/or an S-protein or fragment thereof.

Embodiment 44: The immunogenic nanoparticle of embodiment 43, wherein said viral protein(s) or fragment(s) thereof comprise a full-length S-protein.

Embodiment 45: The immunogenic nanoparticle of embodiment 43, wherein said viral protein(s) or fragment(s) thereof comprise a full length S1 subunit of an S-protein.

Embodiment 46: The immunogenic nanoparticle of embodiment 43, wherein said viral protein(s) or fragment(s) thereof comprise a full length S2 subunit of an S-protein.

Embodiment 47: The immunogenic nanoparticle of embodiment 43, wherein said viral proteins or fragments thereof comprises an S protein or S protein fragment comprising a mutation selected from the group consisting of D614G, L452R, E484Q. and E484K.

Embodiment 48: The immunogenic nanoparticle of embodiment 47, wherein said viral proteins or fragments thereof comprises an S protein or S protein fragment comprising a D614G mutation.

Embodiment 49: The immunogenic nanoparticle of according to any one of embodiments 47-48, wherein said viral proteins or fragments thereof comprises an S protein or S protein fragment comprising an L452R mutation.

Embodiment 50: The immunogenic nanoparticle of according to any one of embodiments 47-49, wherein said viral proteins or fragments thereof comprises an S protein or S protein fragment comprising an E484Q mutation.

Embodiment 51: The immunogenic nanoparticle of according to any one of embodiments 47-50, wherein said viral proteins or fragments thereof comprises an S protein or S protein fragment comprising an E484K mutation.

Embodiment 52: The immunogenic nanoparticle according to any one of embodiments 43-51, wherein said viral protein(s) or fragment(s) thereof comprise a full-length N-protein.

Embodiment 53: The immunogenic nanoparticle according to any one of embodiments 43-51, wherein said viral protein(s) or fragment(s) thereof comprise a fragment of an N-protein.

Embodiment 54: The immunogenic nanoparticle according to any one of embodiments 43-53, wherein said viral protein(s) or fragment(s) thereof comprise a T cell epitope from a SARS-CoV-2 S-protein.

Embodiment 55: The immunogenic nanoparticle of embodiment 54, wherein said viral protein(s) or fragment(s) thereof comprise a T cell epitope from a SARS-CoV-2 S-protein shown in Table 1, or an immunogenic fragment thereof.

Embodiment 56: The immunogenic nanoparticle of embodiment 54, wherein said viral protein(s) or fragment(s) thereof comprise a T cell epitope of the SARS-CoV-2 S-protein comprising or consisting of the sequence PAQEKNFTTAPAICH (SEQ ID NO:1), or an immunogenic fragment thereof.

Embodiment 57: The immunogenic nanoparticle of embodiment 54, wherein said viral protein(s) or fragment(s) thereof comprise a T cell epitope of the SARS-CoV-2 S-protein comprising or consisting of the sequence GYHLMSFPQSAPHGV (SEQ ID NO:2), or an immunogenic fragment thereof.

Embodiment 58: The immunogenic nanoparticle of embodiment 54, wherein said viral protein(s) or fragment(s) thereof comprise a T cell epitope of the SARS-CoV-2 S-protein comprising or consisting of the sequence ARSVASQSIIAYTMS (SEQ ID NO:3), or an immunogenic fragment thereof.

Embodiment 59: The immunogenic nanoparticle of embodiment 54, wherein said viral protein(s) or fragment(s) thereof comprise a T cell epitope of the SARS-CoV-2 S-protein comprising or consisting of the sequence IDGYFKIYSKHTPIN (SEQ ID NO:4), or an immunogenic fragment thereof.

Embodiment 60: The immunogenic nanoparticle of embodiment 54, wherein said viral protein(s) or fragment(s) thereof comprise a T cell epitope of the SARS-CoV-2 S-protein comprising or consisting of the sequence QMAYRFNGIGVTQNV (SEQ ID NO:5), or an immunogenic fragment thereof.

Embodiment 61: The immunogenic nanoparticle according to any one of embodiments 43-60, wherein said viral protein(s) or fragment(s) thereof comprise one or more T cell epitopes from a SARS-CoV-2 or SARS-COV N-protein shown in Table 2 (SEQ ID NOs:6-21), or an immunogenic fragment thereof.

Embodiment 62: The immunogenic nanoparticle according to any one of embodiments 43-61, wherein said viral protein(s) or fragment(s) thereof comprise one or more T cell epitopes from a SARS-CoV-2 or SARS-CoV S-protein shown in Table 2 (SEQ ID NOs:22-32), or an immunogenic fragment thereof.

Embodiment 63: The immunogenic nanoparticle according to any one of embodiments 43-62, wherein said viral protein(s) or fragment(s) thereof comprise one or more T cell epitopes from a SARS-CoV-2 or SARS-CoV N-protein and/or S-protein shown in Table 3 (SEQ ID NOs:33-208), or an immunogenic fragment thereof.

Embodiment 64: The immunogenic nanoparticle according to any one of embodiments 43-63, wherein said viral protein(s) or fragment(s) thereof comprise an epitope located in the SARS-CoV receptor-binding motif.

Embodiment 65: The immunogenic nanoparticle of embodiment 64, wherein said viral protein(s) or fragment(s) thereof comprise an epitope comprising or consisting of the a sequence selected from the group consisting of QPYRVVVLSF (SEQ ID NO:643), GYQPYRVVVL (SEQ ID NO:59), and PYRVVVLSF (SEQ ID NO:60), or an immunogenic fragment thereof.

Embodiment 66: The immunogenic nanoparticle according to any one of embodiments 43-65, wherein said viral protein(s) or fragment(s) thereof comprise a SARS-CoV-2 and/or SARS-CoV B cell epitope.

Embodiment 67: The immunogenic nanoparticle of embodiment 66, wherein said viral protein(s) or fragment(s) thereof comprise or consist of a B cell S-protein epitope shown in Table 4 (SEQ ID NOs: 210-232).

Embodiment 68: The immunogenic nanoparticle according to any one of embodiments 66-67, wherein said viral protein(s) or fragment(s) thereof comprise or consist of a B cell N-protein epitope shown in Table 4 (SEQ ID NOs: 233-254).

Embodiment 69: The immunogenic nanoparticle according to any one of embodiments 41-68, wherein said viral protein(s) or fragments thereof comprise or consist of a SARS-CoV B cell epitope shown in Table 5, or an immunogenic fragment thereof.

Embodiment 70: The immunogenic nanoparticle according to any one of embodiments 41-69, wherein said viral protein(s) or fragments thereof comprise or consist of a SARS-CoV T cell epitope shown in Table 6, or an immunogenic fragment thereof.

Embodiment 71: The immunogenic nanoparticle according to any one of embodiments 41-70, wherein said viral protein(s) or fragments thereof comprise or consist of a SARS-CoV-2 B cell epitope shown in Table 5, or an immunogenic fragment thereof.

Embodiment 72: The immunogenic nanoparticle according to any one of embodiments 41-71, wherein said viral protein(s) or fragments thereof comprise or consist of a SARS-CoV-2 T cell epitope shown in Table 6, or an immunogenic fragment thereof.

Embodiment 73: The immunogenic nanoparticle according to any one of embodiments 34-72, wherein said one or more viral proteins or fragments thereof comprise or consist of an amino acid sequence shown in one or more of Tables 10-12.

Embodiment 74: The immunogenic nanoparticle according to any one of embodiments 34-73, wherein said one or more viral proteins or fragments thereof comprise or consist of an amino acid sequence shown in shown in Table 7 and/or in Table 8.

Embodiment 75: The immunogenic nanoparticle of embodiment 74, wherein said one or more viral proteins or fragments thereof comprise or consist of an amino and amino acid sequence shown in Table 7 and an amino acid sequence shown in Table 8.

Embodiment 76: The immunogenic nanoparticle according to any one of embodiments 34-75, wherein said one or more viral proteins or fragments thereof comprise or consist of an amino acid sequence shown in shown in Table 9.

Embodiment 77: The immunogenic nanoparticle according to any one of embodiments 1-79, wherein said adjuvant comprises an adjuvant that elicits a TH1-biased immune response.

Embodiment 78: The immunogenic nanoparticle of embodiment 77, wherein said adjuvant is selected from the group consisting of a combined aluminum salt and TLR4 agonist, rOv-ASP-1 (recombinant Onchocerca volvulus activation associated protein-1, IC31® (a two-component adjuvant consisting of the artificial antimicrobial cationic peptide KLK acting as a vehicle and the TLR9-stimulatory oligodeoxynucleotide ODN1, SPO1, CPG oligonucleotide, alum-TLR7 agonist based on a TLR7 agonist (SMIP7.10), OprI lipoprotein of Pseudomonas aeruginosa, cathelicidin-derived antimicrobial peptides, delta inulin (β-D-[2-1]poly(fructo-furanosyl)α-D-glucose), β-defensin, and a STING agonist.

Embodiment 79: The immunogenic nanoparticle of embodiment 78, wherein said adjuvant comprises one or more STING agonists.

Embodiment 80: The immunogenic nanoparticle of embodiment 79, wherein said adjuvant comprises one or more STING agonists selected from the group consisting of amidobenzimidazole (diABZI), 3′,5′-Cyclic diadenylic acid sodium salt (c-DI-AMP sodium salt), 3′,5′-Cyclic diguanylic acid sodium salt (c-Di-GMP sodium salt), 2′,3′-Cyclic guanosine monophosphate-adenosine monophosphate sodium salt (2′,3′-cGAMP), 3′,3′-Cyclic guanosine monophosphate-adenosine monophosphate sodium salt (3′,3′-cGAMP), 5,6-Dimethyl-9-oxo-9H-xanthene-4-acetic acid (DMXAA), CMA, MK-1454, CRD5500, cyclic di-nucleotide compounds as described in U.S. Patent Publication No: 2020/0062798 A1, tricyclic heteroaryl compounds as described in U.S. Patent Publication No: 2020/0040009 A1, and heteroaryl amide compounds as described in U.S. Patent Publication No: 2020/0039994 A1.

Embodiment 81: The immunogenic nanoparticle of embodiment 80, wherein said adjuvant comprises amidobenzimidazole (diABZI).

Embodiment 82: The immunogenic nanoparticle according to any one of embodiments 1-81, wherein said adjuvant comprises one or more adjuvants selected from the group consisting of CpG ODNs (TLR9 agonists), imiquimod-family compounds (TLR7 agonists), lipopolysaccharide-based compounds (TLR4) LPS-like compounds, Flageline, dsRNA-like compounds (e.g., Poly (I:C) and derivatives), poly (I:C) and derivatives, MatrixM™⁹⁰, AS03, CpG 1018), and Advax.

Embodiment 83: The immunogenic nanoparticle according to any one of embodiments 1-82, wherein said nanoparticles have one or more targeting moieties attached to the surface where said targeting moieties bind to and/or facilitate uptake by antigen presenting cells (APCs).

Embodiment 84: The immunogenic nanoparticle of embodiment 83, wherein said antigen presenting cells comprise one or more cells selected from the group consisting of macrophages, dendritic cells, and B cells.

Embodiment 85: The immunogenic nanoparticle according to any one of embodiments 83-84, wherein said targeting moiety binds to a receptor on a dendritic cell.

Embodiment 86: The immunogenic nanoparticle of embodiment 85, wherein said receptor on a dendritic cell is selected from the group consisting of DEC205, MR, Dectin-1, DC-SIGN, DNGR-1, and FcγR.

Embodiment 87: The immunogenic nanoparticle according to any one of embodiments 85-86, wherein said targeting moiety is selected from the group consisting of SAG-1 (T. gondii), HIV-1 gagP24, Ova, AHc, RSV fusion protein, MUC1, MAA, hCGβ, Ova, Diphtheria toxin (CRM₁₉₇), Ag85B (Mtb), triMN-LPR, Ova, Anti-Clec9A, Ova, MUC1, MUC1-Tn, E75 (HER-2), iFT, gp120_{αga1}/p24, and α-gal.

Embodiment 88: The immunogenic nanoparticle according to any one of embodiments 83-87, wherein said targeting moiety binds to a receptor on a macrophage.

Embodiment 89: The immunogenic nanoparticle of embodiment 88, wherein said targeting moiety binds to a receptor selected from the group consisting of a sialoadhesin receptor, a folate receptor, a galactose receptor, a mannose receptor, a β-glucan receptor, a scavenger receptor, and a tuftsin receptor.

Embodiment 90: The immunogenic nanoparticle of embodiment 89, wherein said targeting moiety is selected from the group consisting of sialic acid, 9-N-(4H-thieno[3,2-c]chromene-2-carbamoyl)-Neu5Acα2-3Ga1β1-4GlcNAc (TCCNeu5Ac), folic acid, methotrexate, folate, galactose residue, lactose, low density lipoprotein (LDL), ovalbumin (OVA), lactobionic acid, mannose-rich glycoconjugates, mannose, mannan, mannosylated poly(L-lysine) (MPL), zymosan and other β-glucans, glucan, poly-guanine, apoB protein fragment, and Tufsin tetrapeptide (Thr-Lys-Pro-Arg, (SEQ ID NO:644)).

Embodiment 91: The immunogenic nanoparticle of embodiment 89, wherein said targeting moiety binds to or more scavenger receptors selected from the group consisting of Stabilin 1, Stabilin 2, and mannose receptor.

Embodiment 92: The immunogenic nanoparticle of embodiment 91, wherein said targeting moiety comprises a fragment of apolipoprotein B protein effective to bind to Stabilin 1 and/or Stabilin 2.

Embodiment 93: The immunogenic nanoparticle of embodiment 92, wherein said targeting moiety fragment ranges in length from about 5, or from about 8, or from about 10 up to about 50, or up to about 40, or up to about 30, or up to about 20 amino acids.

Embodiment 94: The immunogenic nanoparticle of embodiment 93, wherein said targeting moiety comprises a fragment of the apoB protein comprising the amino acid sequence RKRGLK (SEQ ID NO:640).

Embodiment 95: The immunogenic nanoparticle of embodiment 94, wherein said first targeting moiety comprises a fragment of the apoB protein comprising the amino acid sequence RLYRKRGLK (SEQ ID NO:641).

Embodiment 96: The immunogenic nanoparticle of embodiment 94, wherein said first targeting moiety comprise or consists of the amino acid sequence CGGKLGRKYRYLR (SEQ ID NO:642).

Embodiment 97: The immunogenic nanoparticle of embodiment 89, wherein said targeting moiety binds to a mannose receptor.

Embodiment 98: The immunogenic nanoparticle of embodiment 97, wherein said targeting moiety comprises mannan.

Embodiment 99: The immunogenic nanoparticle of embodiment 98, wherein said targeting moiety comprises a mannan having a MW ranging from about 35 to about 60 kDa.

Embodiment 100: The immunogenic nanoparticle according to any one of embodiments 83-99, wherein said targeting moiety targets lymph nodes.

Embodiment 101: The immunogenic nanoparticle of embodiment 100, wherein said targeting moiety targets lymph node germinal centers.

Embodiment 102: The immunogenic nanoparticle of embodiment 101, wherein said surface of said nanoparticle is glycosylated.

Embodiment 103: The immunogenic nanoparticle of embodiment 102, wherein targeting moiety comprises a glycan-rich moiety.

Embodiment 104: The immunogenic nanoparticle of embodiment 103, wherein targeting moiety comprises the glycan-rich bacterial protein, lumazine synthase.

Embodiment 105: The immunogenic nanoparticle of embodiment 100, wherein said targeting moiety is selected from the group consisting of CpG, and a member of the amph-CpG family.

Embodiment 106: The immunogenic nanoparticle according to any one of embodiments 83-105, wherein said second binding moiety is adsorbed to said nanoparticle.

Embodiment 107: The immunogenic nanoparticle according to any one of embodiments 83-105, wherein said second binding moiety is covalently bound to said nanoparticle directly or through a linker.

Embodiment 108: The immunogenic nanoparticle according to any one of embodiments 1-107, wherein said nanoparticle contains a compound that facilitates cytosolic release from the endolysosomal compartment.

Embodiment 109: The immunogenic nanoparticle of embodiment 108, wherein said compound comprises an endo-osmolytic peptide.

Embodiment 110: The immunogenic nanoparticle of embodiment 108, wherein said compound comprises an endo-osmolytic peptide selected from the group consisting of MPG, Pep-1, and PPTG1) that destabilizes the in the endolysosomal membrane, antimicrobial peptide LL-37 with glutamic acid substituting for all basic residues, antimicrobial peptide melittin with glutamic acid substituting for all basic residues, and antimicrobial peptide bombolitin V with glutamic acid substituting for all basic residues.

Embodiment 111: The immunogenic nanoparticle according to any one of embodiments 1-110, wherein said nanoparticle contains a compound that increases immunogenicity.

Embodiment 112: The immunogenic nanoparticle of embodiment 111, wherein said compound is a compound that permits or induce the maturation of dendritic cells (DCs).

Embodiment 113: The immunogenic nanoparticle of embodiment 112, wherein said compound that permit or induce the maturation of dendritic cells (DCs) is selected from the group consisting of a lipopolysaccharide, TNF-alpha, and a CD40 ligand.

Embodiment 114: The immunogenic nanoparticle of embodiment 113, wherein said compound is selected from the group consisting of IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta, TNF-alpha, and hGH.

Embodiment 115: The immunogenic nanoparticle of embodiment 113, wherein said compound is selected from a cytokine shown in Table 16.

Embodiment 116: The immunogenic nanoparticle according to any one of embodiments 1-115, wherein said immunogenic nanoparticle is effective to raise an immune response when administered to a mammal.

Embodiment 117: The immunogenic nanoparticle of embodiment 116, wherein said nanoparticle is effective to raise a humoral immune response when administered to a mammal.

Embodiment 118: The immunogenic nanoparticle according to any one of embodiments 116-117, wherein said nanoparticle is effective to raise a cellular immune response when administered to a mammal.

Embodiment 119: The immunogenic nanoparticle according to any one of embodiments 116-118, wherein said nanoparticle is effective to raise one or more panels of neutralizing antibodies directed against the virus from which the viral protein(s) and/or protein fragments(s) are derived when administered to a mammal.

Embodiment 120: The immunogenic nanoparticle according to any one of embodiments 116-119, wherein said nanoparticle is effective to raise a protective immune response against the virus from which the antigen(s) are derived when administered to a mammal.

Embodiment 121: The immunogenic nanoparticle of embodiment 120, wherein said protective immune response is partially protective.

Embodiment 122: The immunogenic nanoparticle of embodiment 120, wherein said protective immune response is fully protective.

Embodiment 123: The immunogenic nanoparticle according to any one of embodiments 116-122, wherein said mammal is a human.

Embodiment 124: The immunogenic nanoparticle according to any one of embodiments 116-122, wherein said mammal is a non-human mammal.

Embodiment 125: The immunogenic nanoparticle according to any one of embodiments 1-124, wherein said nanoparticle is not hollow.

Embodiment 126: The immunogenic nanoparticle according to any one of embodiments 1-125, wherein said biocompatible polymer nanoparticle has a molecular weight greater than 10 kDa, or greater than about 20 kDa, or greater than about 30 kDa.

Embodiment 127: The immunogenic nanoparticle of embodiment 126, wherein said biocompatible polymer has a molecular weight less than about 100 kDa, or less than about 90 kDa, or less than about 80 kDa, or less than about 70 kDa, or less than about 60 kDa.

Embodiment 128: The immunogenic nanoparticle according to any one of embodiments 126-127, wherein said biocompatible polymer has a molecular weight ranging from about 30 kDa to about 60 kDa, or from about 35 kDa to about 55 kDa, or is about 40 kDa.

Embodiment 129: The immunogenic nanoparticle according to any one of embodiments 1-128, wherein said viral proteins or fragment(s) thereof do not comprise or do not consist of MERS-CoV RBD.

Embodiment 130: The immunogenic nanoparticle according to any one of embodiments 1-129, wherein said viral proteins or fragment(s) thereof do not comprise or do not consist of SARS-CoV-2 RBD.

Embodiment 131: The immunogenic nanoparticle according to any one of embodiments 1-130, wherein said adjuvant does not comprise the STING agonist cdGMP.

Embodiment 132: A pharmaceutical formulation comprising:
a population of immunogenic nanoparticles according to any one of embodiments 1-131; and
a pharmaceutically acceptable carrier.

Embodiment 133: The pharmaceutical formulation of embodiment 132, wherein said formulation is formulated for administration via a route selected from the group consisting of subcutaneous administration, intramuscular administration, inhalation, topical microneedle administration, and oral administration.

Embodiment 134: A method of inducing an immune response directed against a viral protein or viral protein fragment in a mammal, said method comprising:
administering to said mammal an effective amount of a population of immunogenic nanoparticles according to any one of embodiments 1-131, and/or a pharmaceutical formulation according to any one of embodiments 132-133.

Embodiment 135: The method of embodiment 134, wherein said viral proteins or fragments thereof are derived from a virus selected from the group consisting of corona viruses (including, but not limited to 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), SARS-CoV-2 (the novel coronavirus that causes coronavirus disease 2019, or COVID-19), and the like), human influenza viruses Types A and B, human Rhinovirus, respiratory syncytial virus (e.g., sin-SISH-uhl), parainfluenza viruses (HPIVs), measles virus, rubella virus, chickenpox/shingles virus, smallpox, chikungunya virus, hepatitis viruses, herpes viruses, human papilloma viruses (HPVs), polio virus, Norovirus, Rotavirus, Marburg virus, Ebola virus, Rabies, Smallpox, HIV, Hantavirus, Dengue, Molluscum contagiosum, Herpes simplex virus-1 (HSV-1), and Varicella-zoster virus (VZV).

Embodiment 136: The method of embodiment 134, wherein said viral proteins or fragments thereof are derived from a virus weaponized or studied as an agent for biological warfare.

Embodiment 137: The method of embodiment 136, wherein said virus is selected from the group consisting of the Bunyaviridae (especially Rift Valley fever virus), Ebolavirus, many of the Flaviviridae (especially Japanese encephalitis virus), Machupo virus, Marburg virus, Variola virus, yellow fever virus, viral encephalitis alphaviruses (e.g., venezuelan equine encephalitis, eastern equine encephalitis, western equine encephalitis), hantaviruses, Nipah virus, Tickborne encephalitis viruses, tickborne hemorrhagic fever viruses, and Yellow fever.

Embodiment 138: The method of embodiment 135, wherein said coronavirus comprises a member of the Betacoronavirus Genus.

Embodiment 139: The method of embodiment 138, wherein said coronavirus comprises a coronavirus that produces severe acute respiratory syndrome (SARS).

Embodiment 140: The method of embodiment 139, said coronavirus is a coronavirus selected from the group consisting of that SARS-CoV-2 (2019-nCoV), SARS-CoV-2, and MERS-CoV.

Embodiment 141: The method of embodiment 140, wherein said coronavirus is SARS-CoV-2 (2019-nCoV).

Embodiment 142: The method according to any one of embodiments 134-141, wherein said nanoparticle is effective to raise a humoral immune response when administered to a mammal.

Embodiment 143: The method according to any one of embodiments 134-142, wherein said nanoparticle is effective to raise a cellular immune response when administered to a mammal.

Embodiment 144: The method according to any one of embodiments 134-143, wherein said nanoparticle is effective to raise a neutralizing or protective antibody population directed against the virus from which the viral protein(s) and/or protein fragments(s) are derived when administered to a mammal.

Embodiment 145: The method according to any one of embodiments 134-144, wherein said nanoparticle is effect to raise a protective immune response against the virus from which the antigen(s) are derived when administered to a mammal.

Embodiment 146: A method for the prophylaxis and/or treatment of a viral infection in a mammal, said method comprising:

administering to said mammal an effective amount of a population of immunogenic nanoparticle according to any one of embodiments 1-124, and/or a pharmaceutical formulation according to any one of embodiments 132-133.

Embodiment 147: The method of embodiment 146, wherein said viral infection is produced by a virus selected from the group consisting of corona viruses (including, but not limited to 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), SARS-CoV-2 (the novel coronavirus that causes coronavirus disease 2019, or COVID-19), and the like), human influenza viruses Types A and B, human Rhinovirus, respiratory syncytial virus (e.g., sin-SISH-uhl), parainfluenza viruses (HPIVs), measles virus, rubella virus, chickenpox/shingles virus, smallpox, chikungunya virus, hepatitis viruses, herpes viruses, human papilloma viruses (HPVs), polio virus, Norovirus, Rotavirus, Marburg virus, Ebola virus, Rabies, Smallpox, HIV, Hantavirus, Dengue, Molluscum contagiosum, Herpes simplex virus-1 (HSV-1), and Varicella-zoster virus (VZV).

Embodiment 148: The method of embodiment 146, wherein said viral infection is produced by a virus weaponized or studied as an agent for biological warfare.

Embodiment 149: The method of embodiment 148, wherein said virus is selected from the group consisting of the Bunyaviridae (especially Rift Valley fever virus), Ebolavirus, many of the Flaviviridae (especially Japanese encephalitis virus), Machupo virus, Marburg virus, Variola virus, yellow fever virus, viral encephalitis alphaviruses (e.g., venezuelan equine encephalitis, eastern equine encephalitis, western equine encephalitis), hantaviruses, Nipah virus, Tickborne encephalitis viruses, tickborne hemorrhagic fever viruses, and Yellow fever.

Embodiment 150: The method of embodiment 147, wherein said coronavirus comprise a member of the Betacoronavirus Genus.

Embodiment 151: The method of embodiment 150, wherein said coronavirus comprises a coronavirus that produces severe acute respiratory syndrome (SARS).

Embodiment 152: The method of embodiment 151, said coronavirus is a coronavirus selected from the group consisting of that SARS-CoV-2 (2019-nCoV), SARS-CoV-2, and MERS-CoV.

Embodiment 153: The method of embodiment 152, wherein said coronavirus is SARS-CoV-2 (2019-nCoV).

Embodiment 154: The method according to any one of embodiments 146-153, wherein said method is effective to raise a humoral immune response in said mammal.

Embodiment 155: The method according to any one of embodiments 146-154, wherein said method is effective to raise a cellular immune response in said mammal.

Embodiment 156: The method according to any one of embodiments 146-155, wherein said method is effective to raise neutralizing antibody population directed against the virus.

Embodiment 157: The method according to any one of embodiments 146-156, wherein said method is effective to raise a protective immune response against the virus.

Embodiment 158: The method of embodiment 157, wherein said protective immune response is partially protective.

Embodiment 159: The method of embodiment 157, wherein said protective immune response is fully protective.

Embodiment 160: The method according to any one of embodiments 134-159, wherein said mammal is a human.

Embodiment 161: The method according to any one of embodiments 134-159, wherein said mammal is a non-human mammal.

It is noted that in any of the above-identified embodiments, where a protein or protein fragment or epitope is identified, immunogenic nanoparticles comprising a nucleic acid (e.g., a mRNA that when translated provides a protein or protein fragment/epitope) is also provided, e.g., as exemplified by the above embodiments insofar as they ultimately depend from any of Embodiments 3-12, and the like.

### Definitions

The terms “polypeptide”, “peptide”, and “protein” are used interchangeably herein to refer to a polymer of amino acid residues.

The term “viral protein” refers to a protein that is found in a virus. Illustrative viral proteins include but are not limited to the coronavirus spike protein (S-protein), and nucleocapsid protein (N-protein).

The terms “epitope” and “antigen determinant” as used herein may comprise viral protein fragments having a length ranging from about 6 to about 20 or even more amino acids, e.g., fragments as processed and presented by MHC class I molecules, preferably having a length of about 8 to about 10 amino acids, e.g., 8, 9, or 10, (or even 11, or 12 amino acids), or fragments as processed and presented by MHC class II molecules, preferably having a length of about 13 or more amino acids, e.g., 13, 14, 15, 16, 17, 18, 19, 20 or even more amino acids, wherein these fragments may be selected from any part of the amino acid sequence. These fragments are typically recognized by T cells in the form of a complex consisting of the peptide fragment and an MHC molecule.

A “B cell epitope” is the antigen portion binding to the immunoglobulin or antibody. These epitopes are typically fragments located on the outer surface of (native) protein or peptide antigens, having from about 5 to about 15 amino acids, or from about 5 to about 12 amino acids, or from about 6 to about 9 amino acids, which may be recognized by antibodies, e.g., in their native form. B-cell epitopes can be structural (e.g., one contiguous stretch of peptide sequence) or conformational (e.g., strung together by different pieces of the allergen that is not necessarily sequential but based on protein folding.

The term “T cell epitope” refers to a peptide derived from a protein that is recognized by the T-cell receptor (TCR) when bound to MHC molecules displayed on the cell surface of antigen-presenting cells (APCs).

The term “B-cell epitope” refers to solvent-exposed portions of an antigen (e.g., viral protein) that binds to secreted and cell-bound immunoglobulins.

An “immune response” refers to a specific reaction of the adaptive immune system to a particular antigen (so-called specific or adaptive immune response) or an unspecific reaction of the innate immune system (so-called unspecific or innate immune response). In certain embodiments the adaptive immune response refers to the core to specific reactions (adaptive immune responses) of the adaptive immune system. Particularly, it relates to adaptive immune responses to infections by viruses like e.g., CoV-2, and MERS coronaviruses. However, this specific response can be supported by an additional unspecific reaction (innate immune response).

The terms “cellular immunity” and “cellular immune response” refer typically to the activation of macrophages, natural killer cells (NK), antigen-specific cytotoxic T-lymphocytes, helper T cells, memory T cells and the release of various cytokines and chemokines in response to an antigen. In a more general way, cellular immunity is not restricted to the role of assisting antibody production but also the activation of cellular elements of the immune system that provide protective immune responses. The cellular immune response includes the role of activating antigen-specific cytotoxic T-lymphocytes that are capable of inducing cytotoxic cell death of cells in the body that display antigenic epitopes on their surface, such as virus-infected cells, cells with intracellular infectious agents, or cancer cells displaying tumor antigens; activating macrophages and natural killer cells, enabling them to destroy pathogens; and stimulating cells to secrete a variety of cytokines that influence the function of other cells involved in adaptive immune responses and innate immune responses. After an acute phase infection, the immune system also allows the development of memory B and T-cell responses, that confer long-term protective immunity, which are key to vaccine development.

The terms “humoral immunity” and “humoral immune response” refer typically to antibody production and the accessory processes that may accompany it. A humoral immune response may be typically characterized by a sequence of events that allow precursor B cells to develop into antibody producing cells, with or without the assistance of helper T cells that assist B-cell development, e.g., through cytokine production, germinal center formation, immunoglobulin isotype switching, immunoglobulin affinity maturation and the generation of memory B cells. Humoral immunity also typically may refer to the effector functions of antibodies, which include pathogen and toxin neutralization, classical complement activation, and opsonin promotion of phagocytosis and pathogen elimination.

An “adjuvant” in the broadest sense is typically a pharmacological or biological agent or composition that may strengthen or modify the intensity or quality of the immune response through recognition by elements of the innate immune system. This includes a category of pharmacological or biological components (often from infectious agents) that can induce immunological danger signals that are detected by the innate immune system by pattern-associated recognition receptors. Conventionally the adjuvant terminology, in the context of the invention, refers to a compound or composition that serves as an auxiliary substance for immunogens, often by way of being included or combined with the immunogens in an immunological carrier. . It is to be interpreted in a broad sense and refers to a broad spectrum of substances that are able to increase the immunogenicity of antigens incorporated into or co-administered with an adjuvant in question. In certain embodiments, this can include the immunogen itself (e.g., a nucleic acid endoing an immunogenic peptide), which, in addition to antigenic properties or display of epitopes to the cognate immune system, may also impart immunological danger signals to the innate immune system. In the context of the immunogenic nanoparticles described herein, an adjuvant will preferably enhance the specific immunogenic effect of the active agents (e.g., viral protein(s) and/or viral protein fragment(s)). While the term “adjuvant” is typically understood not to comprise agents that confer immunity by themselves, it has become clear that in certain embodiments, nucleic acids encoding antigens or epitopes may also be recognized by toll-like receptors that confer an adjuvant effect. An adjuvant typically functions by stimulating the innate immune system and antigen-presenting cells to enhance the antigen-specific immune response by providing immunological danger signals that has an impact on innate antigen presenting cells, e.g., promoting presentation of an antigen to the immune system or promoting inflammation, trafficking and maturation of the cellular elements of the innate immune response. In addition to promoting activation of the cognate or specific immune response, an adjuvant may also modulate the quality or differentiation of the antigen-specific immune response by e.g., shifting a dominant TH2-based antigen-specific response to a directed TH1-based antigen-specific response or vice versa. Accordingly, an adjuvant may favorably modulate cytokine expression/secretion, antigen presentation, type of immune response. This can also have an important bearing on the safety of the vaccine response, where TH2-mediated immunity has historically been shown to have negative connotations to coronavirus vaccine development.

A protein fragment refers to a peptide that is shorter than the full-length protein and comprises as least 5, or at least 8, or at least 10, or at least 12, or at least 16, or at least 20 continuous amino acids from the full-length protein. In certain embodiments a viral protein fragment comprises an epitope (e.g., a T cell epitope and/or a B cell epitope).

The term “STING agonist” refers to an agonist of the Stimulator of Interferon Genes receptor also known as transmembrane protein 173 (TMEM173)

The term “about” when used with respect to a numerical value refers to that value ±10%, or ±5%, ±3%, or ±2%, or ±1% of that value. In certain embodiments about refers to ±10% of the value. In certain embodiments about refers to ±5% of the value. In certain embodiments about refers to ±2% of the value.

As used herein, the term “targeting moiety” refers to any moiety that binds to a component of a cell. In some embodiments, the targeting moiety specifically binds to a component of a cell. Such a component can be referred to as a “target”, as a “marker” or as a “receptor”. In various embodiments a targeting moiety may be a polypeptide, glycoprotein, nucleic acid, small molecule, carbohydrate, lipid, aptamer etc. In some embodiments, a targeting moiety is an antibody or characteristic portion thereof. In some embodiments, a targeting moiety is a receptor or characteristic portion thereof. In some embodiments, a targeting moiety is a ligand or characteristic portion thereof. In some embodiments, a targeting moiety is a nucleic acid targeting moiety (e.g., an aptamer) that binds to a cell type specific marker. In some embodiments, a targeting moiety is a small molecule. In certain embodiments, the targeting moiety binds a receptor expressed on the surface of a cell. The targeting moiety, in some embodiments, binds a soluble receptor. In some embodiments, the soluble receptor is a complement protein or a pre-existing antibody. In certain embodiments, the targeting moiety is for delivery of the nanocarrier to antigen-presenting cells, T cells, or B cells. In some embodiments, the antigen-presenting cells are macrophages and dendritic cells. In other embodiments, the macrophages are located in the subcapsular sinus of lymph nodes. In still other embodiments, the antigen-presenting cells are dendritic cells. In some embodiments, the antigen-presenting cells are follicular dendritic cells or Langerhans cells. Specific non-limiting examples of targeting moieties include, but are not limited to, molecules that bind to CD11b, CD169, mannose receptor, DEC-205, CD11c, CD21/CD35, CX3CR1, Fc receptor or a toll-like receptor (TLR). In some embodiments, the molecule that binds any of the foregoing is an antibody or antigen-binding fragment thereof (e.g., an anti-CD169 antibody). In some embodiments, the molecule that binds a Fc receptor is one that comprises the Fc portion of an immunoglobulin (e.g., IgG). In other embodiments, the Fc portion of an immunoglobulin is a human Fc portion. In some embodiments, the molecule that binds CX3CR1 is CX3CL1 (fractalkine). Targeting moieties that bind CD169 include anti-CD169 antibodies and ligands of CD169, e.g., sialylated CD227, CD43, CD206, or portions of these ligands that retain binding function, e.g., soluble portions. In some embodiments, the molecule that may direct a nanoparticles vaccine to the lymph node germinal center may be a fragment of the complement system, which is activated at the surface of the nanoparticle nanocarrier by glycosylated particle components.

The terms “TH1-biased adjuvant”, TH1-preferential adjuvant”, and “TH1-promoting adjuvant” are used interchangeably and refer to an adjuvant that is defined in the literature (see, e.g., Liu et al. (2003) Nature Immunology, 4: 687-693) as an immunomodulator that is able to promote or trigger a TH1 immune response against a given antigen when used together with this antigen. A TH1 immune response is mediated by a T-helper type I CD4⁺ T-cell that promotes cell-mediated immunity, and is required for host defense against intracellular viral and bacterial pathogens. The generation of TH1 immunity by a vaccine adjuvant can be reflected by the production of TH1 cytokines, such as interferon-gamma, IL-2 and tumor necrosis factor-alpha or beta in the supernatant of splenocytes or peripheral blood lymphocytes that are collected from treated subject and cultured in the presence of an antigen. The TH1 differentiation pathway is controlled by a master transcription factor, T-bet. The control in such a demonstration would be to use a splenocyte population from TH1 a subject or the control group that has not been treated with the antigen and the adjuvant, or a subject or treatment group that was treated with the antigen only. In various embodiments, triggering or promoting a TH1 immune response is defined by the preferential induction of IFN-gamma over IL-4. e.g., as detected by harvesting peripheral blood mononuclear cells or splenocytes of a treated subject to perform an analysis, or assessing the isotype of the antibodies being produced against the vaccinating antigens, e.g., measurement of the IgG2a subclass that reflects immunoglobulin class switching under the influence of TH1 cells (as compared to IgG1a subclass switching under the influence of TH2 cells). The assessment of the induction of this cytokine can be carried out, by methods well known to those of skill in the art, for example, the use of ELISA to assess cytokine production in splenocyte supernatants, flow cytometry assessing IFN-gamma/IL-4 or IFN-gamma/IL-10 ratios in permeabilized mononuclear cells or determining IgG subclasses specific antibody titers by ELISA. In certain embodiments, the induction of IFN gamma and/or IgG2a and IgG2b upon stimulation of splenocytes with antigen and an adjuvant indicates that the adjuvant exert TH1-promoting immunostimulatory effects. In certain embodiments, alternatively or in combination with the first definition of triggering or promoting a TH1 immune response given above, triggering or promoting a TH1 immune response may further be defined by the absence (or the absence of an induction) of a TH2 immune response. A TH2 immune response is characterized by a detectable increase in IL-4, IL-5, IL-10, and IL-13 induction and/or the production of detectable IgG1 immunoglobulins when compared with non-treated splenocytes. The assessment of the induction of IL-4 and/or IL-10 can be carried out by methods well known to those of skill in the art, for example, the use of ELISA or flow cytometry on splenocytes. The assessment of the induction of an IgG1 is preferably carried out by ELISA or Western Blot as described in the example. In certain embodiments, alternatively or in combination with the two above definitions of triggering or promoting a TH1 immune response or promoting a TH immune response may further be defined by the generation of an increase in IFN gamma/IL-10 ratio and/or IFN gamma/IL-4 ratio and/or a decrease in IgG1/IgG2a ratio against a defined antigen. In certain embodiments change (increase or decrease as indicated above) in any of these ratio of more than 2 indicates that an adjuvant has TH1 properties. The assessment of the induction of each of the mentioned cytokines can be carried out by methods well known to those of skill in the art, for example, by ELISA or performance of RT-PCR on splenocytes.

A vaccine is typically understood to be a prophylactic or therapeutic material providing at least one antigen or antigenic function. The antigen or antigenic function may stimulate the body's cognate immune system to provide an adaptive immune response. It is often necessary that the antigenic portion is presented to the adaptive immune system by components of the innate immune system (antigen-presenting cells such as dendritic cells), and that the vaccine may include an adjuvant that promotes the ability of the innate immune system to perform the antigen-presenting function.

### BRIEF DESCRIPTION OF THE DRAWINGS

FIG. 1 illustrates development of immunogenic nanoparticles as described herein, e.g., for immunotherapy against COVID-2019.

FIG. 2, panels A-B, illustrates how biocompatible polymer (e.g., PLGA) nanoparticles (NPs) facilitate robust STING activation. Panel (A) illustrates one embodiment of a biocompatible polymer comprising a STING agonist, while panel (B) illustrates the mode of action of one such nanoparticle.

FIG. 3 illustrates the overall design toolbox in design a nano-enabled COVID-19 (or other viral targets) vaccination.

FIG. 4 illustrates the synthesis of PLGA nanoparticles for RBD delivery as a COVID-19 vaccine. PLGA Nanoparticles were fabricated using a double emulsion (w/o/w) method, followed by solvent removal. Briefly, PLGA and the Sting agonist, diABZi (MedChemExpress), were co-dissolved in dichloromethane, and RBD solution, provided by Dr. Francisco Ibarrondo at UCLA, was added to the PLGA solution to sonicate forming the primary emulsion (w/o). The primary emulsion was poured into aqueous solution containing surfactant and sonicated to form double emulsion (w/o/w). The double emulsion was stirred overnight to allow the evaporation of dichloromethane, and then washed by centrifugation to remove the non-encapsulated antigen and agonist

FIG. 5, panels A-C, shows RBD-specific antibody responses in C57BL/6 mice immunized with different vaccine formulations. Panel (A) Mice were intramuscularly vaccinated on day 0, 14, and 28 with 100 μL (50 μL/hind leg) of different vaccine formulations containing 5 μg of antigen. Since the antigen content of NPs was ˜35 μg per mg particle, the injected particle dose was 140 μg per mouse to achieve the required antigen dose (5 μg). The bare particles without antigen encapsulated working as controls were administered at the same particle dose of 140 μg per mouse. For control, the antigen alone was injected into the mice at the same antigen dose of 5 μg per mouse. Blood samples were collected from the venous plexus of the mice's posterior orbits. Sera was separated for later analysis. Panel (B) RBD-specific IgG and IgM titers in the serum on day 21 after first immunization. RBD-specific IgG and IgM in the serum were quantitatively determined by enzyme-linked immunosorbent assay (ELISA) in accordance with a protocol described previously (see, e.g., Liu et al. (2019) ACS Nano, 13 (4): 4778-4794). HRP-conjugated goat antibodies against mouse IgG (Sigma) and IgM (Abcam) were used as the diluted ratio of 1:20,000 for IgG and 1:10000 for IgM, respectively. Panel (C) RBD-specific IgG titers in the serum on day 35, 42, 49. Data were expressed as the mean±SEM (n=6). *p<0.05; **p<0.01; ***p<0.001.

### DETAILED DESCRIPTION

In various embodiments immunogenic nanoparticles capable of inducing an immune response directed against viral proteins and viruses containing the viral proteins as well as methods of use of the nanoparticles. In certain embodiments the nanoparticles are capable of inhibiting or preventing infection by a virus and accordingly various methods of immunization or vaccination using the nanoparticles that are provided.

In certain embodiments the immunogenic nanoparticles (e.g., nanoparticle-based vaccines) provide for the delivery of COVID-19 antigens (proteins and/or protein fragments (e.g., epitopes) and adjuvants (e.g., Stimulator of Interferon Genes (STING) agonists) to generate potent cellular and/or humoral immunity against the virus. In particular, in certain embodiments, the nanoparticles are designed to generate neutralizing antibodies that bind to the viral spike protein (S-protein) and/or viral spike protein epitopes (and/or to the N-protein and/or N-protein epitopes), with a view to provide a protective vaccination response against COVID-19. It will be recognized, however, that by using different viral proteins (or protein fragments) nanoparticles that induce an immune response directed against numerous other viruses are possible.

The basic platform is comprised of nanoparticles formed from a biocompatible polymer (e.g., poly (lactic-co-glycolic acid) (PLGA) copolymer), that has been approved for a host of therapeutic applications by the Food and Drug Administration (FDA) because of high biodegradability and biocompatibility. The biocompatible nanoparticles provide a versatile platform that can be custom-designed to incorporate proteins/peptides, nucleic acids, hydrophobic drugs, and combinations of these agents. The PLGA backbone can also be modified by the choice of the combination of copolymers as well as inclusion of additional polymeric or lipid components that can promote or alter the self-assembly of the particle components. Moreover, the particle surface can be functionalized with ligands or surface functionalities that target specific cell types.

To develop a COVID-19 vaccine, we initially selected the viral spike (S) protein, used by the SARS-CoV-2 virus for cellular entry. The inclusion of the S-protein is premised on the following data: (i) Preliminary studies suggesting that SARS-CoV-2 is quite similar to SARS-CoV, premised on the full-length genome phylogenetic analysis (see, e.g., Lu et al. (2020) Lancet, 6736: 1-10); (ii) SARS-CoV-2 virus uses the same SARS-CoV receptor (ACE-2) for entry into target cells (see, e.g., [Homann et al. (2020) bioRxiv, www.biorxiv.org/content/10.1101/2020.01.31.929042v1); (iii) Various reports related to SARS-CoV that suggest a protective role of both humoral and cell-mediated immune responses. For SARS-CoV, the generation of an antibody response to the surface S-protein has been shown to protect against infection in mouse models (see, e.g., Yang et al. (2004) Nature, 428: 561-564; Deming et al. (2006) PLoS Med. 3: e525; Graham et al. (2012) Nat. Med. 18: 1820-1826; and the like). Thus, the understanding of the similarity between SARS-CoV and COVID-19, also allows us to use the knowledge of the protective immunity against the former coronavirus for immunization against the COVID-19 strain.

It will be recognized, however, that using the teachings provided herein the incorporation of other viral proteins (and/or protein fragments) immunogenic nanoparticles directed against other viruses can readily be produced.

It will also be recognized that where an immunogenic nanoparticle comprising a protein or protein fragment is described herein, an immunogenic nanoparticle comprising a nucleic acid encoding the protein or protein fragment is also contemplated. It will be recognized that in certain embodiments, the protein or protein fragment comprises a single epitope, while in other embodiments the protein or protein fragment(s) comprise multiple epitopes that can be separate or can be provided as a fusion protein. Similarly, it will be recognized that in certain embodiments, nanoparticles comprising nucleic acid(s) encoding proteins comprising a single comprises a single epitope are provided, while in other embodiments nanoparticles comprising nucleic acid(s) encoding proteins multiple epitopes that can be separate or components of a fusion protein are provided.

Accordingly, in certain embodiments, an immunogenic nanoparticle is provided where the immunogenic nanoparticle comprises:
a nanoparticle formed from one or more biocompatible polymer(s);
one or more viral proteins or fragments thereof encapsulated within or attached to said biocompatible polymer where said the viral protein or fragment thereof comprises an antigen to which an immune response is to be induced by administration of said immunogenic nanoparticle to a mammal; and
an adjuvant.

In certain embodiments, an immunogenic nanoparticle is provided where the immunogenic nanoparticle comprises:
a nanoparticle comprising a biocompatible polymer; and
a nucleic encapsulated within or attached to said nanoparticle where said nucleic acid encodes one or more viral protein(s) or fragment(s) thereof that comprise one or more antigen(s) to which an immune response is to be induced by administration of said immunogenic nanoparticle to a mammal.

By way of illustration, a first-generation immunogenic (e.g., vaccinating) nanocarrier described herein incorporates the SARS-CoV-2 S-protein into a PLGA nanoparticle during the early aqueous synthesis phase. The S-protein is commercially available (e.g., from Sino Biological and can also be obtained through cellular transfection of an expression plasmid). In addition, identified cytotoxic or memory-inducing T-cell and B-cell epitopes in the S-protein are used to generate additional particles that can be used for providing vaccination nanocarriers. The use of the T-cell epitopes may offer advantages over the whole protein. For instance, these peptides are capable of binding to the MHC I and MHC II peptide groove by multiple strong forces such as hydrogen bonding and anchoring salt bridges, thereby promoting strong immune responses. Also, this strategy allows the incorporation of multiple epitopes to increase the valency and the efficacy of the vaccine. As illustrated in Example 1, in various embodiments the adjuvant in the nanoparticles comprises amidobenzimidazole (diABZI), a potent STING agonist. This embodiment, however, is illustrative and non-limiting.

Illustrative nanoparticles, nanoparticle materials, viral proteins and/or protein fragments, and adjuvants are described below. Additionally, various auxiliary agents that can be incorporated into the nanoparticle as well as targeting moieties that can be disposed on (e.g., attached to directly or through a linker) the immunogenic nanoparticle surface are described below.

### Proteins and Protein Fragments Comprising an Antigen/Epitope.

In certain embodiments the immunogenic nanoparticles described herein comprise one or more proteins or protein fragments that comprising one or more antigen(s)/epitopes to which an immune response is to be induced. In certain embodiments the proteins and/or protein fragments comprise a single antigen and/or epitope. In certain embodiments the nanoparticles comprise multiple antigens and/or epitopes. In certain embodiments the multiple antigens and/or epitopes are separate. In other embodiments the multiple antigens and/or epitopes are regions in a single polypeptide. In various embodiments the antigens and/or epitopes can be juxtaposed in a single peptide. In certain embodiments the antigens and/or epitopes can be separated from each other by single amino acids or by peptide linkers. In certain embodiments such peptide linkers comprise 2 amino acids, or 3 amino acids, or 4 amino acids, or 5 amino acids, or 6 amino acids, or 7 amino acids, or 8 amino acids, or 39 amino acids, or 10 amino acids, or 11 amino acids, or 12 amino acids, or 13 amino acids, or 14 amino acids, or 15 or more amino acids. In certain embodiments the amino acid or linker comprises a glycine (G), a serine (S), a lysine (K), and the like. In certain embodiments the amino acid linker comprises GG, or SS, or KK, or GGG, or SSS, or KKK, or combinations thereof.

The proteins and/or protein fragments for inclusion in the immunogenic nanoparticles described herein can be prepared by any of a number of means well known to those of skill in the art. For example, in certain embodiments, the proteins and/or protein fragments are isolated from wildtype organisms (e.g., wildtype Covid viruses). Such proteins and/or protein fragments (e.g., COVID spike protein) can be isolated, and/or fractionated, and/or purified using methods well known to those of skill in the art.

In certain embodiments the proteins and/or protein fragments can be recombinantly expressed and purified by methods well known to those of skill the art. It is noted that recombinant expression is well suited to the production of proteins that comprise multiple antigens and/or epitopes.

### Nucleic Acid(s) Encoding Proteins and Protein Fragments Comprising an Antigen/Epitope.

In certain embodiments the immunogenic nanoparticles described herein comprise one or more nucleic acids that encode proteins comprising one or more antigen(s)/epitopes to which an immune response is to be induced. In certain embodiments the nucleic acid comprises a DNA. In certain embodiments the nucleic acid comprises an mRNA.

The core principle behind mRNA as a technology for vaccination is to deliver the transcript of interest, encoding one or more immunogen(s), into the host cell cytoplasm where expression generates translated protein(s) to be within the membrane, secreted or intracellularly located. In certain embodiments the mRNA comprises one of two categories of mRNA constructs: 1) Non-replicating mRNA (NRM); and 2) Self-amplifying mRNA (SAM) constructs. NRM and SAM constructs typically comprise a cap structure, 5′ and 3′ untranslated regions (UTRs), an open-reading frame (ORF) endcoding the antigen(s) of interest, and a 3′ poly(A) tail (see, e.g., Pardi (2018) Nat. Rev. Drug Dis. 17: 261-279). SAMs differ from NRM constructs by additionally including genetic replication machinery derived from positive-stranded mRNA viruses, most commonly from alphaviruses such as Sindbis and Semliki-Forest viruses (see, e.g., Cheng et al. (2001) J. Virol. 75: 2368-2376; Ljungberg & Liljestrom (2015) Exp. Rev. Vacc. 14: 177-194; and the like). Generally, the ORF encoding viral structural proteins is replaced by the selected transcript of interest, and the viral RNA-dependent RNA polymerase is retained to direct cytoplasmic amplification of the replicon construct.

Once delivered to the cytosol, NRM constructs are immediately translated by ribosomes to produce the protein of interest (e.g., comprising the antigen(s)/epitope(s)), which can undergo subsequent post-translational modification. Similarly, SAM constructs can also be immediately translated by ribosomes to produce the replicase machinery necessary for self-amplification of the mRNA. Self-amplified mRNA constructs are translated by ribosomes to produce the protein of interest, which can undergo subsequent post-translational modification. The innate and adaptive immune responses detect the protein of interest and generate the desired immune responses.

In various embodiments, mRNA vaccine manufacturing begins with the generation of a plasmid DNA (pDNA) containing a DNA-dependent RNA polymerase promoter, such as T7 (see, e.g., Rong et al. (1998) Proc. Natl. Acad. Sci. USA, 95: 515-519) and the corresponding sequence for the mRNA construct. The pDNA is linearized to serve as a template for the DNA-dependent RNA polymerase to transcribe the mRNA, and subsequently degraded by a DNase process step. In various illustrative, but non-limiting embodiments the addition of the 5′ cap and the 3′ poly(A) tail can be achieved during the in vitro transcription step (see, e.g., Stepinski et al. (2001) RNA, 7: 1486-1495; Grudzien-Nogalska et al. (2007) Meth. Enzym. Ch. 431: 203-227; and the like) or enzymatically after transcription (see, e.g., Martin & Moss (1975) J. Bio Chem. 250: 9330-9335). Enzymatic addition of the cap can be accomplished by using, for example, guanylyl transferase and 2′-O-methyltransferase to yield a Cap 0 (^{N7Me}GpppN) or Cap 1 (^{N7Me}GpppN^{2′-OMe}) structure, respectively, while the poly-A tail can be provided through enzymatic addition via poly-A polymerase.

In various embodiments purification or the mRNA construct can be achieved with the application of high-pressure liquid chromatography (HPLC). The resultant drug substance is then formulated into drug nanoparticle delivery system described herein.

The nanoparticles are formulated so that the mRNA construct once released into the cytoplasm of a cell, effectively utilizes the translational machinery of the host cell to generate a sufficient quantity of the encoded immunogen that is presented appropriately to the immune system. In this regard, a number of parameters can readily be optimized for any particular nucleic acid construct. These include, but are not limited to amount/concentration of the nucleic acid in the delivery particle, 5′ capping efficiency and structure; UTR structure, length, and regulatory elements; modification of coding sequence; and poly-A-tail properties.

Thus, for example, the length of the 3′ UTR, 5′ UTR structures, and regulatory elements in both UTRs can impact transcription efficiency. Third, the 5′ 7-methylguanosine (m7G) cap of the mRNA molecule, linked via a triphosphate bridge to the first transcribed nucleotide, facilitates efficient translation, and blocks 5′-3′ exonuclease-mediated degradation. The specific cap structure can play an important role in both protein production and immunogenicity, with incomplete capping (5′ triphosphate) and Cap 0 structures shown to stimulate RIG-1 (see, e.g., Devarkar et al. (2016) Proc. Natl. Acad. Sci. USA, 113: 596-601; Xu et al. (2018) Protein Cell, 9: 246-253; Lassig & Hopfner, (2017) J. Biol. Chem. 292: 9000-9009; and the like). Additionally, 2-O′-unmethylated capped RNA can be sequestered by cellular IFN-induced proteins with tetratricopeptide repeats (IFIT1) that prevent the initiation of translation, or detected by the cytoplasmic RNA sensor MDA5. Typically, the choice of enzyme and reaction conditions are optimized in order to catalyze the highest percentage of cap formation. Additionally, the poly (A) tail and its properties such as length, can be optimized for translation and protection of the mRNA molecule (see, e.g., Goldstrohm & Wickens (2008) Nat. Rev. Mol. Cell Biol. 9: 337-344; Azoubel Lima et al. (2017) Nat. Struct. Mol. Biol. 24: 1057-1064; and the like)).

In certain embodiments codon optimization and modification of nucleotides can contribute to translation efficiency. For example, optimization of guanine and cytosine (GC) content can have a significant impact (see, e.g., Kudla et al. (2016) Plos Biol. 4: e180). The innate immune activation to mRNA can also influence its utility as a delivery system. The use of modified nucleosides, such as pseudouridine or N-1-methylpseudouridine to remove intracellular signaling triggers for protein kinase R (PKR) activation, can provide enhanced antigen expression and adaptive immune responses (see, e.g., Anderson et al. (2010) Nucleic Acids Res. 38: 5884-5892; Andries et al. (2015) J. Contr. Rel. 217: 337-344; Pardi et al. (2018) J. Exp. Med. 215: 1571-1588; and the like).

It will be appreciated that the foreign nucleic acid constructs and manufacturing methods are illustrative and non-limiting and using the teaching provided herein, numerous other constructs and manufacturing methods will be available to one of skill in the art.

### Nanoparticles Comprising One or More Biocompatible Polymers.

In various embodiments any of a number of biocompatible polymers can be used to form the immunogenic nanoparticles described herein. Such biocompatible polymers are well known to those of skill in the art and include but are not limited to polyesters (e.g., poly(lactic-co-glycolic acid), poly(glycolic acid), poly(lactic acid), poly(caprolactone), poly(butylene succinate), poly(trimethylene carbonate), poly(p-dioxanone), poly(butylene terephthalate), and the like), poly(ester amide)s (PEA) (see, e.g., Guerrero et al. (2015) J. Control Release,. 211: 105-117), polyurethanes and polyurethane copolymers (see, e.g., Cherng et al. (2013) Int. J. Pharmaceutics, 450(1-2): 145-162), polyanhydrides (e.g., poly[bis(p-carboxyphenoxy)methane], poly[bis(hydroxyethyl)terephthalate-ethyl orthophosphorylate/terephthaloyl chloride], see, e.g., Chang et al. (1983) Biomaterials, 4(2): 131-133), poly(ortho esters) (see, e.g., Nair et al. (2006) Adv. Biochem. Eng. Biotechnol. 102: 47-90; Park et al. (2005) Molecules, 10: 146-161), polyphosphoesters (e.g., polyphosphoesters Poly[bis(hydroxyethyl) terephthalate-ethyl orthophosphorylate/terephthaloyl chloride]), poly(alkyl cyanoacrylates) (e.g., poly(butyl cyanoacrylate), poly(p-hydroxyalkanoate)s, poly(hydroxybutyrate), poly(hydroxybutyrate-co-hydroxyvalerate, collagen, albumin, gluten, chitosan, hyaluronate, cellulose, alignate, and starch.

In certain embodiments the biocompatible polymer comprises one or more cationic polymers. It is noted that such cationic polymers are particular well suited for delivery of a nucleic acid (e.g., DNA or mRNA). Illustrative cationic polymers include, but are not limited to Poly-L-lysine (PLL), poly-ethylenimine (PEI; a branched cationic polymer, assisting in endosomal delivery), poly[(2-dimethylamino) ethyl methacrylate] (pDMAEMA), polyamidoamine (PAMAM) dendrimers, biodegradable poly(β-amino ester) (PBAE) polymers, poly(amino-co-ester) (PACE)-based polymers (branched amino acids, assisting in endosomal delivery), and the like (see, e.g., Wahane et al. (2020) Molecules, 25: 2866).

In certain embodiments the biocompatible polymer comprises one or more polymers selected from the group consisting of poly(lactic-co-glycolic acid) (PLGA), poly(glycolic acid) (PGA), poly(lactic acid) (PLA), poly(caprolactone) (PCL), poly(butylene succinate), poly(trimethylene carbonate), poly(p-dioxanone), poly(butylene terephthalate), poly(ester amide) (HYBRANE®), polyurethane, poly[(carboxyphenoxy) propane-sebacic acid], poly[bis(hydroxyethyl) terephthalate-ethyl orthophosphorylate/terephthaloyl chloride], poly(β-hydroxyalkanoate), poly(hydroxybutyrate), and poly(hydroxybutyrate-co-hydroxyvalerate). In certain embodiments the biocompatible polymer comprises poly(lactic-co-glycolic acid) (PLGA).

In one illustrative, but non-limiting embodiment the biocompatible polymer comprises PLGA comprising a lactide/glycolide molar ratio of about 50:50. In certain embodiments the biocompatible polymer (e.g., PLGA) incorporates polyethylene glycol (PEG), e.g., about 8% up to about 20% e.g., ˜2kDA or ˜5 kDA PEG.

In certain embodiments the nanoparticles (without attached ligand(s)) are of a size effective or preferred for phagocytic uptake by macrophages and/or dendritic cells. It has been reported in the literature that antigen-presenting cells (APCs) can phagocytose particles ranging in size from about 50 nm up to about 3 μm. Additionally, the fabrication of biocompatible polymer (e.g., PLGA) nanospheres ranging in size from nanometer to micrometer size has been described (see, e.g., Swider, et al. (2018) Acta Biomaterialia, 73: 38-51). Accordingly, in various embodiments, immunogenic nanoparticles ranging in size (e.g., average or median diameter) from about 50 nm up to about 3 μm are contemplated. In certain embodiments the nanoparticles range in size from about 50 nm, or from about 75 nm, or from about 100 nm, or from about 125 nm, or from about 150 nm, or from about 200 nm, or from about 250 nm, or from about 300 nm, or from about 350 nm, or from about 400 nm, or from about 450 nm, or from about 500 nm up to about 3 μm, or up to about 2.75 μm, or up to about 2.5 μm, or up to about 2.0 μm, or up to about 1.75 μm, or up to about 1.50 μm, or up to about 1.25 μm, or up to about 1 μm, or up to about 900 nm, or up to about 800 nm. In certain illustrative, but non-limiting embodiments, the nanoparticles have a mean or median size (e.g., diameter) that ranges in size from about 500 to about 800 nm.

In certain embodiments the nanoparticle may be entirely composed of a biocompatible polymer (e.g., PLGA), while in other embodiments, the nanoparticle may also comprise one or more lipids. In certain embodiments the lipid(s) include lipid components, capable of activating an immunological “danger signal” and/or binding to a nucleic acid. Illustrative lipids include, but are not limited to didodecyldimethylammonium bromide (DDAB), and 1,2-dioleoyloxy-3-trimethylammonium propane chloride (DOTAP). DOTAP is cationic lipid that can bind mRNA and nuclei acids, in addition to delivering dangers signals. Other suitable lipids include, but are not limited to cationic lipids (e.g., cationic lipids used in cationic lipid nanoparticles for Covid S protein mRNA delivery) which include, but are not limited to DOTMA (N-[1-(2,3-dioleyloxy)propyl]-N,N,N-trimethylammonium chloride), 1,2-dilinoleyloxy-3-dimethylaminopropane (DLinDMA), dilinoleylmethyl-4 dimethyl aminobutyrate (DLin-MC3-DMA), C12-200 (Lipid 5), 3-(dimethylamino) propyl(12Z,15Z)-3-[(9Z,12Z)-octadeca-9,12-dien-1-yl]henicosa-12,15-dienoate (DMAP-BLP), (2Z)-non-2-en-1-yl 10-[(Z)-(1-methylpiperidin-4 yl)carbonyloxy]nonadecanoate (L101), and the like. In certain embodiments, inclusion of lipid can also be used to fine-tune the particle size and/or charge.

In certain embodiments the immunogenic nanoparticle according to any one of claims 22-24, wherein said lipid comprises up to 25%, [molar percentage (excluding viral proteins or fragment(s) thereof)] of the nanoparticle. In certain embodiments the lipid comprises about 1%, or about 2%, or about 3%, or about 4%, or about 5%, or about 6%, or about 7% up to about 25%, or up to about 20%, or up to about 15%, or up to about 10% (molar percentage (excluding viral proteins or fragment(s) thereof)) of the nanoparticle.

Methods of making polymeric nanoparticles are well known to those of skill in the art (see, e.g., Marin et al. (2013) Int. J. Nanomed., 8: 3071-3091, and references therein).

In certain embodiments the antigen(s) are encapsulated within the nanoparticle and this can readily be accomplished by combining the antigen(s) with the biocompatible polymer during nanoparticle synthesis. In certain embodiments the antigen(s) are covalently attached to the surface of the nanoparticle, e.g., by adsorption or by coupling directly or using a linker.

Viral Proteins or Viral Protein Fragments Antigen(s) for Incorporation into Immunogenic Nanoparticles.

In various embodiments the viral proteins (antigen(s)) comprising the immunogenic nanoparticles described herein comprise one or more antigenic protein/peptide sequence(s) to which it is desired to raise an immune response. It will be recognized that in certain embodiments the immunogenic nanoparticles comprise a single viral protein or protein fragment (e.g., a single epitope), while in other embodiments the immunogenic nanoparticles comprise 2, 3, 4, 5, 6, 7, 8, 9, or 10, or more viral protein(s) or protein fragment(s) (e.g., epitope(s). In certain embodiments the nanoparticles may be comprised comprise of two or more viral proteins or protein fragments (e.g., epitopes). In certain embodiments viral protein fragments can be provided as separate molecules/moieties or as peptide fragments that are linked together by appropriate spacers. These are also known as string on-bead vaccines or multi-epitope vaccines in certain embodiments the viral protein fragments (e.g., antigenic epitopes) can be provided so that they are presented as separate epitopes on a single molecule/moiety (e.g., as separate epitopes on a single protein). In certain embodiments, the viral protein(s) or protein fragment(s) (s) are disposed within the biocompatible polymer, while in other embodiments one or more of the viral protein(s) or protein fragment(s) can be provided attached to the surface of the biocompatible polymer.

In certain embodiments the above-described single antigen, or multiple antigen protein(s) can be provided using an immunogenic nanoparticle comprising a nucleic acid (e.g., mRNA) that encodes the single- or multiple-antigen protein(s).

In this regard, it is noted that such approaches facilitate the production of vaccines that improve T-cell B-cell cooperation. Such a “string-of bead-vaccines” approach as has previously been used for cytomegalovirus and influenza (see, e.g., Schubert & Kohlbacher (2016) Genome Med. 8: 9; Whitton et al. (1993) J. Virol. 67:348-352; Fomsgaard et al. (1999) Vaccine, 18: 681-691; and the like). In certain embodiments this entails the selection and combination of epitopes from diverse SARS-CoV2 antigens (M, N, E and S proteins) that are spliced together with the assistance of spacer sequences (e.g., cleavable spacer sequences). Similar outcomes can also be achieved by the design of DNA or RNA minigenes, as demonstrated by for influenza (see, e.g., Fomsgaard et al. (1999) Vaccine, 18: 681-691). Without being bound to a particular theory, it is believed to be possible to use suitably designed nanocarriers as described herein to deliver multi-epitope peptide sequences or minigene nucleic acid constructs to the host immune system for antigen presentation.

Typically the immunogenic nanoparticles described herein are provided to induce an immunogenic response directed against a viral antigen/epitope and thereby against a virus (viral strain) that harbors the antigen/epitope. Thus, in various embodiments the immunogenic nanoparticles described herein comprise a viral protein (e.g., a coronavirus spike (S) protein, or nucleocapsid (N) protein) or fragments thereof, e.g., viral epitopes. In certain embodiments the immunogenic nanoparticle comprises a T cell epitope (e.g., to induce cell-mediated immunity), a B cell epitope (e.g., to induce humoral immunity), or both a T cell epitope and a B cell epitope (to induce both cell-mediated and humoral immunity).

It is noted that in various embodiments, nanoparticles comprising multiple epitopes are provided (multi-epitope vaccines). In this regard it is noted that although initial efforts focused on achieving protection by generating neutralizing antibodies, a challenge for COVID-19 vaccine developers is uncertainty about the immunological correlates of vaccine efficiency. While contemporary vaccine trials suggest that IgG levels provide a good proxy for vaccine efficacy, it is known that neutralizing antibodies do not provide viral clearance from infected sites. This typically requires the participation of cytotoxic CD8+ T-cells as well as the cooperation of CD4+ T-cells. Accordingly, with respect to vaccine design, consideration is given as to the extent reliance is put on the sterilizing effects of antibodies as compared to the additional contribution of T-cells, including providing long term efficacy. Not only do activated CD4+ and cytotoxic CD8+ T-cells play a critical role in defense against acute viral infections, but evidence has been provided that during mild or asymptomatic infections, it is possible to observe the appearance of T-cells without significant antibody production (see, e.g., Sekine et al. 92929) Cell, 183: 158-168; Le Bert et al. (2020) Nature, 584: 457-462). This includes the appearance of T-cells in the circulation of sero-negative family members exposed to COVID-19. In addition, epitope mapping studies have shown the appearance of cross-reactive T-cell responses to the spike or membrane (M) proteins in a total of 28% of healthy blood donors before the onset of the pandemic (Sekine et al. 92929) Cell, 183: 158-168). This highlights the possibility that non-spike proteins can play an important role in cross-reactive immunity to multiple coronaviruses, a finding that is further corroborated by the detection of cross-reactive T-cells in 20-50% of uninfected people in high impact COVID-19 communities (see, e.g., Doshi (2020) BMJ, 370:m3563).

Accordingly, in various embodiment the immunogenic nanoparticle provided herein present individual epitopes or epitope combinations. In certain embodiments, the primary goal of these particles is to enhance T-cell responses and to obtain improved cooperation between T- and B-cells for boosting the production of neutralizing antibodies. In certain embodiments the immunogenic nanoparticles described herein comprise multiple epitopes and act as multi-eptitope vaccines.

An example of a multi-peptide (multi-epitope) is the United Biomedical.112 UB-612 vaccine which is comprised of 8 components and was designed to induce a combination of neutralizing antibodies plus T-cell responsivity through the inclusion of an S1-RBD-sFc fusion protein, 6 synthetic peptides (one universal plus 5 SARS-CoV-2-derived peptides), a proprietary CpG oligonucleotide (TLR-9 binding agonist) and an aluminum phosphate adjuvant. Vaccination studies in guinea pigs and rats demonstrated that the generation of high titers of neutralizing antibodies against S1-RBD, robust cellular immunity and TH1 skewing of the immune response. Subsequent challenge studies in a nonhuman primate animal model confirmed disease prevention and reduction of viral load.

Another example of multi-epitope vaccine design, aiming for durable immunity through a combination of B- and T-cell epitopes was recently demonstrated by Yarmarkovich et al. (2020) Cell Reports Med., 1:100036. These investigators developed 65×33mer-peptides. Epitope selections included evolutionarily conserved coronavirus sequences, including T-cell epitopes for population-based HLA coverage, in addition to the inclusion of linear and conformational B-cell epitopes. To maximize immunogenicity, only viral regions with the highest degree of dissimilarity to the human immunopeptidome were chosen. This vaccine design also allows conjugated TH1 adjuvants to be included with the peptides. While the efficacy of this design still awaits animal experimentation, we have outlined the success that was achieved by COVAXX using their 8-component multi-epitope vaccine (UB-612) to induce protective immune responses in rats, guinea pigs and in a nonhuman primate model (Guirakhoo, et al. (2021) bioRxiv Preprint 2021, //doi.org/10.1101/2020.11.30.399154.).

Multi-epitope vaccine design can also allow the generation of cross-reactive immunity that deliberately expands the contribution of different types of memory T-cells (Lipsitch et al. (2020) Nat. Rev. Immunol., 20: 709-713.). The feasibility of this approach is supported by a number of studies showing broad-based T-cell reactivity in 20%-50% of people with no known exposure to SARS-CoV-2 (Doschi supra.).

In certain embodiments the single-epitope or multiple-epitope immunogenic nanoparticles described herein comprises viral proteins that include, but are not limited to antigenic proteins, or epitopes derived from a virus selected from the group consisting of corona viruses (including, but not limited to 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), SARS-CoV-2 (the novel coronavirus that causes coronavirus disease 2019, or COVID-19), and the like), human influenza viruses Types A and B, human Rhinovirus, respiratory syncytial virus (e.g., sin-SISH-uhl), parainfluenza viruses (HPIVs), measles virus, rubella virus, chickenpox/shingles virus, smallpox, chikungunya virus, hepatitis viruses, herpes viruses, human papilloma viruses (HPVs), polio virus, Norovirus, Rotavirus, Marburg virus, Ebola virus, Rabies, Smallpox; HIV, Hantavirus; Dengue, Molluscum contagiosum, Herpes simplex virus-1 (HSV-1), Varicella-zoster virus (VZV), and the like. In addition, many viral agents have been weaponized or studied as agents for biological warfare, including some of the Bunyaviridae (especially Rift Valley fever virus), Ebolavirus, many of the Flaviviridae (especially Japanese encephalitis virus), Machupo virus, Marburg virus, Variola virus, yellow fever virus, viral encephalitis alphaviruses (e.g., venezuelan equine encephalitis, eastern equine encephalitis, western equine encephalitis), hantaviruses, Nipah virus, Tickborne encephalitis viruses, tickborne hemorrhagic fever viruses, and Yellow fever. As explained below antigenic proteins as well as specific T cell and B cell epitopes are known for these, and other viruses.

Coronavirus Antigens/Epitopes.

Coronaviruses that produce severe acute respiratory syndrome (SARS) pose an extremely significant threat to human health. In particular, SARS-CoV-2 is the causative agent in a worldwide pandemic. In certain embodiments the viral protein(s) used in the immunogenic nanoparticles or the viral proteins encoded by the nucleic acids used in the nanoparticles described herein comprise a full-length coronavirus spike protein (S-protein) or a fragment thereof (e.g., an S1 or S2 subunit, or an epitope from the full-length spike protein), and/or a full-length nucleocapsid protein (N-protein) or a fragment thereof (e.g., an epitope from the full-length nucleocapsid protein), and/or a full-length membrane protein (M-protein) or fragment thereof (e.g., an epitope of the full-length membrane protein).

The spike protein (S-protein) mediates receptor binding and membrane fusion. Spike protein contains two subunits, S1 and S2. S1 contains a receptor binding domain (RBD), that is responsible for recognizing and binding with the cell surface receptor. S2 subunit is the “stem” of the structure, which contains other basic elements needed for membrane fusion. The spike protein is the common target for neutralizing antibodies and vaccines. It has been reported that SARS-CoV-2 (2019-nCoV) can infect the human respiratory epithelial cells through interaction with the human ACE2 receptor. Indeed, the recombinant Spike protein can bind with recombinant ACE2 protein.

In certain embodiments the viral protein(s) used in the immunogenic nanoparticles or the viral proteins encoded by the nucleic acids used in the nanoparticles described herein comprise a full-length spike (S) protein or fragment thereof that comprises variant Covid spike proteins. In this regard, it is noted that three variants are of particular concern. These are termed the UK, South African and Brazil variants (B.1.1.7, also known as VOC 202012/01 with E484K mutation) or 20I/501Y.V1; B.1.351 (also known as 20H/501Y.V2); and P.1 (also known as 501Y.V3). Additionally, another variant has been circulating at a dominant level in California, USA, termed 1.427/429 (also 111 known as CAL.20C). Accordingly in certain embodiments, the viral protein(s) used in the immunogenic nanoparticles or the viral proteins encoded by the nucleic acids used in the nanoparticles described herein comprise features of one or more of these (or other) variants. In certain embodiments the viral protein(s) used in the immunogenic nanoparticles or the viral proteins encoded by the nucleic acids used in the nanoparticles described herein comprise one or more mutations selected from the group consisting of D614G, E484K, L452R, E484Q, and E484K.

The nucleocapsid protein (N-protein) is the most abundant protein in coronavirus. The N-protein is a highly immunogenic phosphoprotein, and it is normally very conserved. The N-protein of coronavirus is often used as a marker in diagnostic assays.

In certain embodiments the viral protein(s) used in the immunogenic nanoparticles comprise an epitope of the spike protein and/or an epitope of the nucleocapsid protein. In certain embodiments the viral protein(s) used in the immunogenic nanoparticles comprise a T cell epitope and/or a B cell epitope of the spike protein and/or a T cell epitope and/or a B cell epitope of the nucleocapsid protein.

The amino acid sequences of coronavirus spike proteins and nucleocapsid proteins as well as T cell epitopes and B cell epitopes thereof are well known to those of skill in the art.

SARS-CoV, SARS-CoV-2, and MERS-CoV Epitopes.

SARS-CoV causes Severe Acute Respiratory Syndrome (SARS). The newly identified coronavirus, SARS-CoV-2 (2019-nCoV) belongs to the Betacoronavirus genus, which also includes SARS CoV (2003) and MERS CoV (2012). According to the World Health Organization (WHO), the first human cases appeared in southern China in November 2002. SARS-CoV may have originated in bats and were transmitted to other animals before infecting humans. During the 2002-2003 epidemic, more than 8,000 people in 26 countries around the world contracted SARS. The outbreak was contained in mid-2003 with the implementation of infection control practices such as isolation and quarantine. Since then, a handful of cases have occurred due to laboratory accidents.

Similarly, SARS-CoV-2 causes COVID-19 which can also result in Severe Acute Respiratory Syndrome (SARS). This new coronavirus appeared in Wuhan, China, in late December 2019 after health officials noticed an increase in pneumonia cases with no known cause. Though the virus likely evolved from an animal source, its exact source is unknown. Within a few months, SARS-CoV-2 has spread to hundreds of countries around the world after being transmitted through person-to-person contact resulting in the most significant pandemic in recent times.

MERS-CoV causes Middle East Respiratory Syndrome (MERS). According to WHO, it emerged in September 2012 in Saudi Arabia, although initial cases were later traced back to Jordan. Humans contract MERS-CoV through contact with camels that have contracted the infection. The virus is also transmitted by coming into very close contact with a person who has the infection. Since 2012, 27 countries have reported more than 2,400 MERS cases. To date, the majority of cases have occurred in Saudi Arabia. In 2015, an outbreak in South Korea led to 186 cases and 36 deaths. According to the CDC, this outbreak originated with a traveler returning from the Middle East. According to the European Center for Disease Prevention and Control (ECDPC), there were more than 200 cases of MERS-CoV reported in 2019.

In view of this it will be recognized that immunogenic nanoparticles that can induce an immune response directed to SARS-causing coronaviruses are an important addition to the healthcare arsenal.

In various illustrative, but non-limiting embodiments, the viral protein(s) and/or protein fragment(s) (e.g., viral protein epitopes) are incorporated into the nanoparticle during the early aqueous synthesis phase. Numerous viral proteins (e.g., S-protein, N-protein) are commercially available (e.g., from Sino Biological) or can be produced in the laboratory by publicly available expression plasmids that can be transfected into mammalian tissue culture cells or a baculovirus system. Alternatively, such proteins can readily be expressed using recombinant expression systems by methods well known to those of skill in the art. Similarly, many viral protein fragments (e.g., epitopes) are commercially available, or can be synthesized to order by commercial suppliers.

As noted above it is also possible to use identified cytotoxic T-cell and B-cell epitopes on the S-protein (or N and M-proteins) to generate immunogenic nanoparticles. The use of the T-cell epitopes may offer advantages over the whole protein. For instance, these peptides are capable of binding to the MHC I or II peptide groove by multiple strong forces such as hydrogen bonding and anchoring salt bridges, thereby promoting strong immune responses. Also, this strategy allows the use of multiple epitopes to increase the valency and the efficacy of the vaccine. In addition to the availability of already-identified epitopes in the literature (see, e.g., Baruah et al. (2020) J. Med. Virol. 92:495-500; Ahmed et al. (2020) Viruses, 12: 254; Bhattacharya et al. (2020) J. Med. Virol. doi.org/10.1002/jmv.25736, and the like), we have also been able to use the publicly available NIAID-supported Immune Epitope Database (IEDB; www.iedb.org) to demonstrate that we can identify the presence of T-cell epitopes displayed on S-protein in the murine database. Table 1 illustrates five epitope sequences identified in the IEDB database to facilitate the performance of proof-of-principal animal studies. The same source can also be used to find additional human epitopes on other viral antigens, including the identification of B-cell epitopes. The identification of immunogenic peptide sequence by the IEDB has been proven to be immunologically relevant in animal and human research.

By way of illustration, we used the NIAID-supported IEDB (//www.iedb.org) to make predictions about possible 15-mer peptides for Spike protein. Five top-scoring peptides were selected by setting cut-off values of IC50 for the predicted binders at 500 nM. We selected the T-cell epitopes that do not overlap with the human proteome through sequence lining. These epitopes are illustrated in Table 1, and in certain embodiments, the immunogenic nanoparticles described herein comprise one or more of these epitopes. In certain embodiments the immunogenic nanoparticles comprise one of the epitopes shown in Table 1. In certain embodiments the immunogenic nanoparticles comprise two of the epitopes shown in Table 1. In certain embodiments the immunogenic nanoparticles comprise three of the epitopes shown in Table 1. In certain embodiments the immunogenic nanoparticles comprise four of the epitopes shown in Table 1. In certain embodiments the immunogenic nanoparticles comprise all of the epitopes shown in Table 1.

It is noted that when it is stated below that an immunogenic nanoparticle comprises an “epitope” it will be recognized that the immunogenic particle comprises a protein or protein fragment and/or one or more nucleic acid(s) (e.g., mRNA(s)) that encode the recited epitope(s).

TABLE 1
T cell epitopes of SARS-CoV-2 spike (S) protein

| | | | | SEQ |
|---|---|---|---|---|
| | T cell | Specifica- | | ID |
| Protein | epitope | tions | Sequence | NO |
| Spike (S) | S1 | 1069-1083 | PAQEKNFTTAPAICH | 1 |
| protein of | S2 | 1046-1060 | GYHLMSFPQSAPHGV | 2 |
| SARS- | S3 | 684-698 | ARSVASQSIIAYTMS | 3 |
| CoV-2 | S4 | 197-211 | IDGYFKIYSKHTPIN | 4 |
| | S5 | 901-915 | QMAYRFNGIGVTQNV | 5 |

Other SARS-CoV derived T cell epitopes that appear identical in SARS-CoV-2 are also well known to those of skill in the art. Thus, in certain embodiments the immunogenic nanoparticle described herein in addition to one or more of the epitopes listed in Table 1, or as an alternative to the epitopes listed in Table 1 can comprise one or more of the T cell epitopes shown in Table 2. In certain embodiments the immunogenic nanoparticles comprise an S-protein epitope shown in Table 2. In certain embodiments the immunogenic nanoparticles comprise an N-protein epitope shown in Table 2. In certain embodiments the immunogenic nanoparticles comprise both an S-protein epitope and an N-protein epitope shown in Table 2. In certain embodiments the immunogenic nanoparticles comprise at least two epitopes shown in Table 2. In certain embodiments the immunogenic nanoparticles comprise at least three epitopes shown in Table 2. In certain embodiments the immunogenic nanoparticles comprise at least four epitopes shown in Table 2. In certain embodiments the immunogenic nanoparticles comprise at least five epitopes shown in Table 2.

TABLE 2
SARS-CoV-derived T cell epitopes obtained using
positive T cell assays that are identical in SARS-
CoV-2 (From Ahmed et al. (2020) Viruses, 12: 254).

| | | | | MHC | SEQ |
|---|---|---|---|---|---|
| Pro- | IEDB | | | Allele | ID |
| tein | ID | Epitope | MHC Allele | Class | NO |
| N | 125100 | ILLNKHID | HLA-A02: 01 | 1 | 6 |
| N | 1295 | AFFGMSRIGMEVTPSGTW | NA | NA | 7 |
| N | 190494 | MEVTPSGTWL | HLA- | I | 8 |
| | | | B*40: 01 | | |
| N | 21347 | GMSRIGMEV | HLA-A02: 01 | I | 9 |
| N | 27182 | ILLNKHIDA | HLA-A02: 01 | I | 10 |
| N | 2802 | ALNTPKDHI | HLA- | I | 11 |
| | | | A*02: 01 | | |
| N | 28371 | IRQGTDYKHWPQIAQFA | NA | NA | 12 |
| N | 31166 | KHWPQIAQFAPSASAFF | NA | NA | 13 |
| N | 34851 | LALLLLDRL | HLA-A02: 01 | I | 14 |
| N | 37473 | LLLDRLNQL | HLA-A02: 01 | I | 15 |
| N | 37611 | LLNKHIDAYKTFPPTEPK | NA | NA | 16 |
| N | 38881 | LQLPQGTTL | HLA-A02: 01 | I | 17 |
| N | 3957 | AQFAPSASAFFGMSR | NA | II | 18 |
| N | 3958 | AQFAPSASAFFGMSRIGM | NA | NA | 19 |
| N | 55683 | RRPQGLPNNTASWFT | NA | I | 20 |
| N | 74517 | YKTFPPTEPKKDKKKK | NA | NA | 21 |
| S | 100048 | GAALQIPFAMQMAYRF | HLA- | II | 22 |
| | | | DRA*01: 01 | | |
| | | | HLA- | | |
| | | | DRB1*07: 01 | | |
| S | 100300 | MAYRFNGIGVTQNVLY | HLA- | II | 23 |
| | | | DRB104: 01 | | |
| S | 100428 | QLIRAAEIRASANLAATK | HLA- | II | 24 |
| | | | DRB104: 01 | | |
| S | 16156 | FIAGLIAIV | HLA-A02: 01 | I | 25 |
| S | 2801 | ALNTLVKQL | HLA- | I | 26 |
| | | | A*02: 01 | | |
| S | 36724 | LITGRLQSL | HLA-A2 | I | 27 |
| S | 44814 | NLNESLIDL | HLA-A02: 01 | I | 28 |
| S | 50311 | QALNTLVKQLSSNFGAI | HLA- | II | 29 |
| | | | DRB104: 01 | | |
| S | 54680 | RLNEVAKNL | HLA- | I | 30 |
| | | | A*02: 01 | | |
| S | 69657 | VLNDILSRL | HLA-A02: 01 | I | 31 |
| S | 71663 | VVFLHVTYV | HLA- | I | 32 |
| | | | A*02: 01 | | |

Ahmed et al. (supra.) estimated population coverages for various combinations of MHC alleles associated with 102 epitopes (see, Table 3). They determined sets of epitopes associated with MHC alleles with maximum population coverage, to facilitate the development of vaccines against SARS-CoV-2. In certain embodiments the immunogenic nanoparticles described herein comprise one or more of the epitopes listed in Table 3. In certain embodiments the immunogenic nanoparticles described herein comprise at least two of the epitopes listed in Table 3. In certain embodiments the immunogenic nanoparticles described herein comprise at least three of the epitopes listed in Table 3. In certain embodiments the immunogenic nanoparticles described herein comprise at least four of the epitopes listed in Table 3. In certain embodiments the immunogenic nanoparticles described herein comprise at least five of the epitopes listed in Table 3.

Shows a set of the SARS-CoV-derived spike (5) and nucleocapsid (N) protein T cell epitopes (obtained from positive MHC binding assays) that are identical in SARS-CoV-2 and that maximize estimated population coverage globally ((From Ahmed et al. (2020) Viruses, 12: 254). Epitopes that were also tested for positive T cell response (listed also in Table 2) are shown in bold. Epitopes that lie within the SARS-CoV receptor-binding motif are underlined.

TABLE 3

| | | | Global | |
|---|---|---|---|---|
| | MHC | | Accumulated | SEQ |
| | Allele | | Population | ID |
| Epitope | Class | MHC Allele | Coverage | NO |
| FIAGLIAIV | I | HLA-A*02: 01 | 39.08 | 33 |
| GLIAIVMVTI | | | | 34 |
| IITTDNTFV | | | | 35 |
| ALNTLVKQL | | | | 36 |
| LITGRLQSL | | | | 37 |
| LLLQYGSFC | | | | 38 |
| LQYGSFCT | | | | 39 |
| NLNESLIDL | | | | 40 |
| RLDKVEAEV | | | | 41 |
| RLNEVAKNL | | | | 42 |
| RLQSLQTYV | | | | 43 |
| VLNDILSRL | | | | 44 |
| VVFLHVTYV | | | | 45 |
| ILLNKHID | | | | 46 |
| FPRGQGVPI | | | | 47 |
| LLLLDRLNQ | | | | 48 |
| GMSRIGMEV | | | | 49 |
| ILLNKHIDA | | | | 50 |
| ALNTPKDHI | | | | 51 |
| LALLLLDRL | | | | 52 |
| LLLDRLNQL | | | | 53 |
| LLLLDRLNQL | | | | 54 |
| LQLPQGTTL | | | | 55 |
| AQFAPSASA | | | | 56 |
| TTLPKGFYA | | | | 57 |
| VLQLPQGTTL | | | | 58 |
| GYQPYRVVVL | I | HLA-A*24: 02 | 55.48 | 59 |
| PYRVVVLSF | | | | 60 |
| LSPRWYFYY | | | | 61 |
| DSFKEELDKY | I | HLA-A*01: 01 | 66.78 | 62 |
| LIDLQELGKY | | | | 63 |
| PYRVVVLSF | | | | 64 |
| GTTLPKGFY | | | | 65 |
| VTPSGTWLTY | | | | 66 |
| GSFCTQLNR | I | HLA-A*03: 01 | 76.14 | 67 |
| GVVFLHVTY | | | | 68 |
| AQALNTLVK | | | | 69 |
| MTSCCSCLK | | | | 70 |
| ASANLAATK | | | | 71 |
| SLIDLQELGK | | | | 72 |
| SVLNDILSR | | | | 73 |
| TQNVLYENQK | | | | 74 |
| CMTSCCSCLK | | | | 75 |
| VQIDRLITGR | | | | 76 |
| KTFPPTEPK | | | | 77 |
| KTFPPTEPKK | | | | 78 |
| LSPRWYFYY | | | | 79 |
| ASAFFGMSR | | | | 80 |
| ATEGALNTPK | | | | 81 |
| QLPQGTTLPK | | | | 82 |
| QQQGQTVTK | | | | 83 |
| QQQQGQTVTK | | | | 84 |
| SASAFFGMSR | | | | 85 |
| SQASSRSSSR | | | | 86 |
| TPSGTWLTY | | | | 87 |
| GSFCTQLNR | I | HLA-A*11: 01 | 83.39 | 88 |
| GVVFLHVTY | | | | 89 |
| AQALNTLVK | | | | 90 |
| MTSCCSCLK | | | | 91 |
| ASANLAATK | | | | 92 |
| SLIDLQELGK | | | | 93 |
| SVLNDILSR | | | | 94 |
| TQNVLYENQK | | | | 95 |
| CMTSCCSCLK | | | | 96 |
| VQIDRLITGR | | | | 97 |
| KTFPPTEPK | | | | 98 |
| KTFPPTEPKK | | | | 99 |
| LSPRWYFYY | | | | 100 |
| ASAFFGMSR | | | | 101 |
| ATEGALNTPK | | | | 102 |
| QLPQGTTLPK | | | | 103 |
| QQQGQTVTK | | | | 104 |
| QQQQGQTVTK | | | | 105 |
| SASAFFGMSR | | | | 106 |
| SQASSRSSSR | | | | 107 |
| TPSGTWLTY | | | | 108 |
| GSFCTQLNR | I | HLA-A*68: 01 | 85.71 | 109 |
| GVVFLHVTY | | | | 110 |
| AQALNTLVK | | | | ill |
| MTSCCSCLK | | | | 112 |
| ASANLAATK | | | | 113 |
| SLIDLQELGK | | | | 114 |
| SVLNDILSR | | | | 115 |
| TQNVLYENQK | | | | 116 |
| CMTSCCSCLK | | | | 117 |
| VQIDRLITGR | | | | 118 |
| KTFPPTEPK | | | | 119 |
| KTFPPTEPKK | | | | 120 |
| LSPRWYFYY | | | | 121 |
| ASAFFGMSR | | | | 122 |
| ATEGALNTPK | | | | 123 |
| QLPQGTTLPK | | | | 124 |
| QQQGQTVTK | | | | 125 |
| QQQQGQTVTK | | | | 126 |
| SASAFFGMSR | | | | 127 |
| SQASSRSSSR | | | | 128 |
| TPSGTWLTY | | | | 129 |
| GYQPYRVVVL | I | HLA-A*23: 01 | 87.72 | 130 |
| PYRVVVLSF | | | | 131 |
| LSPRWYFYY | | | | 132 |
| GSFCTQLNR | I | HLA-A*31: 01 | 89.55 | 133 |
| GVVFLHVTY | | | | 134 |
| AQALNTLVK | | | | 135 |
| MTSCCSCLK | | | | 136 |
| ASANLAATK | | | | 137 |
| SLIDLQELGK | | | | 138 |
| SVLNDILSR | | | | 139 |
| TQNVLYENQK | | | | 140 |
| CMTSCCSCLK | | | | 141 |
| VQIDRLITGR | | | | 142 |
| KTFPPTEPK | | | | 143 |
| KTFPPTEPKK | | | | 144 |
| LSPRWYFYY | | | | 145 |
| ASAFFGMSR | | | | 146 |
| ATEGALNTPK | | | | 147 |
| QLPQGTTLPK | | | | 148 |
| QQQGQTVTK | | | | 149 |
| QQQQGQTVTK | | | | 150 |
| SASAFFGMSR | | | | 151 |
| SQASSRSSSR | | | | 152 |
| TPSGTWLTY | | | | 153 |
| FPNITNLCPF | I | HLA-B*07: 02 | 90.89 | 154 |
| APHGVVFLHV | | | | 155 |
| FPRGQGVPI | | | | 156 |
| APSASAFFGM | | | | 157 |
| GAALQIPFAMQMAYR | II | HLA-DRB1*01: | 91.94 | 158 |
| GWTFGAGAALQIPFA | | 01 | | 159 |
| IDRLITGRLQSLQTY | | | | 160 |
| JSGINASVVNIQKEI | | | | 161 |
| LDKYFKNHTSPDVDL | | | | 162 |
| LGDISGINASVVNIQ | | | | 163 |
| LGFIAGLIAIVMVTI | | | | 164 |
| LNTLVKQLSSNFGAI | | | | 165 |
| LQDVVNQNAQALNTL | | | | 166 |
| LQSLQTYVTQQLIRA | | | | 167 |
| LQTYVTQQLIRAAEI | | | | 168 |
| AQKFNGLTVLPPLLT | | | | 169 |
| PCSFGGVSVITPGTN | | | | 170 |
| QIPFAMQMAYRFNGI | | | | 171 |
| QQLIRAAEIRASANL | | | | 172 |
| QTYVTQQLIRAAEIR | | | | 173 |
| AYRFNGIGVTQNVLY | | | | 174 |
| SSNFGAISSVLNDIL | | | | 175 |
| TGRLQSLQTYVTQQL | | | | 176 |
| WLGFIAGLIAIVMVT | | | | 177 |
| CVNFNFNGLTGTGVL | | | | 178 |
| DKYFKNHTSPDVDLG | | | | 179 |
| IDAYKTFPPTEPKKD | | | | 180 |
| MSRIGMEVTPSGTWL | | | | 181 |
| NKHIDAYKTFPPTEP | | | | 182 |
| VLQLPQGTTLPKGFY | | | | 183 |
| FPRGQGVPI | | HLA-8*08: 01 | 92.85 | 184 |
| FPNITNLCPF | | HLA-8*35: 01 | 93.53 | 185 |
| APHGVVFLHV | | | | 186 |
| FPRGQGVPI | | | | 187 |
| APSASAFFGM | | | | 188 |
| LQIPFAMQM | | HLA-8*15: 01 | 94.18 | 189 |
| RVDFCGKGY | | | | |
| FPNITNLCPF | | HLA-8*51: 01 | 94.72 | 190 |
| APHGVVFLHV | | | | 191 |
| FPRGQGVPI | | | | 192 |
| APSASAFFGM | | | | 193 |
| YEQYIKWPWY | | HLA-8*18: 01 | 95.23 | 194 |
| GRLQSLQTY | | HLA-8*27: 05 | 95.55 | 195 |
| RVDFCGKGY | | | | 196 |
| VRFPNITNL | | | | 197 |
| MTSCCSCLK | | HLA-A*33: 01 | 95.79 | 198 |
| SLIDLQELGK | | | | 199 |
| CMTSCCSCLK | | | | 200 |
| VQIDRLITGR | | | | 201 |
| SASAFFGMSR | | | | 202 |
| SQASSRSSSR | | | | 203 |
| LQIPFAMQM | | HLA-8*58: 01 | 95.99 | 204 |
| RVDFCGKGY | | | | 205 |
| LQIPFAMQM | | HLA-C*15: 02 | 96.17 | 206 |
| RVDFCGKGY | | | | 207 |
| VRFPNITNL | | HLA-C*14: 02 | 96.29 | 208 |

Of the various epitopes evaluated, three epitopes (QPYRVVVLSF (SEQ ID NO:209), GYQPYRVVVL (SEQ ID NO:59), and PYRVVVLSF (SEQ ID NO:60)), are located entirely in the SARS-CoV receptor-binding motif www.uniprot.org/uniprotUP59594), known to be important for virus cell entry and are presumably good immunogenic candidates.

Similar to T cell epitopes, Ahmed et al. (supra) identified SARS-CoV-derived B cell epitopes that have been experimentally-determined from positive B cell assays and identified 49 epitope-sequences, all derived from structural proteins, have an identical match and comprised no mutation in the available SARS-CoV-2 protein sequences (as of 21 Feb. 2020) (see, e.g., Table 4). In certain embodiments the immunogenic nanoparticles described herein comprise one or more of the S-protein epitopes and/or one or more of the N-protein epitopes shown in Table 4. In certain embodiments the immunogenic nanoparticles described herein comprise at least two of the epitopes listed in Table 4. In certain embodiments the immunogenic nanoparticles described herein comprise at least three of the epitopes listed in Table 4. In certain embodiments the immunogenic nanoparticles described herein comprise at least four of the epitopes listed in Table 4. In certain embodiments the immunogenic nanoparticles described herein comprise at least five of the epitopes listed in Table 4.

TABLE 4
SARS-CoV-derived linear B cell epitopes
from S (23; 20 of which are located in
subunit S2) and N (22) proteins that are
identical in SARS-CoV-2 (45 epitopes in total).

| | | IEDB | | SEQ |
|---|---|---|---|---|
| Protein | Subunit | ID | Epitope | ID NO |
| S | S2 | 10778 | DVVNQNAQALNTLVKQL | 210 |
| S | S2 | 11038 | EAEVQIDRLITGRLQSL | 211 |
| S | S2 | 12426 | EIDRLNEVAKNLNESLI | 212 |
| | | | DLQELGKYEQY | |
| S | S2 | 14626 | EVAKNLNESLIDLQELG | 213 |
| S | S2 | 18515 | GAALQIPFAMQMAYRFN | 214 |
| S | S1 | 18594 | GAGICASY | 215 |
| S | S2 | 2092 | AISSVLNDILSRLDKVE | 216 |
| S | S2 | 22321 | GSFCTQLN | 217 |
| S | S2 | 27357 | ILSRLDKVEAEVQIDRL | 218 |
| S | S1 | 30987 | KGIYQTSN | 219 |
| S | S2 | 3176 | AMQMAYRF | 220 |
| S | S2 | 32508 | KNHTSPDVDLGDISGIN | 221 |
| S | S2 | 41177 | MAYRFNGIGVTQNVLYE | 222 |
| S | S2 | 462 | AATKMSECVLGQSKRVD | 223 |
| S | S2 | 47479 | PFAMQMAYRFNGJGVTQ | 224 |
| S | S2 | 50311 | QALNTLVKQLSSNFGAI | 225 |
| S | S2 | 51379 | QLIRAAEIRASANLAAT | 226 |
| S | S1 | 52020 | QQFGRD | 227 |
| S | S2 | 53202 | RASANLAATKMSECVLG | 228 |
| S | S2 | 54599 | RLITGRLQSLQTYVTQQ | 229 |
| S | S2 | 558417 | EIDRLNEVAKNLNESLI | 230 |
| | | | DLQELGKYEQY | |
| S | S2 | 59425 | SLQTYVTQQLIRAAEIR | 231 |
| S | S2 | 9094 | DLGDJSGINASVVNJQK | 232 |
| N | | 15814 | FFGMSRIGMEVTPSGTW | 233 |
| N | | 21065 | GLPNNTASWFTALTQHG | 234 |
| | | | K | |
| N | | 22855 | GTILPK | 235 |
| N | | 28371 | IRQGTDYKHWPQIAQFA | 236 |
| N | | 31116 | KHIDAYKTFPPTEPKKD | 237 |
| | | | KKK | |
| N | | 31166 | KHWPQIAQFAPSASAFF | 238 |
| N | | 75235 | YNVTQAFGRRGPEQTQG | 239 |
| | | | NF | |
| N | | 33669 | KTFPPTEPKKDKKKK | 240 |
| N | | 37640 | LLPAAD | 241 |
| N | | 38249 | LNKHIDAYKTFPPTEPK | 242 |
| N | | 38648 | LPQGTILPKG | 243 |
| N | | 38657 | LPQRQKKQ | 244 |
| N | | 48067 | PKGFYAEGSRGGSQASS | 245 |
| | | | R | |
| N | | 50741 | QFAPSASAFFGMSRIGM | 246 |
| N | | 50965 | QGTDYKHW | 247 |
| N | | 51483 | QLPQGTILPKGFYAE | 248 |
| N | | 51484 | QLPQGTILPKGFYAEGS | 249 |
| | | | R | |
| N | | 51485 | QLPQGTTLPKGFYAEGS | 250 |
| | | | RGGSQ | |
| N | | 63729 | TFPPTEPK | 251 |
| N | | 55683 | RRPQGLPNNTASWFT | 252 |
| N | | 60379 | SQASSRSS | 253 |
| N | | 60669 | SRGGSQASSRSSSRSR | 254 |

Other approaches have been taken to identify SARS-CoV-2 B cell and T cell epitopes. For example, Grifoni et al. (2020) Cell Host Microbe, 27(4): 671-680, used the Immune Epitope Database and Analysis Resource (IEDB) to catalog available data related to other coronaviruses. This includes SARS-CoV, which has high sequence similarity to SARS-CoV-2 and is the best-characterized coronavirus in terms of epitope responses. The identified multiple specific regions in SARS-CoV-2 that have high homology to the SARS-CoV virus and parallel bioinformatic predictions identified a priori potential B and T cell epitopes for SARS-CoV-2. It is believed these predictions provide suitable targets for immune recognition of SARS-CoV-2. These predictions can facilitate effective vaccine design against this virus of high priority.

In particular, to define potential B cell epitopes, using used the predictive tools provided with the IEDB Analysis Resource, B cell epitope predictions were carried out using the SARS-CoV-2 surface glycoprotein, nucleocapsid phosphoprotein, and membrane glycoprotein sequences, have been found to be the main protein targets for B cell responses to other coronaviruses. Additionally, predictions for linear B cell epitopes were made with Bepipred 2.0 (Jespersen et al. (2017) Nucleic Acids Res. 45: W24-W29), and for conformational epitopes with Discotope 2.0 (Kringelum et al. (2012) PLoS Comput Biol. 8: e1002829). A list of B cell epitopes for SARS-CoV and B cell epitope predictions for SARS-CoV-2 are shown in Table 5. In certain embodiments the immunogenic nanoparticles described herein comprise one epitope listed in Table 5. In certain embodiments the immunogenic nanoparticles described herein comprise at least two of the epitopes listed in Table 5. In certain embodiments the immunogenic nanoparticles described herein comprise at least three of the epitopes listed in Table 5. In certain embodiments the immunogenic nanoparticles described herein comprise at least four of the epitopes listed in Table 5. In certain embodiments the immunogenic nanoparticles described herein comprise at least five of the epitopes listed in Table 5.

TABLE 5
Dominant SARS-CoV and SARS-CoV-2 B cell
epitope regions (see Grifoni et al.
(2020) Cell Host Microbe, 27(4): 671-680).

| SARS-CoV | | SARS-CoV-2 | | |
|---|---|---|---|---|
| | SEQ | | | SEQ |
| | ID | | | ID |
| Epitope Sequence | NO | Epitope Sequence | Protein | NO |
| DAVDCSQNPLAELKCS | 255 | DAVDCALDPLSETKCT | S | 256 |
| VKSFEIDKGIYQTSNF | | LKSFTVEKGIYQTSN | | |
| VCGPKLSTDLIKNQCV | 257 | VCGPKKSTNLVKNKCV | S | 258 |
| NFNFNGLTGTGVLTPS | | NFNFNGLTGTGVLTES | | |
| SKRFQPFQQFGRDVSD | | NKKFLPFQQFGRDIAD | | |
| FTDSVRDPKTSEILDI | | TTDAVRDPQTLEILDI | | |
| SPCSFGGVSVIT | | TPCSFGGVSVIGTNTS | | |
| | | NQVAVLYQDVNCT | | |
| GTNASSEVAVLYQDVN | 259 | EVPVAIHADQLTPTWR | S | 260 |
| CTDVSTAIHADQLTPA | | VYSTGS | | |
| WRIYSTGNN | | | | |
| FSQILPDPLKPTKRSF | 261 | FSQILPDPSKPSKRSF | S | 262 |
| IED | | IE | | |
| FGAGAALQIPFAMQMA | 263 | FGAGAALQIPFAMQMA | S | 264 |
| YRFNGIG | | YRFNGI | | |
| MADNGTITVEELKQLL | 265 | MADSNGTITVEELKKL | M | 266 |
| EQWNLVIG | | LEQWNLVI | | |
| PLMESELVIGAVIIRG | 267 | PLLESELVIGAVILRG | M | 268 |
| HLRMA | | HLRI | | |
| PQGLPNNTASWFTALT | 269 | RPQGLPNNTASWFTAL | N | 270 |
| QHGKEE | | TQHGK | | |
| NNAATVLQLPQGTTLP | 271 | NNNAATVLQLPQGTTL | N | 272 |
| KGFYA | | PKGF | | |
| KHIDAYKTFPPTEPKK | 273 | NKHIDAYKTFPPTEPK | N | 274 |
| DKKKKTDEAQPLPQRQ | | KDKKKKTDEAQPLPQR | | |
| KKQPTVTLLPAADMDD | | QKKQPTVTLLPAADM | | |

S = Surface glycoprotein;
M = Membrane protein;
N = Nucleocapsid protein

To predict CD4 T cell epitopes, Grifoni et al. used the method described by Paul et al. (Paul et al. (2015) J. Immunol. 194: 6164-6176) as implemented in the Tepitool resource in IEDB (Paul et al. (2016) Curr. Protoc. Immunol. 114: 18.19.1-18.19.24). A list of B cell epitopes for SARS-CoV and B cell epitope predictions for SARS-CoV-2 are shown in Table 6. In certain embodiments the immunogenic nanoparticles described herein comprise one epitope listed in Table 6. In certain embodiments the immunogenic nanoparticles described herein comprise at least two of the epitopes listed in Table 6. In certain embodiments the immunogenic nanoparticles described herein comprise at least three of the epitopes listed in Table 6. In certain embodiments the immunogenic nanoparticles described herein comprise at least four of the epitopes listed in Table 6. In certain embodiments the immunogenic nanoparticles described herein comprise at least five of the epitopes listed in Table 6.

TABLE 6
Dominant SARS-CoV and SARS-CoV-2 T cell
epitope regions (see Grifoni et al.
(2020) Cell Host Microbe, 27(4): 671-680).

| SARS-CoV | | SARS-CoV-2 | | |
|---|---|---|---|---|
| | SEQ | | | SEQ |
| | ID | | | ID |
| Epitope Sequence | NO | Epitope Sequence | Protein | NO |
| VRGWVFGSTMNNKSQS | 275 | IRGWIFGTTLDSKTQS | S | 276 |
| VI | | LL | | |
| CTFEYISDAFSLD | 277 | CTFEYVSQPFLMD | S | 278 |
| DAFSLDVSEKSGN | 279 | QPFLMDLEGKQGN | S | 280 |
| TNFRAILTAFSPAQDI | 281 | TRFQTLLALHRSYLTP | S | 282 |
| W | | GDSSSGW | | |
| KSFEIDKGIYQTSNFR | 283 | KSFTVEKGIYQTSNFR | S | 284 |
| VV | | VQ | | |
| STFFSTFKCYGVSATK | 285 | SASFSTFKCYGVSPTK | S | 286 |
| L | | L | | |
| KLPDDFMGCV | 287 | KLPDDFTGCV | S | 288 |
| NIDATSTGNYNYKYRY | 289 | NLDSKVGGNYNYLYRL | S | 290 |
| LR | | FR | | |
| YLRHGKLRPFERDISN | 291 | YLYRLFRKSNLKPFER | S | 292 |
| VP | | DI | | |
| RPFERDISNVPFS | 293 | KPFERDISTEIYQ | S | 294 |
| KSIVAYTMSLGADSSI | 295 | QSIIAYTMSLGAENSV | S | 296 |
| AY | | AY | | |
| SIVAYTMSL | 297 | SIIAYTMSL | S | 298 |
| TECANLLLQYGSFCTQ | 299 | TECSNLLLQYGSFCTQ | S | 300 |
| L | | L | | |
| VKQMYKTPTLKYFGGF | 301 | VKQIYKTPPIKDFGGF | S | 302 |
| NF | | NF | | |
| ESLTTTSTALGKLQDV | 303 | DSLSSTASALGKLQDV | S | 304 |
| V | | V | | |
| ALNTLVKQL | 305 | ALNTLVKQL | S | 306 |
| VLNDILSRL | 307 | VLNDILSRL | S | 308 |
| LITGRLQSL | 309 | LITGRLQSL | S | 310 |
| QLIRAAEIRASANLAA | 311 | QLIRAAEIRASANLAA | S | 312 |
| TK | | TK | | |
| SWFITQRNFFSPQII | 313 | HWFVTQRNFYEPQII | S | 314 |
| RLNEVAKNL | 315 | RLNEVAKNL | S | 316 |
| NLNESLIDL | 317 | NLNESLIDL | S | 318 |
| FIAGLIAIV | 319 | FIAGLIAIV | S | 320 |
| RFFTLGSITAQPVKI | 321 | RIFTIGTVTLKQGEI | Orf 3a | 322 |
| SITAQPVKI | 323 | TVTLKQGEI | Orf 3a | 324 |
| TLACFVLAAV | 325 | TLACFVLAAV | M | 326 |
| GLMWLSYFV | 327 | GLMWLSYFI | M | 328 |
| HLRMAGHSL | 329 | HLRIAGHHL | M | 330 |
| ALNTPKDHI | 331 | ALNTPKDHI | N | 332 |
| LQLPQGTTL | 333 | LQLPQGTTL | N | 334 |
| GETALALLLL | 335 | GDAALALLLL | N | 336 |
| LALLLLDRL | 337 | LALLLLDRL | N | 338 |
| LLLDRLNQL | 339 | LLLDRLNQL | N | 340 |
| RLNQLESKV | 341 | RLNQLESKM | N | 342 |
| TKQYNVTQAF | 343 | TKAYNVTQAF | N | 344 |
| GMSRIGMEV | 345 | GMSRIGMEV | N | 346 |
| MEVTPSGTWL | 347 | MEVTPSGTWL | N | 348 |
| QFKDNVILL | 349 | NFKDQVILL | N | 350 |
| CLDAGINYV | 351 | CLEASFNYL | Orf lab | 352 |
| WLMWFIISI | 353 | WLMWLIINL | Orf lab | 354 |
| ILLLDQVLV | 355 | ILLLDQALV | Orf lab | 356 |
| LLCVLAALV | 357 | SACVLAAEC | Orf lab | 358 |
| ALSGVFCGV | 359 | SLPGVFCGV | Orf lab | 360 |
| TLMNVITLV | 361 | TLMNVLTLV | Orf lab | 362 |
| SMWALVISV | 363 | SMWALIISV | Orf lab | 364 |

S = surface glycoprotein;
M = membrane protein;
N = nucleocapsid phosphoprotein.

Accordingly, in various embodiments, immunogenic nanoparticles are contemplated that comprise one or more of the above-identified epitopes (e.g., SEQ ID NOs:1-364) or an immunogenic fragment thereof (e.g., a fragment comprising or consisting of at least 8 contiguous amino acids, or a fragment comprising or consisting of at least 9 contiguous amino acids, or a fragment comprising or consisting of at least 10 contiguous amino acids, or a fragment comprising or consisting of at least 11 contiguous amino acids, or a fragment comprising or consisting of at least 12 contiguous amino acids, or a fragment comprising or consisting of at least 13 contiguous amino acids, or a fragment comprising or consisting of at least 14 contiguous amino acids, or a fragment comprising or consisting of at least 15 contiguous amino acids, where the full length epitope is longer than the stated fragment size).

As explained above, in certain embodiments the nanoparticles comprise peptide comprising multiple epitopes and/or a nucleic acid encoding a peptide comprising multiple epitopes. In certain embodiments the nanoparticles comprise at least 2, or at least 3, or at least 4, or at least 5, or at least 6, or at least 7, or at least 8, or at least 9, or at least 10 different epitopes represented by SEQ ID NOs:1-364, or immunogenic fragment(s) thereof.

It is noted that bioinformatics approaches have been exploited to identify epitopes particularly well suited for incorporation into multiple epitope vaccines for SARS-CoV-2 (see, e.g., Behmard et al. (2020) Sci. Reps., 10: 20864; Yarmarkovich et al. (2020) Cell Reports Med., 1:100036; and the like).

In particular, as described by Behmard et al. immunoinformatics tools were used to design a multi-epitope vaccine polypeptide with the highest potential for activating the human immune system against SARS-CoV-2. The initial epitope set was extracted from the whole set of viral structural proteins. Selected epitopes were bound to each other with appropriate linkers, followed by appending a suitable adjuvant to increase the immunogenicity of the vaccine polypeptide. Cytotoxic T lymphocyte (CTL) epitopes (Table 7) and of helper T lymphocyte (HTL) epitopes (Table 8) selected for multi-epitope vaccine construction were identified.

TABLE 7
Cytotoxic T lymphocyte (CTL) epitopes selected
for multi-epitope vaccine construction (see, e.g.,
Behmard et al. (2020) Sci. Reps., 10: 20864).

| | | SEQ | |
|---|---|---|---|
| | | ID | |
| Protein | Peptide | NO | Allele |
| S | ³⁸6KLNDLCFTNV³⁹⁵ | 365 | HLA-A*02:03; |
| | | | HLA-A*02:01 |
| S | ³²⁹FPNITNLCPF³³⁸ | 366 | HLA-B*53:01; |
| | | | HLA-B*35:01 |
| S | ²⁰⁰FKIYSKHTPI²⁰⁹ | 367 | HLA-A*02:03 |
| S | ¹⁰⁶⁰VVFLHVTYV¹⁰⁶⁸ | 368 | HLA-A*02:03; |
| | | | HLA-A*02:06; |
| | | | HLA-A*02:01 |
| S | ⁵⁸⁷ITPCSFGGV⁵⁹⁵ | 369 | HLA-A*68:02; |
| | | | HLA-A*02:06 |
| S | ¹²⁰⁷EQYIKWPWYI¹²¹⁶ | 370 | HLA-A*23:01; |
| | | | HLA-A*24:02 |
| S | ²⁶⁵YYVGYLQPR²⁷³ | 371 | HLA-A*33:01 |
| S | ²²⁹LPIGINITRF²³⁸ | 372 | HLA-B*53:01; |
| | | | HLA-B*35:01 |
| S | ⁵¹²VLSFELLHA⁵²⁰ | 373 | HLA-A*02:03 |
| S | ⁴⁰⁸RQIAPGQTGK⁴¹⁷ | 374 | HLA-A*03:01 |
| S | ¹¹⁹⁶SLIDLQELGK¹²⁰⁵ | 375 | HLA-A*11:01 |
| S | ⁶⁴⁴QTRAGCLIGA⁶⁵³ | 376 | HLA-A*68:02 |
| E | ⁶¹RVKNLNSSR⁶⁹ | 377 | HLA-A*31:01; |
| | | | HLA-A*30:01 |
| E | ¹⁸LLFLAFVVF²⁶ | 378 | HLA-B*15:01 |
| E | ⁵⁷YVYSRVKNL⁶⁵ | 379 | HLA-A*02:03 |
| E | ²⁹VTLAILTALR³⁸ | 380 | HLA-A*68:01 |
| E | ²³FVVFLLVTL³¹ | 381 | HLA-A*02:06 |
| E | ²⁶FLLVTLAIL³⁴ | 382 | HLA-A*02:01 |
| E | ⁴⁵NIVNVSLVK⁵³ | 383 | HLA-A*68:01 |
| M | ¹⁹QWNLVIGFLF²⁸ | 384 | HLA-A*23:01 |
| M | ¹²IAMACLVGL²⁰ | 385 | HLA-A*68:02 |
| M | ⁶GTITVEELK¹⁴ | 386 | HLA-A*68:01; |
| | | | HLA-A*11:01 |
| M | ³⁵RTRSMWSFNP⁴⁴ | 387 | HLA-A*30:01 |
| M | ⁵¹SGFAAYSRYR⁶⁰ | 388 | HLA-A*31:01 |
| M | ²²LVIGFLFLT³⁰ | 389 | HLA-A*02:06 |
| M | ²⁶FLFLTWICL³⁴ | 390 | HLA-A*02:01 |
| M | ¹⁰IAIAMACLV¹⁸ | 391 | HLA-A*02:06 |
| M | ⁶⁰VTLACFVLAAV⁷⁰ | 392 | HLA-A*02:03 |
| M | ²⁹SFRLFARTR³⁷ | 393 | HLA-A*31:01; |
| | | | HLA-A*33:01 |
| N | ³¹⁵FGMSRIGMEV³²⁴ | 394 | HLA-A*02:03; |
| | | | HLA-A*02:01 |
| N | ³⁶¹KTFPPTEPKK³⁷⁰ | 395 | HLA-A*30:01; |
| | | | HLA-A*11:01 |
| N | ¹⁰⁰KMKDLSPR¹⁰⁷ | 396 | HLA-A*31:01 |
| N | ¹⁹³SSRNSTPGS²⁰¹ | 397 | HLA-A*30:01 |
| N | ¹⁰⁴LSPRWYFYYL¹¹³ | 398 | HLA-B*08:01 |

In certain embodiments, immunogenic nanoparticles comprising one or more, or two or more epitopes from Table 7 (SEQ ID Nos: 365-398) or immunogenic nanoparticles comprising a nucleic acid encoding one or more, or two or more epitopes from Table 7 (SEQ ID Nos: 365-398) are contemplated.

TABLE 8
Illustrative set of helper T lymphocyte (HTL)
epitopes selected for multi-epitope vaccine
construction (see, e.g., Behmard et al. (2020)
Sci. Reps., 10: 20864).

| | | SEQ | | SEQ | |
|---|---|---|---|---|---|
| Pro- | | ID | Core | ID | |
| tein | Peptide | NO | peptide | NO | Allele |
| S | ⁵¹¹VVLSFEL | 399 | FELLHAPAT | 400 | HLA-DRB1*01:01 |
| | LHAPATVC⁵²⁵ | | | | |
| S | ¹⁶⁶CTFEYVS | 401 | EYVSQPFLM | 402 | HLA-DPA1*03:01/ |
| | QPFLMDLE¹⁸⁰ | | | | DPB1*04:02; |
| | | | | | HLA-DPA1*02:01/ |
| | | | | | DPB1*01:01 |
| S | 750SNLLLQY | 403 | LLQYGSFCT | 404 | HLA-DRB1*15:01 |
| | GSFCTQLN764 | | | | |
| S | ¹⁶⁸FEYVSQP | 405 | EYVSQPFLM | 406 | HLA-DPA1*03:01/ |
| | FLMDLEGK¹⁸² | | | | DPB1*04:02 |
| S | ⁷⁵¹NLLLQYG | 407 | LLQYGSFCT | 408 | HLA-DRB1*15:01 |
| | SFCTQLNR⁷⁶⁵ | | | | |
| S | ¹⁴²GVYYHKN | 409 | YHKNNKSWM | 410 | HLA-DRB3*02:02 |
| | NKSWMESE¹⁵⁶ | | | | |
| S | ¹⁴¹LGVYYHK | 411 | YHKNNKSWM | 412 | HLA-DRB3*02:02 |
| | NNKSWMES¹⁵⁵ | | | | |
| S | ¹²¹⁰IKWPWYI | 413 | YIWLGFIAG | 414 | HLA-DPA1*01:03/ |
| | WLGFIAGL¹²²⁴ | | | | DPB1*02:01 |
| S | ¹⁴⁰FLGVYYH | 415 | YHKNNKSWM | 416 | HLA-DRB3*02:02 |
| | KNNKSWME¹⁵⁴ | | | | |
| S | 346RFASVYA | 417 | FASVYAWNR | 418 | HLA-DRB5*01:01 |
| | WNRKRISN360 | | | | |
| S | ⁵⁵FLPFFSNV | 419 | FSNVTWFHA | 420 | HLA-DPA1*02:01/ |
| | TWFHAIH⁶⁹ | | | | DPB1*01:01 |
| S | 166CTFEYVS | 421 | YVSQPFLMD | 422 | HLA-DPA1*01:03/ |
| | QPFLMDLE¹⁸⁰ | | | | DPB1*04:01; |
| | | | | | HLA-DPA1*01:03/ |
| | | | | | DPB1*02:01 |
| N | ⁸³QIGYYRRA | 423 | YRRATRRIR | 424 | HLA-DRB1*11:01; |
| | TRRIRGG⁹⁷ | | | | HLA-DRB5*01:01 |
| N | ³⁰³QIAQFAP | 425 | FAPSASAFF | 426 | HLA-DRB1*09:01 |
| | SASAFFGM³¹⁷ | | | | |

The total of 34 CTL, and 12 HTL epitomic peptides were fused to each other by KK, and GPGPG (SEQ ID NO:427) linkers, respectively, followed by adjoining a single LBL epitope a multi-epitope

In view of this, in certain embodiments, the immunogenic nanoparticle comprising one or more epitopes from Table 7 in combination with one or more epitopes from Table 8 or immunogenic nanoparticles comprising a nucleic acid encoding one or more epitopes from Table 7 in combination with one or more epitopes from Table 8 are contemplated. In certain embodiments the multi-epitope constructs further comprises the peptide DVSLVKPSFYVYSRVK (SEQ ID NO:428) which a linear B lymphocyte (LBL) epitope contained in the E protein. In certain embodiments the immunogenic peptide is fused to β-defensin.

Similarly, Yarmarkovich et al. (2020) Cell Reports Med., 1:100036, identified sixty-five 33-mer peptide sequences (see Table 9) that can be included in multiple-epitope vaccines. These include peptides that are contained within evolutionarily divergent regions of the spike protein reported to increase infectivity through increased binding to the ACE2 receptor and within a newly evolved furin cleavage site thought to increase membrane fusion.

TABLE 9
Epitopes that can be
incorporated into SAR-COV-2 vaccines.

| | | SEQ |
|---|---|---|
| Gene | | ID |
| Position | Epitope | NO: |
| ORF1ab_3619 | IAMSAFAMMFVKHKHAFLCLFLLPSLATVAYFN | 429 |
| ORF1ab_2331 | YILFTRFFYVLGLAAIMQLFFSYFAVHFISNSW | 430 |
| ORF1ab_2354 | FAVHFISNSWLMWLIINLVQMAPISAMVRMYIF | 431 |
| ORF1ab_3057 | VTCLAYYFMRFRRAFGEYSHVVAFNTLLFLMSF | 432 |
| M_87 | LVGLMWLSYFIASFRLFARTRSMWSFNPETNIL | 433 |
| ORF1ab_3123 | LAHIQWMVMFTPLVPFWITIAYIICISTKHFYW | 434 |
| ORF1ab_4978 | TVVIGTSKFYGGWHNMLKTVYSDVENPHLMGWD | 435 |
| ORF1ab_3804 | FRLTLGVYDYLVSTQEFRYMNSQGLLPPKNSID | 436 |
| ORF1ab_3783 | YCFLGYFCTCYFGLFCLLNRYFRLTLGVYDYLV | 437 |
| ORF1ab_5147 | MMILSDDAVVCFNSTYASQGLVASIKNFKSVLY | 438 |
| ORF3a_119 | NFVRIIMRLWLCWKCRSKNPLLYDANYFLCWHT | 439 |
| ORF1ab_6417 | RLYLDAYNMMISAGFSLWVYKQFDTYNLWNTFT | 440 |
| ORF1ab_1799 | QQESPFVMMSAPPAQYELKHGTFTCASEYTGNY | 441 |
| ORF1ab_3196 | DVLLPLTQYNRYLALYNKYKYFSGAMDTTSYRE | 442 |
| ORF1ab_6658 | RSQMEIDFLELAMDEFIERYKLEGYAFEHIVYG | 443 |
| ORF1ab_1624 | DDTLRVEAFEYYHTTDPSFLGRYMSALNHTKKW | 444 |
| S_882 | ITSGWTFGAGAALQIPFAMQMAYRFNGIGVTQN | 445 |
| ORF1ab_2562 | SQLMCQPILLLDQALVSDVGDSAEVAVKMFDAY | 446 |
| ORF1ab_5027 | SLVLARKHTTCCSLSHRFYRLANECAQVLSEMV | 447 |
| ORF1ab_5028 | LVLARKHTTCCSLSHRFYRLANECAQVLSEMVM | 448 |
| ORF1ab_5029 | VLARKHTTCCSLSHRFYRLANECAQVLSEMVMC | 449 |
| ORF1ab_5974 | MTYRRLISMMGFKMNYQVNGYPNMFITREEAIR | 450 |
| ORF1ab_4100 | QVVDADSKIVQLSEISMDNSPNLAWPLIVTALR | 451 |
| ORF1ab_4622 | GVPVVDSYYSLLMPILTLTRALTAESHVDTDLT | 452 |
| ORF1ab_5258 | AYPLTKHPNQEYADVFHLYLQYIRKLHDELTGH | 453 |
| ORF1ab_2251 | GVLMSNLGMPSYCTGYREGYLNSTNVTIATYCT | 454 |
| ORF1ab_6122 | YFVKIGPERTCCLCDRRATCFSTASDTYACWHH | 455 |
| ORF1ab_6123 | FVKIGPERTCCLCDRRATCFSTASDTYACWHHS | 456 |
| ORF1ab_2183 | TRSTNSRIKASMPTTIAKNTVKSVGKFCLEASF | 457 |
| ORF1ab_5694 | IVVFDEISMATNYDLSVVNARLRAKHYVYIGDP | 458 |
| M_15 | KLLEQWNLVIGFLFLTWICLLQFAYANRNRFLY | 459 |
| M_38 | AYANRNRFLYIIKLIFLWLLWPVTLACFVLAAV | 460 |
| ORF1ab_5304 | TSRYWEPEFYEAMYTPHTVLQAVGACVLCNSQT | 461 |
| S_1205 | KYEQYIKWPWYIWLGFIAGLIAIVMVTIMLCCM | 462 |
| ORF1ab_3732 | SMWALIISVTSNYSGVVTTVMFLARGIVFMCVE | 463 |
| ORF1ab_1245 | ETKFLTENLLLYIDINGNLHPDSATLVSDIDIT | 464 |
| ORF1ab_473 | IAIILASFSASTSAFVETVKGLDYKAFKQIVES | 465 |
| ORF1ab_536 | FASEAARVVRSIFSRTLETAQNSVRVLQKAAIT | 466 |
| ORF1ab_3698 | RTVYDDGARRVWTLMNVLTLVYKVYYGNALDQA | 467 |
| ORF1ab_4241 | MVLGSLAATVRLQAGNATEVPANSTVLSFCAFA | 468 |
| S_252 | GDSSSGWTAGAAAYYVGYLQPRTFLLKYNENGT | 469 |
| ORF1ab_3396 | NFTIKGSFLNGSCGSVGFNIDYDCVSFCYMHHM | 470 |
| ORF1ab_220 | DFIDTKRGVYCCREHEHEIAWYTERSEKSYELQ | 471 |
| ORF1ab_4148 | ALRQMSCAAGTTQTACTDDNALAYYNTTKGGRF | 472 |
| S_339 | GEVFNATRFASVYAWNRKRISNCVADYSVLYNS | 473 |
| ORF1ab_6970 | TEHSWNADLYKLMGHFAWWTAFVTNVNASSSEA | 474 |
| S_673 | SYQTQTNSPRRARSVASQSIIAYTMSLGAENSV | 475 |
| S_129 | KVCEFQFCNDPFLGVYYHKNNKSWMESEFRVYS | 476 |
| ORF3a_33 | ATIPIQASLPFGWLIVGVALLAVFQSASKIITL | 477 |
| ORF7A_67 | CPDGVKHVYQLRARSVSPKLFIRQEEVQELYSP | 478 |
| ORF1ab_3946 | SEFSSLPSYAAFATAQEAYEQAVANGDSEVVLK | 479 |
| ORF1ab_593 | NNLVVMAYITGGVVQLTSQWLTNIFGTVYEKLK | 480 |
| ORF3a_204 | HSYFTSDYYQLYSTQLSTDTGVEHVTFFIYNKI | 481 |
| S_53 | DLFLPFFSNVTWFHAIHVSGTNGTKRFDNPVLP | 482 |
| S_1030 | SECVLGQSKRVDFCGKGYHLMSFPQSAPHGVVF | 483 |
| N_82 | DQIGYYRRATRRIRGGDGKMKDLSPRWYFYYLG | 484 |
| S_762 | QLNRALTGIAVEQDKNTQEVFAQVKQIYKTPPI | 485 |
| S_165 | NCTFEYVSQPFLMDLEGKQGNFKNLREFVFKNI | 486 |
| S_1081 | ICHDGKAHFPREGVFVSNGTHWFVTQRNFYEPQ | 487 |
| N_305 | AQFAPSASAFFGMSRIGMEVTPSGTWLTYTGAI | 488 |
| S_490 | FPLQSYGFQPTNGVGYQPYRVVVLSFELLHAPA | 489 |
| S_806 | LPDPSKPSKRSFIEDLLFNKVTLADAGFIKQYG | 490 |
| S_394 | NVYADSFVIRGDEVRQIAPGQTGKIADYNYKLP | 491 |
| S_462 | KPFERDISTEIYQAGSTPCNGVEGFNCYFPLQS | 492 |
| S_445 | VGGNYNYLYRLFRKSNLKPFERDISTEIYQAGS | 493 |

In view of this, in certain embodiments, the immunogenic nanoparticle comprising one or more epitopes from Table 9 (SEQ ID Nos:429-493) or immunogenic nanoparticles comprising a nucleic acid encoding one or more epitopes from Table 9 are contemplated. In certain embodiments the immunogenic nanoparticle comprises 2 or more, or 3 or more, or 4 or more, or 5 or more epitopes found in Table 9 or any one of Tables 7-9 are contemplated as well as immunogenic nanoparticle comprising a nucleic acid that encodes a peptide that comprises 2 or more, or 3 or more, or 4 or more, or 5 or more epitopes found in Table 9 or any one of Tables 7-9.

The above-identified SARS-associated corona virus protein and epitope sequence are illustrative and non-limiting. Using the teachings described herein, numerous other virus protein and/or virus protein fragments for incorporation in the immunogenic nanoparticles described herein will be available to one of skill in the art.

Other Viral Proteins and Protein Fragments

Viruses Associated with Respiratory Diseases

In various embodiments the immunogenic nanoparticles described herein can comprise virus protein and/or virus protein fragments from numerous other viruses associated with respiratory diseases. Such viruses include but are not limited to human influenza viruses types A and B, respiratory syncytial virus (sin-SISH-uhl), human rhinovirus, and parainfluenza viruses (HPIVs). Suitable viral proteins and viral protein fragments (e.g., epitopes) of these viruses are well known to those of skill in the art.

Viruses Associated with Gastrointestinal Pathology.

Both norovirus and rotavirus are associated with gastrointestinal diseases. Norovirus is the leading cause of gastroenteritis worldwide. Accordingly, in certain embodiments, the immunogenic nanoparticles described herein comprise virus protein and/or virus protein fragments from noroviruses. Such proteins and protein fragments (e.g., epitopes) are well known to those of skill in the art (see, e.g., Koromyslova & Hansman (2017) PLoS Pathogens, doi.org/10.1371/joumal.ppat.1006636; van Loben et al. (2019) Viruses, 11(5): 432; and the like).

Rotavirus is a very contagious virus that causes diarrhea. It's the most common cause of diarrhea in infants and children worldwide, resulting in over 215,000 deaths annually. Numerous T cell and B cell epitopes are known to those of skill in the art (see, e.g., Table 10).

TABLE 10
Illustrative rotavirus T cell and B cell
epitopes (from Jafarpour et al. (2015) Viral
Immunol. 28(6): 325-330.

| | | SEQ |
|---|---|---|
| | Epitope | ID |
| Epitope type | Sequence | NO |
| B cell epitope | DEMVRESQRNG | 494 |
| B cell epitope | IDYFVDFVDNCVC | 495 |
| B cell epitope | KNLNDNACT | 496 |
| B cell epitope | NDNACTEY | 497 |
| B cell epitope | PPIQNTRNVVP | 498 |
| B cell epitope | FPYSKTSLWKEMQ | 499 |
| B cell epitope | NYVETARNTID | 500 |
| B cell epitope | VINNGLPPI | 501 |
| B cell epitope | DIIIRRFKIDFKTLK | 502 |
| B cell epitope | QRNGIAPNIFPYSKTS | 503 |
| B cell epitope | TSLWKEMQYNRDIIIR | 504 |
| B cell epitope | IDFKTLKNLNDNACTE | 505 |
| T cell epitope | YNETARNTIDY | 506 |
| T cell epitope | NTIDYFVDFV | 507 |
| T cell epitope | YFVDFVDNVC | 508 |
| T cell epitope | QRNGIAPNIFPY | 509 |
| T cell epitope | WKEMQYNRDI | 510 |
| T cell epitope | RFKIDFKTL | 511 |
| T cell epitope | TLKNLNDNA | 512 |
| T cell epitope | CTEYINNGLPPI | 513 |
| T cell epitope | GIPPIQNTRNVVPG | 514 |

In certain embodiments the immunogenic nanoparticles described herein comprise one or more of the rotavirus B cell epitopes and or one or more of the T cell epitopes shown in Table 10. These epitopes are illustrative and non-limiting. Using the teaching provided herein numerous immunogenic nanoparticles comprising norovirus and/or rotavirus protein and/or protein fragments (e.g., epitopes) will be available to one of skill in the art.

Viruses Associated with Exanthematous Viral Diseases.

Exanthematous viral diseases include, but are not limited to chickenpox/shingles, measles rubella, smallpox, chikungunya virus infection, and other viral infections that produce skin eruptions.

Chickenpox/Shingles.

Chickenpox and shingles are caused by the virus Varicella zoster. Accordingly, in certain embodiments the viral proteins and/or viral protein fragments used in the immunogenic nanoparticles described herein are Varicella zoster virus proteins (e.g., glycoprotein E) and/or protein fragments. In certain embodiments the immunogenic nanoparticles are capable of raising an immune response directed against a Varicella zoster protein. In certain embodiments the immunogenic nanoparticles are capable of raising an immune response directed against a Varicella zoster virus. In certain embodiments the immune response produces Varicella zoster virus neutralizing antibodies.

Suitable immunogenic Varicella zoster viral proteins and protein fragments are well known to those of skill in the art and can readily be incorporated into the immunogenic nanoparticles described herein..

For example, the envelope proteins of varicella-zoster virus (VZV) are highly immunogenic and one of the most abundant is glycoprotein E (gE). Immunodominant regions of glycoprotein E have been identified. In particular, gE amino acids 1-134 and, to a lesser extent, gE(101-161) have been found to be the most antigenic when tested by Western blotting and ELISA. Pepscan analysis of human sera on overlapping synthetic peptides representing residues 1-135 of gE revealed that the most antigenic region was between residues 50 and 135. Three immunodominant sequences (residues 86-105, 116-135, and, to a lesser extent, 56-75) have been detected using sera from both varicella and zoster patients. The neutralizing monoclonal antibody (MAb) IF-B9 reacted with residues 71-90 (see, e.g,. Fowler et al. (1995) Virology, 214(2):5 31-540.

The foregoing Varicella zoster viral proteins and protein fragments are illustrative and non-limiting. Using the teaching provided herein immunogenic nanoparticles containing numerous other Varicella zoster viral proteins and/or protein fragments will be available to one of skill in the art.

Measles.

Globally eliminating measles using available vaccines is biologically feasible because the measles virus (MV) hemagglutinin (H) protein is antigenically stable. The H protein is responsible for receptor binding, and is the main target of neutralizing antibodies. The immunodominant epitope, known as the hemagglutinating and noose epitope, is located near the receptor-binding site (RBS). The RBS also contains an immunodominant epitope.

In view of this, in certain embodiments the viral proteins and/or viral protein fragments used in the immunogenic nanoparticles described herein are measles virus proteins (e.g., hemagglutinin (H) protein) and/or protein fragments. In certain embodiments the immunogenic nanoparticles are capable of raising an immune response directed against a measles protein. In certain embodiments the immunogenic nanoparticles are capable of raising an immune response directed against a measles virus. In certain embodiments the immune response produces measles virus neutralizing antibodies.

Neutralizing antigenic sites of measles virus are well known to those of skill in the art. For example neutralization antigenic sites for hepatitis A have been mapped to a

TABLE 11
Illustrative neutralizing epitopes for measles virus (see,
e.g., Tahara et al. (2016) Viruses, 8(8): 216).

| Viral protein | Amino Acids ¹ |
|---|---|
| β1 | 235 |
| | 233-240 |
| | 235, 244-250 |
| β2 | 302 |
| | 310 |
| | 310 |
| | 309-318 |
| | n.d. ² |
| | 311 |
| β3 | 377-378 |
| | 391 |
| | 391 |
| | 379-400 |
| | 395, 398 |
| | n.d. |
| | n.d. |
| β4 | 473-477 |
| | 491 |
| | n.d. |
| | 488 |
| | 483 |
| β5 | 505, 541, 543, 533 |
| | 505, 506 |
| | 533 |
| | 532, 533 |
| | 547, 546 |
| | 552 |
| β6 | 187 |
| | 190 |
| | 190-200, 571-579 |

The foregoing measles viral proteins and protein fragments are illustrative and non-limiting. Using the teaching provided herein immunogenic nanoparticles containing numerous other measles viral proteins and/or protein fragments will be available to one of skill in the art.

Smallpox

Smallpox was an infectious disease caused by one of two virus variants, Variola major and Variola minor. The last naturally occurring case was diagnosed in October 1977, and the World Health Organization (WHO) certified the global eradication of the disease in 1980. The risk of death following contracting the disease was about 30%, with higher rates among babies. Often those who survived had extensive scarring of their skin, and some were left blind.

While smallpox has substantially been eradicated, concern regarding the use of smallpox as a biowarfare agent has produced a resurgence in various smallpox vaccination options.

In view of this, in certain embodiments the viral proteins and/or viral protein fragments used in the immunogenic nanoparticles described herein are smallpox virus proteins and/or protein fragments. In certain embodiments the immunogenic nanoparticles are capable of raising an immune response directed against a smallpox viral protein. In certain embodiments the immunogenic nanoparticles are capable of raising an immune response directed against a smallpox virus. In certain embodiments the immune response produces smallpox neutralizing antibodies.

Neutralizing antigenic sites of smallpox viruses are well known to those of skill in the art. For example, it has been reported that human leukocyte antigen (HLA) class I restricted T cell epitopes can be recognized more than 30 years after vaccination. Eight epitopes confirmed to stimulate IFN-γ release by T cells in smallpox vaccinated subjects are restricted by five supertypes (HLA-A1, -A2, -A24-A26 and -B44) (see, e.g., Crotty et al. (2003) J. Immunol. 171(10): 4969-4973).

Hepatitis Viruses

Hepatitis, a general term referring to inflammation of the liver, may result from various causes, both infectious (i.e., viral, bacterial, fungal, and parasitic organisms) and noninfectious (e.g., alcohol, drugs, autoimmune diseases, and metabolic diseases). Viral hepatitis accounts for more than 50% of cases of acute hepatitis in the United States, primarily in the emergency department setting.

In the United States, viral hepatitis is most commonly caused by hepatitis A virus (HAV), hepatitis B virus (HBV), and hepatitis C virus (HCV). These three viruses can all result in acute disease with symptoms of nausea, abdominal pain, fatigue, malaise, and jaundice. Additionally, acute infection with HBV and HCV can lead to chronic infection. Patients who are chronically infected may go on to develop cirrhosis and hepatocellular carcinoma (HCC). Furthermore, chronic hepatitis carriers remain infectious and may transmit the disease for many years.

Other hepatotropic viruses known to cause hepatitis include hepatitis D virus (HDV) and hepatitis E virus (HEV). Infections with hepatitis viruses, especially HBV and HBC, have been associated with a wide variety of extrahepatic manifestations.

In view of this, in certain embodiments the viral proteins and/or viral protein fragments used in the immunogenic nanoparticles described herein are hepatitis virus (e.g., hepatitis A, and/or hepatitis B, and/or hepatitis C, and/or hepatitis DA, and/or hepatitis E) proteins and/or protein fragments. In certain embodiments the immunogenic nanoparticles are capable of raising an immune response directed against a hepatitis protein. In certain embodiments the immunogenic nanoparticles are capable of raising an immune response directed against a hepatitis virus (e.g., hepatitis A, and/or hepatitis B, and/or hepatitis C, and/or hepatitis DA, and/or hepatitis E). In certain embodiments the immune response produces hepatitis A, and/or hepatitis B, and/or hepatitis C, and/or hepatitis DA, and/or hepatitis E neutralizing antibodies.

Neutralizing antigenic sites of hepatitis viruses are well known to those of skill in the art. For example neutralization antigenic sites for hepatitis A have been mapped to a domain spanning the junction of VP1 and P2A proteins. One domain is located within the VP2 protein at position 57-90 aa. A second domain, located at position 767-842 aa, contains the C-terminal part of the VP1 protein and the entire P2A protein. A third domain, located at position 1403-1456 aa, comprises the C-terminal part of the P2C protein and the N-terminal half of the P3A protein. A fourth domain, located at position 1500-1519 aa, includes almost the entire P3B. Still another domain, located at position 1719-1764 aa, contains the C-terminal region of the P3C protein and the N-terminal region of the P3D protein (see, e.g., Khudyakov et al. (1999) Virology, 260(2): 260-372).

In hepatitis B, epitopes on the preS1 which induce antibodies that neutralize both ad and ay subtypes of hepatitis B virus (HBV). In certain embodiments antigenic epitopes are located at aa 19-26 and 37-45 of the preS1, respectively (see, e.g., Maeng et al. (2000) Virol. 270(1): 9-16).

The hepatitis C virus (HCV) E2 glycoprotein is a major target of the neutralizing antibody response, with multiple type-specific and broadly neutralizing antibody epitopes identified. In particular, the 412-to-423 region can generate neutralizing antibodies that block interaction with the cell surface receptor CD81, with activity toward multiple HCV genotypes (see, e.g., Gu et al. (2008) J. Virol. 92(9): e02066-17. Other illustrative epitopes include, but are not limited to amino acid residues 412-419 and 434-446, located downstream of the hypervariable region I within the HCV E2 protein (see, e.g., Zhang et al. (2007) Proc. Natl. Acad. Sci. USA, 104 (20) 8449-8454).

The foregoing hepatitis viral proteins and protein fragments are illustrative and non-limiting. Using the teaching provided herein immunogenic nanoparticles containing numerous other hepatitis viral proteins and/or protein fragments will be available to one of skill in the art.

Human Papilloma Virus (HPV)

Cervical cancer is the second most common cause of death in women worldwide after breast cancer (Kim et al. (2014) Clin. Exp. Vaccine Res. 3: 168-175). Strong molecular epidemiological evidence shows that persistent infection with high-risk human papillomavirus (HPV) is the major cause of invasive cervical cancer including condylomata (genital warts) and cervical dysplasia (Jagu et al. (2009) JNCI, 101: 782-792). HPV DNA is detected in more than 99% of all tumors of the uterine cervix. Of more than 40 HPV types that are transmissible through the genital area, types HPV16, HPV18, HPV31, HPV33, HPV35, HPV39, HPV45, HPV51, HPV52, HPV56, HPV58, and HPV59 belong to the group of high-risk type viruses by their malignant properties (Bouvard et al. (2009) Lancet Oncology, 10: 321-322). In this group, HPV16 and HPV18 are together responsible for about 70% of all cervical cancers (see, e.g., Kim & Kim (2017) Arch. Pharm. Res. 40: 1050-1063). HPV16, for example, causes about 46-63% of squamous cell carcinomas in the cervix worldwide (Ringstram et al. (2002) Clin. Cancer Res. 8: 3187-3192) and is the most prevalent HPV type (55.1%) in invasive cervical cancer, HPV18 being the second most prevalent (14.3%). Therefore, HPV types 16 and 18 typically principal targets for vaccine development.

In view of this, in certain embodiments the viral proteins and/or viral protein fragments used in the immunogenic nanoparticles described herein are human papilloma virus (HPV) proteins and/or protein fragments. In certain embodiments the immunogenic nanoparticles are capable of raising an immune response directed against an HPVprotein. In certain embodiments the immunogenic nanoparticles are capable of raising an immune response directed against a human papilloma virus (e.g., HPV16, HPV18, etc.). In certain embodiments the immune response produces anti-HPV neutralizing antibodies.

Neutralization antigenic sites of HPV are well known to those of skill in the art. For example neutralization antigenic sites have been mapped to the E6 and E7 proteins. Illustrative B cell and T cell epitopes for HPV16 and HPV18 are shown in Table 12.

TABLE 12
Illustrative human papilloma virus (HPV)
epitopes (see, e.g., Jabbar et al. (2018)
Front. Immunol., Art. 3000).

| | | T and/or | SEQ |
|---|---|---|---|
| | Epitope | B cell | ID |
| Viral protein | Sequence | epitope | NO |
| E6 protein of HPV 16 | LPQLCTE | B cell | 515 |
| | DIILECVYCKQQ | B cell | 516 |
| | RDLCIVY | B cell | 517 |
| | YAVCDKCLKFY | B cell | 518 |
| | RHYCYSL | B cell | 519 |
| | KPLCDLLIRCIN | B cell | 520 |
| | CQKPLCPE | | |
| | MSCCRS | B cell | 521 |
| E6 protein of HPV 16 | DLYCYEQ | B cell | 522 |
| | YNIVTFCCKCD | B cell | 523 |
| | TLRLCVQSTH | B cell | 524 |
| | HVDIRTL | B cell | 525 |
| | GIVCPICS | B cell | 526 |
| E6 protein of HPV 18 | LPDLC | B cell | 527 |
| | EITCVYCKTVLE | B cell | 528 |
| | LTEVVEF | | |
| | KDLFVVYRD | B cell | 529 |
| | HAACHKCIDF | B cell | 530 |
| | YNLLIRCLRCQ | B cell | 531 |
| | GQCHSCCNR | B cell | 532 |
| E7 protein of HPV 18 | QDIVLHL | B cell | 533 |
| | VDLLCHEQ | B cell | 534 |
| | LCMCCKCE | B cell | 535 |
| | RIELVVES | B cell | 536 |
| | FQQL | B cell | 537 |
| | LSFVCPWCA | B cell | 538 |
| E6 Protein peptides | CYSLYGTTL | T cell | 539 |
| from HPV type 16 | VYDFAFRDL | T cell | 540 |
| binding to MHC-I | EYRHYCYSL | T cell | 541 |
| supertype HLA-A*24 | DFAFRDLCI | T cell | 542 |
| | PYAVCDKCL | T cell | 543 |
| | MHQKRTAMF | T cell | 544 |
| | VYCKQQLLR | T cell | 545 |
| E7 protein peptides | QAEPDRAHY | T cell | 546 |
| from HPV type 16 | TTDLYCYEQ | T cell | 547 |
| binding to MHC-I | LQPETTDLY | T cell | 548 |
| supertype A*01 | | | |
| E6 protein peptides | KLPDLCTEL | T cell | 549 |
| from HPV type 18 | FAFKDLFVV | T cell | 550 |
| binding to MHC-I | TVLELTEVV | T cell | 551 |
| supertype A*02 | LQDIEITCV | T cell | 552 |
| | KTVLELTEV | T cell | 553 |
| E7 protein peptides | AEPQRHTML | T cell | 554 |
| from HPV type 18 | LEPQNEIPV | T cell | 555 |
| binding to MHC-1 | CEARIELVV | T cell | 556 |
| supertype B*44 | FQQLFLNTL | T cell | 557 |
| | NEIPVDLLC | T cell | 558 |

The foregoing HPV viral proteins and protein fragments are illustrative and non-limiting. Using the teaching provided herein immunogenic nanoparticles containing numerous other HPV viral proteins and/or protein fragments will be available to one of skill in the art.

Herpes Virus

Herpes simplex virus type 2 (HSV-2) mainly causes genital infections (Nahmias et al. (1980) Clinical aspects of infection with herpes simplex viruses 1 and 2, vol. 2, Elsevier, New York, N.Y., USA). HSV-2 glycoproteins are structural components of the virion envelope and have been implicated in virus-induced alterations of mammalian cells (Norrild (1980) Curr. Topics Microbiol. Immunol. 90: 67-106; Blough & Tiffany (1984) Cell membranes and viral envelopes, Academic Press, New York, N.Y., USA). Moreover, HSV-2 glycoproteins are expressed in infected cell plasma membranes and act as major antigenic stimuli for the cellular and humoral responses of a host.

Some virion glycoproteins, such as glycoproteins B (gB2), C (gC2), and E (gE2), have been described (Spear (1976) J. Virol. 17(3): 991-1008). Glycoproteins G (gG2) and I (gI2) and gB2, gC2, and gE2 apparently play major roles in immune responses to HSV-2. Evidence supports the following suggestions: (i) the purified gB2, gC2, gE2, gG2, and gI2 of HSV type 1 (oral, HSV-1) or HSV-2 (genital) stimulated high titers of type-common virus-neutralizing antibodies (Cohen et al. (1978) J. Virol. 27(1): 172-181; Watson et al. (1982) Science, 218(4570): 381-384); (ii) passive immunizations with the monoclonal antibodies against gB2, gC2, gE2, gG2, and gI2 protected mice from the infection using a lethal dose of HSV (Balachandran et al. (1982) Infect. Immun. 37(3): 1132-1137; Eisenberg et al. (1984) J. Virol. 49(1): 265-268; Kapoor et al. (1982) J. Gen. Virol. 60(2): 225-233); (iii) gB2, gC2, gE2, gG2, and gI2 mediated antibody-dependent, complement-mediated cytotoxicity and antibody-dependent, cell-mediated cytotoxicity (Balachandran et al. (1982) Infect. Immun. 37(3): 1132-1137; Norrild et al. (1979) J. Virol. 32(3): 741-748; Rector et al. (1982) Infect. Immun. 38(1): 168-174)]; (iv) the purified gB2, gC2, gE2, gG2, and gI2 were able to protect mice against lethal infection with either HSV-1 or HSV-2.

In view of this, in certain embodiments the viral proteins and/or viral protein fragments used in the immunogenic nanoparticles described herein are herpes simplex proteins and/or protein fragments. In certain embodiments the immunogenic nanoparticles are capable of raising an immune response directed against a herpes simplex protein. In certain embodiments the immunogenic nanoparticles are capable of raising an immune response directed against a herpes simplex virus (e.g., HSV-2). In certain embodiments the immune response produces anti-HSV neutralizing antibodies.

Neutralization antigenic sites of HSV are well known to those of skill in the art. For example neutralization antigenic sites have been mapped to glycoproteins B (gB2), C (gC2), G, (gG2), E (gE2), and I (gI2). Illustrative antigenic epitopes include, but are not limited to gB2₅₈₋₇₅, gB2₄₆₆₋₄₇₃, gB2₄₆₈₋₄₇₅, gB2₃₀₀₋₃₀₆, gC2₃₂₆₋₃₃₅, gC2₉₅₋₁₀₅, gC2₁₆₁₋₁₇₁, gC2₂₁₆₋₂₂₃, gE2₅₁₄₋₅₂₂, gE2₄₇₁₋₄₇₉, gE2₄₈₃₋₄₉₁, gE2₃₈₅₋₃₉₈, gG2₅₂₆₋₅₃₉, gG2₂₈₆₋₂₉₅, gG2₅₇₂₋₅₇₉, gG2₃₅₀₋₃₆₄, gI2₂₀₂₋₂₂₆, gI2₂₃₆₋₂₅₃, gI2₂₈₆₋₂₉₅, gI2₃₁₉₋₃₃₇, and the like. In certain embodiments neutralizing epitopes include, but are not limited to gB2466-473, GC216-223, gE2₄₈₃₋₄₉₁, gG2₅₇₂₋₅₇₉, and gI2₂₈₃₋₂₉₅.

The foregoing HSV viral proteins and protein fragments are illustrative and non-limiting. Using the teaching provided herein immunogenic nanoparticles containing numerous other HSV viral proteins and/or protein fragments will be available to one of skill in the art.

Poliovirus (PV)

In certain embodiments the viral proteins and/or viral protein fragments used in the immunogenic nanoparticles described herein are poliovirus viral proteins and/or protein fragments. In certain embodiments the immunogenic nanoparticles are capable of raising an immune response directed against a poliovirus protein. In certain embodiments the immunogenic nanoparticles are capable of raising an immune response directed against poliovirus. In certain embodiments the immune response produces anti-poliovirus neutralizing antibodies.

Neutralization antigenic sites of poliovirus are well known to those of skill in the art. Illustrative suitable poliovirus proteins include, but are not limited to poliovirus type 1 core protein, P2A, type 1 protease 2C, type 1 VP1, type 3 VP1/2A, type 3 VP3-VP1 capsid protein, type 1 VP3-VP1/2A, type 1 VP4, type 1 capsid protein, type 2 capsid protein strain Sabin, type 1 Core protein 2B, type 1 Core protein 2C, type 3 protease 2C, type 3 strain Sabin VP1 protein, type 2 VP1 protein, type 1 VP1/2A protein, type 1 VP2 protein, type 3 VP3 protein, type 2 VP3-VP1 capsid protein, type 3 VP4 protein, and the like.

Moreover, specific neutralization antigenic sites of poliovirus are well known to those of skill in the art. For example three major neutralization antigenic sites have been mapped to the three major polypeptides, VP1, VP2, and VP3, composing the virion surface. Site 1 (N—AgI) consists of amino acid residues 91 to 101 and 144 of VP1 (hereafter designated 1091 to 1101 and 1144); site 2 (NAgII) consists of residues 221 to 226 of VP1 (1221 to 1226) and 164 to 170 and 270 of VP2 (2164 to 2170 and 2270); and site 3 (N—Ag III) consists of residues 58 to 60, 70 to 73, 76, 77, and 79 of VP3 (3058 to 3060, 3070 to 3073, 3076, 3077, and 3079) and residues 286 to 290 of VP1 (1286 to 1290). An additional site (N—AgIB) consisting of amino acid residues 1096 to 1104 and 1141 to 1152 in VP1 has also been described (see, e.g., Fikore et al. (1997) J. Virol., 71(9): 6905-6912. Illustrative T cell and/or B cell poliovirus (PV) epitopes are shown in Table 13.

TABLE 13
Illustrative poliovirus (PV) epitopes
(see, e.g., Kanduc et al. (2015) J. Immunol.
Res., Article ID 541282, doi.org/
10.1155/2015/541282).

| | | | | T and/ | |
|---|---|---|---|---|---|
| | | | | or | SEQ |
| IEDB | Epitope | PV | | B cell | ID |
| ID^{a} | Sequence | antigen | PV strain | epitope | NO |
| 30661 | KEVPALTA | VP1 | PV3 Sabin | B | 559 |
| | VETGAT | | | | |
| 31814 | KLEFFTYS | VP1 | PV1 Mahoney | T | 560 |
| | RFDMELTF | | | | |
| | VVTAN | | | | |
| 46859 | PALTAVET | VP1 | PV1 Mahoney | B-T | 561 |
| | GATNPL | | | | |
| 48785 | PPGAPVPE | VP1 | PV1 Mahoney | T | 562 |
| | KWDDYTWQ | | | | |
| | TSSNP | | | | |
| 55952 | RSRSESSI | VP1 | PV1 Mahoney | B | 563 |
| | ESF | | | | |
| 58511 | SIFYTYGT | VP1 | PV1 Mahoney | T | 564 |
| | APARISVP | | | | |
| | YVGI | | | | |
| 59797 | SNAYSHFY | VP1 | PV1 Mahoney | T | 565 |
| | DGFSKVPL | | | | |
| | KDQS | | | | |
| 66978 | TVDNSAST | Poly- | PV1 Mahoney | T | 566 |
| | TNKDKLFA | protein | | | |
| | VWK | | | | |
| 71769 | VVNDHNPT | Poly- | PV1 Mahoney | T | 567 |
| | KVTSKIRV | protein | | | |
| | YLKP | | | | |
| 79155 | ALTLSLPK | Poly- | PV3 (P3/LEON/37 | T | 568 |
| | QQDSLPDT | protein | and P3/LEON 12A | | |
| | KA | | (1)B) | | |
| 79160 | ATNPLAPS | Poly- | PV3 (P3/LEON/37 | T | 569 |
| | DTVQTRHV | protein | and P3/LEON 12A | | |
| | VQ | | (1)B) | | |
| 79186 | DNEQPTTR | Poly- | PV3 (P3/LEON/37 | T | 570 |
| | AQKLFAM | protein | and P3/LEON 12A | | |
| | | | (1)B) | | |
| 79269 | KEVPALTA | Poly- | PV3 (P3/LEON/37 | T | 571 |
| | VETGATNP | protein | and P3/LEON 12A | | |
| | LA | | (1)B) | | |
| 79272 | KHVRVWCP | Poly- | PV3 (P3/LEON/37 | T | 572 |
| | RPPRAVPY | protein | and P3/LEON 12A | | |
| | YG | | (1)B) | | |
| 79318 | NGHALNQV | Poly- | PV3 (P3/LEON/37 | T | 573 |
| | YQIMYIPP | protein | and P3/LEON 12A | | |
| | GA | | (1)B) | | |
| 79350 | QKLFAMWR | Poly- | PV3 (P3/LEON/37 | T | 574 |
| | ITYKDTV | protein | and P3/LEON 12A | | |
| | | | (1)B) | | |
| 79354 | QPTTRAQK | Poly- | PV3 (P3/LEON/37 | T | 575 |
| | LFAMWRI | protein | and P3/LEON 12A | | |
| | | | (1)B) | | |
| 79433 | TRAQKLFA | Poly- | PV3 (P3/LEON/37 | T | 576 |
| | MWRITYK | protein | and P3/LEON 12A | | |
| | | | (1)B) | | |
| 79434 | TRAQKLFA | Poly- | PV3 (P3/LEON/37 | T | 577 |
| | MWRITYKD | protein | and P3/LEON 12A | | |
| | TV | | (1)B) | | |
| 79435 | TRHVVQRR | Poly- | PV3 (P3/LEON/37 | T | 578 |
| | SRSESTIE | protein | and P3/LEON 12A | | |
| | SF | | (1)B) | | |
| 79436 | TSKVRIYM | Poly- | PV3 (P3/LEON/37 | T | 579 |
| | KPKHVRVW | protein | and P3/LEON 12A | | |
| | | | (1)B) | | |
| 79443 | VAIIEVDN | Poly- | PV3 (P3/LEON/37 | T | 580 |
| | EQPTTRAQ | protein | and P3/LEON 12A | | |
| | KL | | (1)B) | | |
| 79461 | VRVVNDHN | Poly- | PV3 (P3/LEON/37 | T | 581 |
| | PTKVTSKV | protein | and P3/LEON 12A | | |
| | RI | | (1)B) | | |
| 79480 | YIPPGAPT | Poly- | PV3 (P3/LEON/37 | T | 582 |
| | PKSWDDYT | protein | and P3/LEON 12A | | |
| | WQ | | (1)B) | | |
| 80446 | ARGACVTI | VP1 | PV1 Mahoney | B | 583 |
| | MTVDNPA | | | | |
| 81394 | EASGPTHS | VP1 | PV1 Mahoney | B | 584 |
| | KEIPALT | | | | |
| 82831 | GPTHSKEI | VP1 | PV1 Mahoney | B | 585 |
| | PALTAVE | | | | |
| 83234 | HSKEIPAL | VP1 | PV1 Mahoney | B | 586 |
| | TAVETGA | | | | |
| 88446 | SDTVQTRH | VP1 | PV1 Mahoney | B | 587 |
| | VVQHRSR | | | | |
| 88495 | SESSIESF | VP1 | PV1 Mahoney | B | 588 |
| | FARGACV | | | | |
| 99863 | AAPARISV | Poly- | PV3 Sabin | B | 589 |
| | PYVGLA | protein | | | |
| 99886 | AHSKEVPA | Poly- | PV3 Sabin | B | 590 |
| | LTAVET | protein | | | |
| 99901 | ARISVPYV | Poly- | PV3 Sabin | B | 591 |
| | GLANAY | protein | | | |
| 99910 | AYSHFYDG | Poly- | PV3 Sabin | B | 592 |
| | FAKVPL | protein | | | |
| 99933 | DFGVLAVR | Poly- | PV3 Sabin | B | 593 |
| | VVNDHN | protein | | | |
| 99963 | DTVQLRRK | Poly- | PV3 Sabin | B | 594 |
| | LEFFTY | protein | | | |
| 100029 | FAMWRITY | Poly- | PV3 Sabin | B | 595 |
| | KDTVQL | protein | | | |
| 100101 | HFYDGFAK | Poly- | PV3 Sabin | B | 596 |
| | VPLKTD | protein | | | |
| 100117 | HNPTKVTS | Poly- | PV3 Sabin | B | 597 |
| | KVRIYM | protein | | | |
| 100138 | IFYTYGAA | Poly- | PV3 Sabin | B | 598 |
| | PARISV | protein | | | |
| 100244 | LANAYSHF | Poly- | PV3 Sabin | B | 599 |
| | YDGFAK | protein | | | |
| 100349 | NPSIFYTY | Poly- | PV3 Sabin | B | 600 |
| | GAAPAR | protein | | | |
| 100382 | PKHVRVWC | Poly- | PV3 Sabin | B | 601 |
| | PRPPRA | protein | | | |
| 100391 | PSDTVQTR | Poly- | PV3 Sabin | B | 602 |
| | HVVQRR | protein | | | |
| 100425 | QKLFAMWR | Poly- | PV3 Sabin | B | 603 |
| | ITYKDT | protein | | | |
| 100430 | QLRRKLEF | Poly- | PV3 Sabin | B | 604 |
| | FTYSRF | protein | | | |
| 100439 | QPTTRAQK | Poly- | PV3 Sabin | B | 605 |
| | LFAMWR | protein | | | |
| 100482 | RPPRAVPY | Poly- | PV3 Sabin | B | 606 |
| | YGPGVD | protein | | | |
| 100492 | SAMTVDDF | Poly- | PV3 Sabin | B | 607 |
| | GVLAVR | protein | | | |
| 100504 | SEVAQGAL | Poly- | PV3 Sabin | B | 608 |
| | TLSLPK | protein | | | |
| 100536 | SVPYVGLA | Poly- | PV3 Sabin | B | 609 |
| | NAYSHF | protein | | | |
| 100559 | TKVTSKVR | Poly- | PV3 Sabin | B | 610 |
| | IYMKPK | protein | | | |
| 100573 | TRAQKLFA | Poly- | PV3 Sabin | B | 611 |
| | MWRITY | protein | | | |
| 100575 | TSKVRIYM | Poly- | PV3 Sabin | B | 612 |
| | KPKHVR | protein | | | |
| 100576 | TSSNPSIF | Poly- | PV3 Sabin | B | 613 |
| | YTYGAA | protein | | | |
| 100580 | TVDDFGVL | Poly- | PV3 Sabin | B | 614 |
| | AVRVVN | protein | | | |
| 100583 | TVQTRHVV | Poly- | PV3 Sabin | B | 615 |
| | QRRSRS | protein | | | |
| 100585 | TWQTSSNP | Poly- | PV3 Sabin | B | 616 |
| | SIFYTY | protein | | | |
| 100586 | TYGAAPAR | Poly- | PV3 Sabin | B | 617 |
| | ISVPYV | protein | | | |
| 100587 | TYKDTVQL | Poly- | PV3 Sabin | B | 618 |
| | RRKLEF | protein | | | |
| 100613 | VLAVRVVN | Poly- | PV3 Sabin | B | 619 |
| | DHNPTK | protein | | | |
| 100619 | VNDHNPTK | Poly- | PV3 Sabin | B | 620 |
| | VTSKVR | protein | | | |
| 100628 | VRIYMKPK | Poly- | PV3 Sabin | B | 621 |
| | HVRVWC | protein | | | |
| 100630 | VRVVNDHN | Poly- | PV3 Sabin | B | 622 |
| | PTKVTS | protein | | | |
| 100631 | VRVWCPRP | Poly- | PV3 Sabin | B | 623 |
| | PRAVPY | protein | | | |
| 100638 | WCPRPPRA | Poly- | PV3 Sabin | B | 624 |
| | VPYYGP | protein | | | |
| 100644 | WRITYKDT | Poly- | PV3 Sabin | B | 625 |
| | VQLRRK | protein | | | |
| 100667 | YMKPKHVR | Poly- | PV3 Sabin | B | 626 |
| | VWCPRP | protein | | | |
| 100672 | YVGLANAY | Poly- | PV3 Sabin | B | 627 |
| | SHFYDG | protein | | | |
| 146178 | AYAPPGAQ | Poly- | PV3 Sabin | B | 628 |
| | PPTSRK | protein | | | |
| 146181 | CGSMMATG | Poly- | PV3 Sabin | B | 629 |
| | KILVAY | protein | | | |
| 146248 | FLFCGSMM | Poly- | PV3 Sabin | B | 630 |
| | ATGKIL | protein | | | |
| 146311 | HQGALGVF | Poly- | PV3 Sabin | B | 631 |
| | AIPEYC | protein | | | |
| 146333 | ILVAYAPP | Poly- | PV3 Sabin | B | 632 |
| | GAQPPT | protein | | | |
| 146390 | KFTFLFCG | Poly- | PV3 Sabin | B | 633 |
| | SMMATG | protein | | | |
| 146494 | PHQIINLR | Poly- | PV3 Sabin | B | 634 |
| | TNNSAT | protein | | | |
| 146496 | PPGAQPPT | Poly- | PV3 Sabin | B | 635 |
| | SRKEAM | protein | | | |
| 146516 | RAVPYYGP | Poly- | PV3 Sabin | B | 636 |
| | GVDYRN | protein | | | |

The foregoing PV viral proteins and protein fragments are illustrative and non-limiting. Using the teaching provided herein immunogenic nanoparticles containing numerous other PV viral proteins and/or protein fragments will be available to one of skill in the art.

In various embodiments, immunogenic nanoparticles are contemplated that comprise one or more of the above-identified epitopes (e.g., SEQ ID NOs:494-636) or an immunogenic fragment thereof (e.g., a fragment comprising or consisting of at least 8 contiguous amino acids, or a fragment comprising or consisting of at least 9 contiguous amino acids, or a fragment comprising or consisting of at least 10 contiguous amino acids, or a fragment comprising or consisting of at least 11 contiguous amino acids, or a fragment comprising or consisting of at least 12 contiguous amino acids, or a fragment comprising or consisting of at least 13 contiguous amino acids, or a fragment comprising or consisting of at least 14 contiguous amino acids, or a fragment comprising or consisting of at least 15 contiguous amino acids, where the full-length epitope is longer than the stated fragment size).

In view of the foregoing numerous viral protein(s) and/or protein fragment(s) for the above-identified viruses and other viruses will be recognized by one of skill in the art and can readily be incorporated into the immunogenic nanoparticles described herein.

### Adjuvants.

In various embodiments the immunogenic nanoparticles described herein may contain one or more adjuvants. Typically the adjuvant(s) are added in order to enhance the immunostimulatory properties of the immunogenic nanoparticles. In this context, an adjuvant may be understood as any compound, that is suitable for incorporation in the immunogenic nanoparticles described herein that, without being bound to a particular theory, initiates, and/or increases an innate immune response (e.g., a viral protein and/or protein fragment) as defined herein. Such innate immune responses strongly influence the adaptive immune response.

In certain embodiments the adjuvant is an adjuvant that elicits a TH1-biased immune response. Without being bound to a particular theory, particularly where the immunogenic nanoparticle comprises coronavirus epitopes, the use of TH1 preferential adjuvants may prevent vaccine-related immunopathology. Such pathology has appeared in previous treatment attempts with SARS-CoV-1, and is thought to originate, inter alia, from the use of TH2-promoting adjuvants. It is noted that in various embodiments, TH1-biased adjuvants can be used for numerous other anti-viral immunogenic nanoparticles.

Without being bound to a particular theory, it is believed the TH1 adjuvant, e.g., beta-inulin or STING, is able to allow the development of a balanced immune response, that reduces the deleterious effects of TH2 skewing. TH2 skewing can lead to eosinophilic lung damage and a type of antibody (IgG1) that enhances rather than neutralizes the viral effect.

In this regard it has been shown that when compared in a murine model a range of recombinant spike protein or inactivated whole-virus vaccine candidates alone or adjuvanted with either alum, CpG, or Advax (a delta inulin-based polysaccharide adjuvant), all vaccines protected against lethal infection. The addition of an adjuvant significantly increased serum neutralizing-antibody titers and reduced lung virus titers on day 3 post-challenge (Okubo et al. (2015) J. Virol., 89(6): 2995-3007). Unadjuvanted or alum-formulated vaccines were associated with significantly increased lung eosinophilic immunopathology on day 6 post-challenge. This was not observed in mice immunized with vaccines formulated with delta inulin adjuvant. Protection against eosinophilic immunopathology by vaccines containing delta inulin adjuvants correlated better with enhanced T-cell gamma interferon (IFN-) recall responses rather than reduced interleukin-4 (IL-4) responses, suggesting that immunopathology predominantly reflects an inadequate vaccine-induced TH1 response (Id.).

In view of these and other observations, it has been proposed that immunization with SARS antigens alone or formulated with alum fails to induce a sufficient number of IFN-secreting memory T cells and this lack of IFN- is then further exacerbated by active TH1 pathway downregulation by the SARS-CoV itself. This enables a cycle of ever-increasing TH2-polarization of the anti-coronavirus response to become established. In view of this it is proposed that the ideal coronavirus vaccine needs to induce not only neutralizing antibodies or, at a minimum, memory B cells capable of rapidly producing neutralizing antibody upon virus exposure but also a robust long-lived T-cell IFN-response, thereby preventing any risk of lung eosinophilic immunopathology (Id.). It is believed this is the vaccine function that can be imparted by formulation of the antigen(s) with a TH1-biased adjuvant (e.g., delta inulin, STING, and the like).

Accordingly, as noted above, in various embodiments the immunogenic nanoparticles described herein comprise one or more TH1-biased adjuvants. TH1-biased (TH1-preferential) adjuvants are well known to those of skill in the art. Illustrative TH1-biased adjuvants include, but are not limited to: a combined aluminum salt and TLR4 agonist, rOv-ASP-1 (recombinant Onchocerca volvulus activation associated protein-1 (see, e.g., He et al. (2009) J. Immunol., 182(7): 4005-4016), IC31@ (a two-component adjuvant consisting of the artificial antimicrobial cationic peptide KLK acting as a vehicle and the TLR9-stimulatory oligodeoxynucleotide ODN1 (see, e.g., Schellack et al. (2006) Vaccine, 24: 5461-5472), SPO1 (Yu et al. (2012) Vaccine, 30(36): 5425-5436), CPG oligonucleotide, alum-TLR7 agonist based on a TLR7 agonist (SMIP7.10), selected from a benzonaphthyridines series of TLR7 agonists, adsorbed to Aluminium Hydroxid TLR7 (see, e.g., Buonsanti et al. (2016) Scientific Reports, 6: Article number: 29063), OprI lipoprotein of Pseudomonas aeruginosa (see, e.g., U.S. Patent Pub. No: 2003/0059439 A1), cathelicidin-derived antimicrobial peptides (see, e.g., PCT Patent Pub. No: WO 02/013857), delta inulin (β-D-[2-1]poly(fructo-furanosyl)α-D-glucose), e.g., Advax-1 and Advax-2, and STING agonists.

In certain embodiments the TH1-promoting adjuvant comprises one or more Stimulator of Interferon Genes (STING) agonists. STING agonists are well known to those of skill and include, but are not limited to amidobenzimidazole (diABZI), 3′,5′-Cyclic diadenylic acid sodium salt (c-DI-AMP sodium salt), 3′,5′-Cyclic diguanylic acid sodium salt (c-Di-GMP sodium salt), 2′,3′-Cyclic guanosine monophosphate-adenosine monophosphate sodium salt (2′,3′-cGAMP), 3′,3′-Cyclic guanosine monophosphate-adenosine monophosphate sodium salt (3′,3′-cGAMP), 5,6-Dimethyl-9-oxo-9H-xanthene-4-acetic acid (DMXAA), (DMXAA), CMA, MK-1454, CRD5500, cyclic di-nucleotide compounds as described in U.S. Patent Publication No: 2020/0062798 A1, which is incorporated herein by reference for the STING agonists described therein, tricyclic heteroaryl compounds as described in U.S. Patent Publication No: 2020/0040009 A1, which is incorporated herein by reference for the STING agonists described therein, heteroaryl amide compounds as described in U.S. Patent Publication No: 2020/0039994 A1, which is incorporated herein by reference for the STING agonists described therein, and the like.

In certain embodiments, amidobenzimidazole (diABZI) is a preferred STING (stimulator of interferon genes) agonist for the immunogenic nanoparticles.

There are a large number of other adjuvants available, many of which can be co-encapsulated with the viral protein and/or protein fragments (antigen) in the biocompatible polymer (e.g., PLGA) nanoparticles. Examples for which there are strong experimental support as vaccine components include bisphosphonates (e.g., alendronate), CpG ODNs (TLR9 agonists) (see, e.g., Bode et al. (2011) Expert Rev. Vaccines. 10(4): 499-511), imiquimod-family compounds (TLR7 agonists) (see, e.g., Zhang et al. (2015) Clin. Vacc. Immunol,, 21(4): 5709-579)), lipopolysaccharide-based compounds (TLR4) (see, e.g., Patil et al. (2014) J. Control Release, 174: 51-62). Other possible choices include, but are not limited to LPS-like compounds (e.g., MPLA, AS01, AS02, AS04, etc.), Flagellin, dsRNA-like compounds (e.g., Poly (I:C) and derivatives). It is also possible to target retinoic acid-inducible gene I (RIG-I)-like receptors (RLRs) using poly (I:C) and derivatives.

In certain embodiments the incorporation of any other adjuvant known to a skilled person and suitable for use in combination with one or more of the viral protein(s) or protein fragment(s) in the immunogenic nanoparticles is contemplated. Illustrative adjuvants include, but are not limited to, TDM, MDP, muramyl dipeptide, pluronics, alum solution, aluminum hydroxide, ADJUMER™ (polyphosphazene), aluminium phosphate gel, glucans from algae, algammulin, aluminium hydroxide gel (alum), highly protein-adsorbing aluminium hydroxide gel, low viscosity aluminium hydroxide gel, AF or SPT (emulsion of squalane (5%), Tween 80 (0.2%), Pluronic L121 (1.25%), phosphate-buffered saline, pH 7.4), AVRIDINE™ (propanediamine), BAY R1005™ ((N-(2-deoxy-2-L-leucylamino-b-D-glucopyranosyl)-N-oc-tadecy 1-dodecanoy I-amide hydroacetate), CALCITRI O LTM (1-alpha,25-dihydroxy-vitamin D3), calcium phosphate gel, CAP™ (calcium phosphate nanoparticles), cholera holo-toxin, cholera-toxin-A1-protein-A-D-fragment fusion protein, sub-unit B of the cholera toxin, CRL 1005 (block copolymer P1205), cytokine-containing liposomes, DDA (dimethyldioctadecylammonium bromide), DHEA (dehydroepiandrosterone), DMPC (dimyristoylphosphatidylcholine), DMPG (dimyristoylphosphatidylglycerol), DOC/alum complex (deoxycholic acid sodium salt), Freund's complete adjuvant, Freund's incomplete adjuvant, gamma inulin, Gerbu adjuvant (mixture of: i)N-acetylglucosaminyl-(P1-4)N-acetylmuramyl-L-alanyl-D-glutamine (GMDP), ii) dimethyldioctadecylammonium chloride (DDA), iii) zinc-L-proline salt complex (ZnPro-8), GM-CSF), GMDP (N-acety lglucosaminyl-(b 1-4)-N-acety lmuramy 1-L-alany 1-D-isoglutamine), imiquimod (1-(2-methypropyl)-1H-imidazo[4,5-c]quinoline-4-amine), ImmTher™ (N-acetylglucosaminyl-N-acetylmuramyl-L-Ala-D-isoGlu-L-Ala-glycerol dipalmitate), DRVs (immunoliposomes prepared from dehydration-rehydration vesicles), interferon-gamma, interleukin-I beta, interleukin-2, interleukin-7, interleukin-12, ISCOMS™, ISCOPREP 7.0.3.™, liposomes, LOXORIBINE™ (7-allyl-8-oxoguanosine), LT oral adjuvant (E. coli labile enterotoxin-protoxin), MF59™, (squalene-water emulsion), MONTANIDE ISA 51™ (purified incomplete Freund's adjuvant), MONTANIDE ISA 720™ (metabolisable oil adjuvant), MPL™ (3-Q-desacyl-4′-monophosphoryl lipid A), MTP-PE and MTP-PE liposomes ((N-acetyl-L-alanyl-D-isoglutaminyl-L-alanine-2-(1,2-dipalmitoyl-sn-glycero-3-(hydroxyphosphoryloxy))-ethylamide, monosodium salt), MURAMETIDE™ (Nac-Mur-L-Ala-D-Gln-OCH3), MURAPALMITINE™ and D-MURAPALMITINE™ (Nac-Mur-L-Thr-D-isoGln-sn-glyceroldipalmitoyl), NAGO (neuraminidase-galactose oxidase), protein cochleates (Avanti Polar Lipids, Inc., Alabaster, Ala.), STIMULON™ (QS-21), Quil-A (Quil-A saponin), S-28463 (4-amino-otec-dimethyl-2-ethoxymethyl-1 H-imidazo[4,5c]quinoline-1-ethanol), SAF-1™ (“Syntex adjuvant formulation”), ROBANE@(2,6,10,15,19,23-hexamethyltetracosan and 2,6,10,15,19,23-hexamethyl-2,6,10,14,18,22-tetracosahexane), stearyltyrosine (octadecyltyrosine hydrochloride), THERAMID® (N-acetylglucosaminyl-N-acetylmuramyl-L-Ala-D-isoGlu-L-Ala-dipalmitoxypropylamide), Theronyl-MD P (TERMURTIDE™ or [thr 1]-MDP, N-acetylmuramyl-L-threo-nyl-D-isoglutamine), Ty particles (Ty-VLPs or virus-like particles), plant derived adjuvants, including QS21, Quil A, Iscomatrix, ISCOM, adjuvants suitable for costimulation including ‘Iomatine, Inulin, microbe derived adjuvants, including Romurtide, DETOX, MPL, CWS, Mannose, CpG nucleic acid sequences, CpG7909, ligands of human TLR 1-10, ligands of murine TLR 1-13, ISS-1018, IC31, Imidazoquinolines, Ampligen, Ribi529, IMOxine, IRIVs, VLPs, cholera toxin, heat-labile toxin, Pam3Cys, Flagellin, GPI anchor, LNFPIII/Lewis X, antimicrobial peptides, UC-1V150, RSV fusion protein, cdiGMP, and adjuvants suitable as antagonists including CGRP neuropeptide.

Other illustrative, but non-limiting adjuvants include is the MATRIX™ (Magnusson et al. (2018) Immunol. Res. 66: 224-233) which consists of 40 nm honeycomb-like nanoparticles derived from plant saponins, mixed with cholesterol and a phospholipid, the GSK adjuvant (ASO3) which is comprised of α-squalene and polysorbate 80 in an oil-in water emulsion (Cohet et al. (2019) Vaccine, 37: 3006-3021), the adjuvant (CpG 1018) (Campbell (2017) Meth. Mol. Biol. 1494: 15-27) which consists of a 22-mer oligonucleotide that interacts with TLR9, and the Vaxine adjuvant (Advax) which is a microparticle comprised of delta-inulin polysaccharides (Hayashi et al. (2017) EBioMedicine, 15: 127-136).

In still another illustrative, but non limiting embodiment, β-defensin (GIINTLQKYYCRVRGG RCAVLSCLPKEEQIGKC STRGRKCCRRKK, (SEQ ID NO:637) can be added as an adjuvant to, for example, the amino terminus of the polypeptide using linker (e.g., EAAAK linker, SEQ ID NO:638).

The foregoing adjuvants are illustrative and non-limiting. Using the teaching provided herein, numerous other adjuvants will be available to one of skill in the art in the formulation of the immunogenic nanoparticles.

### Targeting Moieties.

In certain embodiments the immunogenic nanoparticles described herein have one or more targeting moieties attached to the surface where the targeting moieties bind to and/or facilitate uptake by antigen-presenting cells (APCs) (e.g., macrophages, dendritic cells, and B cells).

In certain embodiments the nanoparticles comprise targeting moieties that bind to dendritic cells or other myeloid or lymphoid antigen-presenting cells. Illustrative dendritic cell receptors that are readily targeted include but are not limited to, DEC205, MR, Dectin-1, DC-SIGN, DNGR-1, FcγR, and the like. An illustrative, but non-limiting list of targeting moieties that bind to these receptors is shown in Table 14.

TABLE 14
Receptors that can be targeted on dendritic
cells and illustrative targeting moieties.

| Receptors | Targeting moieties |
|---|---|
| DEC205 | SAG-1 (T. gondii) |
| | HIV-1 gagP24 |
| | Ova |
| | AHc |
| | RSV fusion protein |
| MR | MUC1 |
| | MAA |
| | hCGβ |
| Dectin-1 | Ova |
| | Diphtheria toxin (CRM₁₉₇) |
| DC-SIGN | Ag85B (Mtb) |
| | triMN-LPR |
| DNGR-1 | Ova |
| | Anti-Clec9A |
| | Ova |
| | MUC1 |
| FcγR | MUC1-Tn |
| | E75 (HER-2) |
| | iFT |
| | gp120_{αgal}/p24 |
| | α-gal |

Abbreviations: AHc = recombinant He of Clostridium botulinum neurotoxin serotype A, MAA = melanoma-associated antigens, iFT = inactivated Francisella tularensis, CTB = cholera toxin B subunit.

In certain embodiments the nanoparticles comprise targeting moieties that bind to macrophages. Illustrative macrophage receptors that are readily targeted include but are not limited to sialoadhesin receptors, folate receptors, galactose receptors, mannose receptors, β-glucan receptors, scavenger receptors, tuftsin receptors, and the like. Illustrative, but non-limiting list of targeting moieties that bind to these receptors is shown in Table 15.

TABLE 15
Receptors that can be targeted on
macrophages and illustrative targeting moieties

| Receptors | Targeting moieties |
|---|---|
| sialoadhesin | sialic acid |
| receptors | 9-N-(4H-thieno[3,2-c]chromene- |
| | 2-carbamoyl)-Neu5Acα2-3Galβ1- |
| | 4GlcNAc (TCCNeu5Ac) |
| folate | folic acid and methotrexate |
| receptors | or dendrimers thereof |
| | folate |
| galactose | galactose residues |
| receptors | lactose |
| | low density lipoprotein (LDL) |
| | ovalbumin (OVA) |
| | lactobionic acid |
| mannose | mannose-rich glycoconjugates |
| receptors | mannose |
| | mannan |
| | mannosylated poly(L-lysine) (MPL) |
| β-glucan | zymosan and other β-glucans |
| receptors | glucan |
| scavenger | poly-guanine |
| receptors | apoB protein fragment |
| tuftsin | Tufsin tetrapeptide |
| receptors | (Thr-Lys-Pro-Arg, (SEQ ID NO: 639)) |

With respect to scavenger receptors, it is noted that scavenger receptors are receptors on macrophages and other cells that bind to numerous ligands, such as bacterial cell-wall components, and remove them from the blood. Illustrative scavenger receptors on macrophages include, but are not limited to stabilin-1, stabilin-2, SCARA1 or MSR1, SCARA2 or MARCO, SCARA3, SCARA4 or COLEC12, SCARA5, SCARB1, SCARB2, SCARB3 or CD36, and the like. In certain embodiments the targeting moiety comprises a moiety (e.g., a peptide) that binds to stabilin-1 and/or to stabilin-2.

Ligands that bind to scavenger receptors are well known to those of skill in the art and, as noted above, can readily be incorporated as a targeting moiety into the immunogenic nanoparticles described herein. In certain embodiments, for example, suitable ligand that binds to stabilin-1 and/or to stabilin-2 comprises a fragment of the apoB protein. In certain embodiments, the fragment ranges in length from about 5 up to about 50, or up to about 40, or up to about 30, or up to about 20 amino acids. In certain embodiments the fragment ranges in length from about 5 up to about 20, or up to about 10 amino acids. In certain embodiments the first targeting moiety is a peptide comprising the amino acid sequence RKRGLK (SEQ ID NO:640). In certain embodiments the first targeting moiety is a peptide comprising the amino acid sequence RLYRKRGLK (SEQ ID NO:641). In certain embodiments the first targeting moiety is a peptide comprising the amino acid sequence CGGKLGRKRYLR (SEQ ID NO:642)). In certain embodiments the surface can be coated by sugars such as mannan as well as targeting moieties such as aptamers and the like. Aptamers are oligonucleotide molecules that exhibit 3D structure to allow them to bind to specific target molecules. Aptamers are usually created by selecting them from a random sequence pool but natural aptamers also exist.

The mannose receptor (cluster of differentiation 206, CD206) is a C-type lectin primarily present on the surface of macrophages, immature dendritic cells and liver sinusoidal endothelial cells. Ligands that bind to the mannose receptor are well known to those of skill in the art and can also readily be incorporated as a targeting moiety into the immunogenic nanoparticles described herein. Illustrative, but non-limiting examples of ligands for the mannose receptor include, but are not limited to mannan, mannose, N-acetylglucosamine, and fucose. In certain embodiments the targeting moiety can comprise a mannan (e.g., a mannan ranging from about 35 to about 30 kDa).

In certain embodiments the targeting moiety can target lymph nodes. Illustrative moieties that target lymph nodes include, but are not limited to CpG (see, e.g., Thomas, et al. (2014) Biomaterials, 35(2): 814-824), and the novel amph-CpG family. The introduction of glycosylated components on the particle surface, has also been shown to allow interaction mannose-binding protein, which activates the complement system and can be used, to promote nanoparticle access to germinal centers, where they promote memory B-cell responses (see Tokatlian, et al. (2019). Science, 363, 649-654).

In certain embodiments the targeting moiety can be attached to the nanoparticle by simple adsorption or by non-covalent linkages. Useful non-covalent linkages include, but are not limited to, affinity binding pairs, such as biotin-streptavidin and immunoaffinity, having sufficiently high affinity to maintain the linkage during use. Such non-covalent linkers/linkages are well known to those of skill in the art.

In certain embodiments the targeting moiety can be covalently coupled to the nanoparticle directly or through a linker. The art is also replete with conjugation chemistries useful for covalently linking a targeting moiety to a second moiety (e.g., a biocompatible polymer). Art-recognized covalent coupling techniques are disclosed, for instance, in U.S. Pat. Nos. 5,416,016, 6,335,435, 6,528,631, 6,861,514, 6,919,439, and the like. Other conjugation chemistries are disclosed in U.S. Patent Publication No. 2004/0249178. Still other conjugation chemistries include: p-hydroxy-benzoic acid linkers (see, e.g., Chang-Po et al. (2002) Bioconjugate Chem. 13(3): 525-529), native ligation (see, e.g., Stetsenko et al. (2000) J. Org. Chem. 65: 4900-4908), disulfide bridge conjugates (see, e.g., Oehlke et al. (2002) Eur. J. Biochem. 269: 4025-4032; Rogers et al. (2004) Nucl. Acids Res. 32(22): 6595-6604), maleimide linkers (see, e.g., Zhu et al. (1993) Antisense Res Dev. 3: 265-275), thioester linkers (see, e.g., Ede et al. (1994) Bioconjug. Chem. 5: 373-378), Diels-Alder cycloaddition (see, e.g., Marchan et al. (2006) Nucl. Acids Res. 34(3): e24); U.S. Pat. No. 6,656,730 and the like). For reviews of conjugation chemistries, see also Tung et al. (2000) Bioconjugate Chem. 11: 605-618, Zatsepin et al. (2005) Curr. Pharm. Des. 11(28): 3639-3654, Juliano (2005) Curr. Opin. Mol. Ther. 7(2): 132-136, and the like. While certain of the foregoing chemistries are utilized for nucleic acid-peptide conjugation one of skill will recognize that they can readily be modified for attachment of first and/or second targeting moieties to the nanoparticles.

The foregoing targeting moieties and methods of attachment to the nanoparticle(s) are illustrative and non-limiting. Using the teaching provided herein numerous other targeting moieties and attachment chemistries will be available to one of skill in the art.

### Auxiliary Components.

In certain embodiments the immunogenic nanoparticles described herein can additionally include one or more auxiliary substances in order to facilitate cytosolic release of the cargo from the endolysosomal compartment and/or to increase the immunogenicity or immunostimulatory capacity of the nanoparticle.

Thus, in certain embodiments the nanoparticles described herein can additionally include substances that facilitate cytosolic release of the cargo from the endolysosomal compartment. Such substances include, but are not limited to endo-osmolytic peptides (e.g., MPG, Pep-1 and PPTG1) that destabilizes the endolysosomal membrane. Other endo-osmolytic peptides include peptides derived from antimicrobial peptides (e.g., LL-37, melittin, and bombolitin V, with glutamic acid substituting for all basic residues, see, e.g., Ahmad et al. (2015) Biochimica et Biophysica Acta (BBA)-Biomembranes, 1848(2): 544-553), and the like.

In certain embodiments the immunogenic nanoparticles described herein can additionally include one or more auxiliary substances to increase the immunogenicity or immunostimulatory capacity of the nanoparticle. Depending on the various types of auxiliary substances, various mechanisms can come into consideration in this respect. For example, compounds that permit or induce the maturation of dendritic cells (DCs), for example lipopolysaccharides, TNF-α or CD40 ligand, form a first illustrative class of suitable auxiliary substances. In general, it is possible to use as auxiliary substance any agent that influences the immune system in the manner of a “danger signal” (LPS, GP96, didodecyldimethylammonium bromide (DDAB) (see, e.g., Liu et al. (2018) Mol. Pharmaceutics, 15: 11: 5227-5235, etc.) or cytokines, such as GM-CSF, which allow an immune response to be enhanced and/or influenced in a targeted manner. Illustrative, auxiliary substances include, but are not limited to, cytokines, such as monokines, lymphokines, interleukins or chemokines, that further promote the innate immune response, such as IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-α, IFN-β, IFN-γ, GM-CSF, G-CSF, M-CSF, LT-β or TNF-α, growth factors, such as hGH, and the like. An illustrative, but non-limiting list of cytokines that can be included in the immunogenic nanoparticles is shown in Table 16.

TABLE 16
Illustrative CC, CL, CXC, and CX3C cytokines that can be incorporated
into immunogenic nanoparticles described herein.

| Type | Name | Gene | Other Name(s) | Receptor | Uniprot |
|---|---|---|---|---|---|
| CC | CCL1 | Scya1 | I-309, TCA-3 | CCR8 | |
| CC | CCL2 | Scya2 | MCP-1 | CCR2 | P13500 |
| CC | CCL3 | Scya3 | MIP-1a | CCR1 | P10147 |
| CC | CCL4 | Scya4 | MIP-1β | CCR1, CCR5 | P13236 |
| CC | CCL5 | Scya5 | RANTES | CCR5 | P13501 |
| CC | CCL6 | Scya6 | C10, MRP-2 | CCR1 | P27784 |
| CC | CCL7 | Scya7 | MARC, MCP-3 | CCR2 | P80098 |
| CC | CCL8 | Scya8 | MCP-2 | CCR1, CCR2B, | P80075 |
| | | | | CCR5 | |
| CC | CCL9/CCL10 | Scya9 | MRP-2, CCF18, | CCR1 | P51670 |
| | | | MIP-1? | | |
| CC | CCL11 | Scya11 | Eotaxin | CCR2, CCR3, | P51671 |
| | | | | CCR5 | |
| CC | CCL12 | Scya12 | MCP-5 | | Q62401 |
| CC | CCL13 | Scya13 | MCP-4, NCC-1, | CCR2, CCR3, | Q99616 |
| | | | Ckβ10 | CCR5 | |
| CC | CCL14 | Scya14 | HCC-1, MCIF, | CCR1 | Q16627 |
| | | | Ckβ1, NCC-2, | | |
| | | | CCL | | |
| CC | CCL15 | Scya15 | Leukotactin-1, | CCR1, CCR3 | Q16663 |
| | | | MIP-5, | | |
| | | | HCC-2, NCC-3 | | |
| CC | CCL16 | Scya16 | LEC, NCC-4, LMC, | CCR1, CCR2, | O15467 |
| | | | Ckβ12 | CCR5, | |
| | | | | CCR8 | |
| CC | CCL17 | Scya17 | TARC, dendrokine, | CCR4 | Q92583 |
| | | | ABCD-2 | | |
| CC | CCL18 | Scya18 | PARC, DC-CK1, | | P55774 |
| | | | AMAC-1, Ckβ7, | | |
| | | | MIP-4 | | |
| CC | CCL19 | Scya19 | ELC, Exodus-3, | CCR7 | Q99731 |
| | | | Ckβ11 | | |
| CC | CCL20 | Scya20 | LARC, Exodus-1, | CCR6 | P78556 |
| | | | Ckβ4 | | |
| CC | CCL21 | Scya21 | SLC, 6Ckine, | CCR7 | O00585 |
| | | | Exodus-2, Ckβ9, | | |
| | | | TCA-4 | | |
| CC | CCL22 | Scya22 | MDC, DC/β-CK | CCR4 | O00626 |
| CC | CCL23 | Scya23 | MPIF-1, Ckβ8, | CCR1 | P55773 |
| | | | MIP-3, MPIF-1 | | |
| CC | CCL24 | Scya24 | Eotaxin-2, MPIF-2, | CCR3 | O00175 |
| | | | Ckβ6 | | |
| CC | CCL25 | Scya25 | TECK, Ckβ15 | CCR9 | O15444 |
| CC | CCL26 | Scya26 | Eotaxin-3, MIP-4a, | CCR3 | Q9Y258 |
| | | | IMAC, TSC-1 | | |
| CC | CCL27 | Scya27 | CTACK, ILC, | CCR10 | Q9Y4X3 |
| | | | Eskine, PESKY, | | |
| | | | skinkine | | |
| CC | CCL28 | Scya28 | MEC | CCR3, CCR10 | Q9NRJ3 |
| CXC | CXCL1 | Scyb1 | Gro-a, GRO1, | CXCR2 | P09341 |
| | | | NAP-3, KC | | |
| CXC | CXCL2 | Scyb2 | Gro-β, GRO2, | CXCR2 | P19875 |
| | | | MIP-2a | | |
| CXC | CXCL3 | Scyb3 | GRO3, MIP-2β | CXCR2 | P19876 |
| CXC | CXCL4 | Scyb4 | PF-4 | CXCR3B | P02776 |
| CXC | CXCL5 | Scyb5 | ENA-78 | CXCR2 | P42830 |
| CXC | CXCL6 | Scyb6 | GCP-2 | CXCR1, CXCR2 | P80162 |
| CXC | CXCL7 | Scyb7 | NAP-2, CTAPIII, | | P02775 |
| | | | β-Ta, PEP | | |
| CXC | CXCL8 | Scyb8 | IL-8, NAP-1, | CXCR1, CXCR2 | P10145 |
| | | | MDNCF, GCP-1 | | |
| CXC | CXCL9 | Scyb9 | MIG, CRG-10 | CXCR3 | Q07325 |
| CXC | CXCL10 | Scyb10 | IP-10, CRG-2 | CXCR3 | P02778 |
| CXC | CXCL11 | Scyb11 | I-TAC, β-R1, IP-9 | CXCR3, CXCR7 | O14625 |
| CXC | CXCL12 | Scyb12 | SDF-1, PBSF | CXCR4, CXCR7 | P48061 |
| CXC | CXCL13 | Scyb13 | BCA-1, BLC | CXCR5 | O43927 |
| CXC | CXCL14 | Scyb14 | BRAK, bolekine | | O95715 |
| CXC | CXCL15 | Scyb15 | Lungkine, WECHE | | Q9WVL7 |
| CXC | CXCL16 | Scyb16 | SRPSOX | CXCR6 | Q9H2A7 |
| CXC | CXCL17 | VCC-1 | DMC, VCC-1 | | Q6UXB2 |
| C | XCL1 | Scyc1 | Lymphotactin a, | XCR1 | P47992 |
| | | | SCM-1a, ATAC | | |
| C | XCL2 | Scyc2 | Lymphotactin β, | XCR1 | Q9UBD3 |
| | | | SCM-1β | | |
| CX3C | CX3CL1 | Scyd1 | Fractalkine, | CX3CR1 | P78423 |
| | | | Neurotactin, | | |
| | | | ABCD-3 | | |

The foregoing auxiliary substances are illustrative and non-limiting. Using the teaching provided herein numerous other auxiliary substances known to one of skill in the art can readily be incorporated in the immunogenic nanoparticles described herein.

### Pharmaceutical Formulations.

In various embodiments pharmaceutical formulations (e.g., formulations suitable for vaccination of a mammal) comprising the immunogenic nanoparticles described herein are provided. Such pharmaceutical formulations can be prepared by any method known or hereafter developed in the art of pharmaceutics. In general, such preparatory methods include the step of bringing the active ingredient (e.g., immunogenic nanoparticles) into association with one or more excipients and/or one or more other accessory ingredients, and then, if necessary and/or desirable, shaping and/or packaging the product into a desired single- or multi-dose unit.

In certain embodiments a pharmaceutical composition may be prepared, packaged, and/or sold in bulk, as a single unit dose, and/or as a plurality of single unit doses. As used herein, a “unit dose” is a discrete amount of the pharmaceutical composition comprising a predetermined amount of the active ingredient (immunogenic nanoparticles). The amount of the active ingredient is generally equal to the dosage of the active ingredient which would be administered to a subject and/or a convenient fraction of such a dosage such as, for example, one-half or one-third of such a dosage.

The relative amounts of the active ingredient (immunogenic nanoparticles), the pharmaceutically acceptable excipient(s), and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient (immunogenic nanoparticles).

In certain embodiments the pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21.sup.st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient is at least 95%, 96%, 97%, 98%, 99%, or 100% pure. In some embodiments, the excipient is approved for use in humans and veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the pharmaceutical formulations comprising the immunogenic nanoparticles described herein. Excipients such as cocoa butter and suppository waxes, coloring agents, coating agents, sweetening, flavoring, and perfuming agents can be present in the composition, according to the judgment of the formulator.

Illustrative diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Illustrative granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water-insoluble starch, calcium carboxymethyl cellulose, magnesium aluminum silicate (Veegum), sodium lauryl sulfate, quaternary ammonium compounds, etc., and combinations thereof.

Illustrative surface active agents and/or emulsifiers include, but are not limited to, natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite [aluminum silicate] and Veegum [magnesium aluminum silicate]), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate [TWEEN20®], polyoxyethylene sorbitan [TWEEN60®], polyoxyethylene sorbitan monooleate [TWEEN80®], sorbitan monopalmitate [SPAN40®], sorbitan monostearate [SPAN®60], sorbitan tristearate [SPAN®65], glyceryl monooleate, sorbitan monooleate [SPAN®80]), polyoxyethylene esters (e.g., polyoxyethylene monostearate [MYRJ®45], polyoxyethylene hydrogenated castor oil, polyethoxylated castor oil, polyoxymethylene stearate, and Solutol), sucrose fatty acid esters, polyethylene glycol fatty acid esters (e.g., CREMOPHOR®), polyoxyethylene ethers, (e.g., polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Illustrative binding agents include, but are not limited to, starch (e.g., cornstarch and starch paste); gelatin; sugars (e.g., sucrose, glucose, dextrose, dextrin, molasses, lactose, lactitol, mannitol,); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Illustrative preservatives may include antioxidants, chelating agents, antimicrobial preservatives, antifungal preservatives, alcohol preservatives, acidic preservatives, and other preservatives. Illustrative antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Illustrative chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Illustrative antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Illustrative antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Illustrative alcohol preservatives include, but are not limited to, ethanol, polyethylene glycol, phenol, phenolic compounds, bisphenol, chlorobutanol, hydroxybenzoate, and phenylethyl alcohol. Illustrative acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, Germall 115, Germaben II, Neolone™, Kathon™, and EUXYL®. In certain embodiments, the preservative is an antioxidant. In other embodiments, the preservative is a chelating agent.

Illustrative buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Illustrative lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Illustrative oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Illustrative oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include, but are not limited to, pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, vaccine nanoparticles of the invention are mixed with solubilizing agents such as CREMOPHOR®, alcohols, oils, modified oils, glycols, polysorbates, cyclodextrins, polymers, and combinations thereof.

Injectable formulations, for example, sterile injectable aqueous or oleaginous suspensions may be formulated according to the known art using suitable dispersing or wetting agents and suspending agents. A sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable media prior to use.

Solid dosage forms for oral administration include capsules, tablets, pills, powders, and granules. In such solid dosage forms, the active ingredient is mixed with at least one inert, pharmaceutically acceptable excipient such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polethylene glycols and the like.

In certain embodiments the active ingredients (immunogenic nanoparticles described herein) can be in micro-encapsulated form with one or more excipients as noted above. Solid dosage forms of tablets, dragees, capsules, pills, and granules can be prepared with coatings and shells such as enteric coatings, release controlling coatings and other coatings well known in the pharmaceutical formulating art. In such solid dosage forms, active ingredient may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

In certain embodiments, dosage forms for topical and/or transdermal administration of vaccine nanoparticles may include ointments, pastes, creams, lotions, gels, powders, solutions, sprays, inhalants and/or patches. Generally, active ingredient is admixed under sterile conditions with a pharmaceutically acceptable excipient and/or any needed preservatives and/or buffers as may be required. Additionally, the use of transdermal patches is also contemplated. Such patches often have the added advantage of providing controlled delivery of an active ingredient to the body. Such dosage forms may be prepared, for example, by dissolving and/or dispensing the active ingredient in the proper medium. Alternatively or additionally, the rate may be controlled by either providing a rate controlling membrane and/or by dispersing the active ingredient in a polymer matrix and/or gel.

Suitable devices for use in delivering intradermal pharmaceutical compositions described herein include soluble microneedle array patches, e.g., as described in U.S. Patent Publication Nos: US 2019/0358441 A1, US 2019/0240469 A1, US 2019/0151638 A1, US 2019/0151638 A1, US 2018/0001070 A1, US 2017/0209553 A1, US 2016/0263362 A1, US 2017/0196966 A1, US 2016/0213908 A1, US 2016/0015952 A1, US 2015/0112250 A1, US 2012/0150023, and the like. Also contemplated are short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices that limit the effective penetration length of a needle into the skin, such as those described in PCT publication WO 99/34850 and functional equivalents thereof. Jet injection devices that deliver vaccines to the dermis via a liquid jet injector and/or via a needle that pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/037705 and WO 97/013537, and the like.. Ballistic powder/particle delivery devices that use compressed gas to accelerate vaccine in powder form through the outer layers of the skin to the dermis are suitable. Alternatively or additionally, conventional syringes may be used in the classical mantoux method of intradermal administration.

In certain embodiments formulations suitable for topical administration include, but are not limited to, liquid and/or semi liquid preparations such as liniments, lotions, oil in water and/or water in oil emulsions such as creams, ointments and/or pastes, and/or solutions and/or suspensions. In certain embodiments, topically-administrable formulations may, for example, comprise from about 1% to about 10% (w/w) active ingredient (immunogenic nanoparticles described herein), although the concentration of the active ingredient may be as high as the solubility limit of the active ingredient in the solvent. In certain embodiments, formulations for topical administration may further comprise one or more of the additional ingredients described herein.

In certain embodiments, a pharmaceutical composition comprising the immunogenic nanoparticles described herein may be prepared, packaged, and/or sold in a formulation suitable for pulmonary administration via the buccal cavity. Such a formulation may, for example, comprise particles that comprise or contain the immunogenic nanoparticles described herein. Such compositions are conveniently in the form of dry powders for administration using a device comprising a dry powder reservoir to which a stream of propellant may be directed to disperse the powder and/or using a self-propelling solvent/powder dispensing container such as a device comprising the active ingredient dissolved and/or suspended in a low-boiling propellant in a sealed container.

Low boiling propellants generally include liquid propellants having a boiling point of below 65° F. at atmospheric pressure. In certain embodiments the propellant may constitute 50% to 99.9% (w/w) of the composition, and the active ingredient (immunogenic nanoparticles described herein) may constitute 0.1% to 20% (w/w) of the composition. The propellant may further comprise additional ingredients such as a liquid non-ionic and/or solid anionic surfactant and/or a solid diluent (which may have a particle size of the same order as particles comprising the active ingredient).

In certain embodiments, pharmaceutical compositions comprising the immunogenic nanoparticles described herein formulated for pulmonary delivery may provide the nanoparticles in the form of droplets of a solution and/or suspension. In certain embodiments, such formulations may be prepared, packaged, and/or sold as aqueous and/or dilute alcoholic solutions and/or suspensions, optionally sterile, comprising the active ingredient, and may conveniently be administered using any nebulization and/or atomization device. In certain embodiments, such formulations may further comprise one or more additional ingredients including, but not limited to, a flavoring agent such as saccharin sodium, a volatile oil, a buffering agent, a surface active agent, and/or a preservative such as methylhydroxybenzoate. In certain embodiments, the droplets provided by this route of administration may have an average diameter in the range from about 0.1 μm up to about 200 μm.

In certain embodiments, the formulations described herein as being useful for pulmonary delivery are useful for intranasal delivery of a pharmaceutical composition of the invention. Another illustrative formulation suitable for intranasal administration is a coarse powder comprising the active ingredient and having an average particle from about 0.2 μm to about 500 μm. Such a formulation can be administered in the manner in which snuff is taken, i.e. by rapid inhalation through the nasal passage from a container of the powder held close to the nares.

In certain embodiments, formulations suitable for nasal administration may, for example, comprise from about as little as 0.1% (w/w) and as much as 100% (w/w) of the immunogenic nanoparticles described herein, and may comprise one or more of the additional ingredients described herein. In certain embodiments, a pharmaceutical composition of immunogenic nanoparticles described herein may be prepared, packaged, and/or sold in a formulation suitable for buccal administration. Such formulations may, for example, be in the form of tablets and/or lozenges made using conventional methods, and may, for example, 0.1% to 20% (w/w) immunogenic nanoparticles, the balance comprising an orally dissolvable and/or degradable composition and, optionally, one or more of the additional ingredients described herein. Alternately, in certain embodiments, formulations suitable for buccal administration may comprise a powder and/or an aerosolized and/or atomized solution and/or suspension comprising the immunogenic nanoparticles. In certain embodiments, such powdered, aerosolized, and/or aerosolized formulations, when dispersed, may have an average particle and/or droplet size in the range from about 0.1 μm to about 200 μm, and may further comprise one or more of the additional ingredients described herein.

General considerations in the formulation and/or manufacture of pharmaceutical agents may be found, for example, in Remington: The Science and Practice of Pharmacy 21.sup.st ed., Lippincott Williams & Wilkins, 2005.

### Administration

In some embodiments, a therapeutically effective amount of an immunogenic nanoparticle vaccine is delivered to a patient and/or animal prior to, simultaneously with, and/or after diagnosis with a disease, disorder, and/or condition. In some embodiments, a therapeutic amount of an inventive composition is delivered to a patient and/or animal prior to, simultaneously with, and/or after onset of symptoms of a disease, disorder, and/or condition. In some embodiments, the amount of a vaccine nanoparticle is sufficient to treat, alleviate, ameliorate, relieve, delay onset of, inhibit progression of, reduce severity of, and/or reduce incidence of one or more symptoms or features of the disease, disorder, and/or condition. In some embodiments, the amount of a vaccine nanoparticle is sufficient to elicit a detectable immune response in a subject. In some embodiments, the amount of a vaccine nanoparticle is sufficient to elicit a detectable antibody response in a subject. In some embodiments, the amount of a vaccine nanoparticle is sufficient to elicit a detectable T cell response in a subject. In some embodiments, the amount of a vaccine nanoparticle is sufficient to elicit a detectable antibody and T cell response in a subject. In some embodiments, an advantage of the nanoparticles provided is that the nanoparticles can elicit potent responses with a much lower concentration of antigen than required with a conventional vaccine.

The compositions, according to the methods described herein, can be administered using any amount and any route of administration effective for treatment. The exact amount required will vary from subject to subject, depending on the species, age, and general condition of the subject, the severity of the infection, the particular composition, its mode of administration, its mode of activity, and the like. The compositions comprising the immunogenic nanoparticles described herein are typically formulated in dosage unit form for ease of administration and uniformity of dosage. It will be understood, however, that the total daily usage of the compositions will be decided by the attending physician within the scope of sound medical judgment. The specific therapeutically effective dose (or prophylactically effective dose) level for any particular subject or organism will depend upon a variety of factors including the disorder being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The pharmaceutical compositions comprising the immunogenic nanoparticles described herein may be administered by any route suitable for administering an immunogen. In some embodiments, the pharmaceutical compositions described are administered by a variety of routes, including oral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. Specifically contemplated routes are oral administration, intravenous injection, intramuscular injection, and/or subcutaneous injection. In some embodiments, the nanoparticles are administered parenterally. In some embodiments, the nanoparticles are administered intravenously. In some embodiments, the nanoparticles are administered orally.

In general the most appropriate route of administration will depend upon a variety of factors including the nature of the vaccine nanoparticle (e.g., its stability in the environment of the gastrointestinal tract), the condition of the subject (e.g., whether the subject is able to tolerate oral administration), etc.

In certain embodiments, the immunogenic nanoparticles described herein may be administered in amounts ranging from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. In certain embodiments, the desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

In some embodiments, the present invention encompasses “therapeutic cocktails” comprising populations immunogenic nanoparticles described herein. In some embodiments, all of the nanoparticles within a population of nanoparticles comprise a single species of targeting moiety which can bind to multiple targets (e.g., can bind to both SCS-Mph and FDCs). In some embodiments, different nanoparticles within a population of nanoparticles comprise different targeting moieties, and all of the different targeting moieties can bind to the same target. In some embodiments, different nanoparticles comprise different targeting moieties, and all of the different targeting moieties can bind to different targets. In some embodiments, such different targets may be associated with the same cell type. In some embodiments, such different targets may be associated with different cell types.

### Combination Therapies

It will be appreciated that immunogenic nanoparticles described herein and pharmaceutical compositions comprising such nanoparticles can be employed in combination therapies. The particular combination of therapies (therapeutics or procedures) to employ in a combination regimen will take into account compatibility of the desired therapeutics and/or procedures and the desired therapeutic effect to be achieved. It will be appreciated that the therapies employed may achieve a desired effect for the same purpose (for example, nanoparticle useful for vaccinating against a particular type of viral infection may be administered concurrently with another agent useful for treating the same viral infection), or they may achieve different effects (e.g., control of any adverse effects attributed to the nanoparticle).

In some embodiments, pharmaceutical compositions comprising the immunogenic nanoparticles described herein may be administered either alone or in combination with one or more other therapeutic agents. By “in combination with,” it is not intended to imply that the agents must be administered at the same time and/or formulated for delivery together, although these methods of delivery are within the scope of the invention. The compositions can be administered concurrently with, prior to, or subsequent to, one or more other desired therapeutics or medical procedures. In general, each agent will be administered at a dose and/or on a schedule determined for that agent. Additionally, the delivery of the immunogenic nanoparticles in combination with agents that may improve their bioavailability, reduce and/or modify their metabolism, inhibit their excretion, and/or modify their distribution within the body is contemplated.

The particular combination of therapies (therapeutics and/or procedures) to employ in a combination regimen will take into account compatibility of the desired therapeutics and/or procedures and/or the desired therapeutic effect to be achieved. It will be appreciated that the therapies employed may achieve a desired effect for the same disorder (for example, an immunogenic nanoparticle may be administered concurrently with another therapeutic agent used to treat the same disorder), and/or they may achieve different effects (e.g., control of any adverse effects attributed to the vaccine nanoparticle). In some embodiments, immunogenic nanoparticles described herein are administered with a second therapeutic agent that is approved by the U.S. Food and Drug Administration.

In will further be appreciated that therapeutically active agents utilized in combination may be administered together in a single composition or administered separately in different compositions.

In general, it is expected that agents utilized in combination will be utilized at levels that do not exceed the levels at which they are utilized individually. In some embodiments, the levels utilized in combination will be lower than those utilized individually.

In some embodiments, the immunogenic nanoparticles may be administered in combination with an agent, including, for example, therapeutic, diagnostic, and/or prophylactic agents. Illustrative agents to be delivered include, but are not limited to, small molecules, organometallic compounds, nucleic acids, proteins (including multimeric proteins, protein complexes, etc.), peptides, lipids, carbohydrates, hormones, metals, radioactive elements and compounds, drugs, vaccines, immunological agents, etc., and/or combinations thereof.

In certain embodiments, the immunogenic nanoparticles that delay the onset and/or progression of a particular viral infection may be administered in combination with one or more additional therapeutic agents that treat the symptoms of the viral infection. To give but one example, upon exposure to rabies virus, the immunogenic nanoparticles useful for vaccination against rabies virus may be administered in combination with one or more therapeutic agents useful for treatment of symptoms of rabies virus (e.g., antipsychotic agents useful for treatment of paranoia that is symptomatic of rabies virus infection).

In some embodiments, pharmaceutical compositions comprising the immunogenic nanoparticles described herein comprise less than 50% by weight, less than 40% by weight, less than 30% by weight, less than 20% by weight, less than 15% by weight, less than 10% by weight, less than 5% by weight, less than 1% by weight, or less than 0.5% by weight of an agent to be delivered.

In some embodiments, the immunogenic nanoparticles are administered in combination with an agent comprising one or more small molecules and/or organic compounds with pharmaceutical activity. In some embodiments, the agent is a clinically-used drug. In some embodiments, the drug is an antibiotic, and/or anti-viral agent, anti-HIV agent, anti-parasite agent, anti-protozoal agent, anesthetic, anticoagulant, inhibitor of an enzyme, steroidal agent, steroidal or non-steroidal anti-inflammatory agent, antihistamine, immunosuppressant agent, anti-neoplastic agent, antigen, vaccine, antibody, decongestant, sedative, opioid, analgesic, anti-pyretic, birth control agent, hormone, prostaglandin, progestational agent, anti-glaucoma agent, ophthalmic agent, anti-cholinergic, analgesic, anti-depressant, anti-psychotic, neurotoxin, hypnotic, tranquilizer, anti-convulsant, muscle relaxant, anti-spasmodic, muscle contractant, channel blocker, miotic agent, anti-secretory agent, anti-thrombotic agent, anticoagulant, anti-cholinergic, β-adrenergic blocking agent, diuretic, cardiovascular active agent, vasoactive agent, vasodilating agent, anti-hypertensive agent, angiogenic agent, modulators of cell-extracellular matrix interactions (e.g., cell growth inhibitors and anti-adhesion molecules), inhibitors of DNA, RNA, or protein synthesis, etc.

In certain embodiments, a small molecule agent can be any drug. In some embodiments, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460; drugs for veterinary use are listed by the FDA under 21 C.F.R. §§ 500 through 589. All listed drugs are considered acceptable for use in accordance with the present invention.

A more complete listing of classes and specific drugs suitable for use in the present invention may be found in Pharmaceutical Drugs: Syntheses, Patents, Applications by Axel Kleemann and Jurgen Engel, Thieme Medical Publishing, 1999 and the Merck Index: An Encyclopedia of Chemicals, Drugs and Biologicals, Ed. by Budavari et al., CRC Press, 1996, both of which are incorporated herein by reference.

In some embodiments, the immunogenic nanoparticles are administered in combination with one or more nucleic acids (e.g., functional RNAs, functional DNAs, etc.) to a specific location such as a tissue, cell, or subcellular locale. For example, the immunogenic nanoparticles that are used to delay the onset and/or progression of a particular viral infection may be administered in combination with RNAi agents which reduce expression of viral proteins.

In some embodiments, the immunogenic nanoparticles are administered in combination with one or more proteins or peptides. In some embodiments, the agent to be delivered may be a peptide, hormone, erythropoietin, insulin, cytokine, antigen for vaccination, etc. In some embodiments, the agent to be delivered may be an antibody and/or characteristic portion thereof.

In some embodiments, the immunogenic nanoparticles are administered in combination with one or more carbohydrates, such as a carbohydrate that is associated with a protein (e.g., glycoprotein, proteoglycans, etc.). A carbohydrate may be natural or synthetic. A carbohydrate may also be a derivatized natural carbohydrate. In certain embodiments, a carbohydrate may be a simple or complex sugar. In certain embodiments, a carbohydrate is a monosaccharide, including but not limited to glucose, fructose, galactose, and ribose. In certain embodiments, a carbohydrate is a disaccharide, including but not limited to lactose, sucrose, maltose, trehalose, and cellobiose. In certain embodiments, a carbohydrate is a polysaccharide, including but not limited to cellulose, microcrystalline cellulose, hydroxypropyl methylcellulose (HPMC), methylcellulose (MC), dextrose, dextran, glycogen, xanthan gum, gellan gum, starch, and pullulan. In certain embodiments, a carbohydrate is a sugar alcohol, including but not limited to mannitol, sorbitol, xylitol, erythritol, maltitol, and lactitol.

In some embodiments, the immunogenic nanoparticles are administered in combination with one or more lipids, such as a lipid that is associated with a protein (e.g., lipoprotein). Illustrative lipids that may be used include, but are not limited to, oils, fatty acids, saturated fatty acid, unsaturated fatty acids, essential fatty acids, cis fatty acids, trans fatty acids, glycerides, monoglycerides, diglycerides, triglycerides, hormones, steroids (e.g., cholesterol, bile acids), vitamins (e.g., vitamin E), phospholipids, sphingolipids, and lipoproteins.

Those skilled in the art will recognize that this is an illustrative, not comprehensive, list of therapeutic, diagnostic, imaging and/or prophylactic agents that can be delivered in combination with the immunogenic nanoparticles described herein. Any therapeutic, diagnostic, and/or prophylactic agent may be administered with the immunogenic nanoparticles described herein.

### Kits

In various embodiments kits are provided for inducing an immune response directed against a virus are provided. In various embodiments the kits comprise a container containing one or more of the immunogenic nanoparticles described herein.

In addition, the kits optionally include labeling and/or instructional materials providing directions (e.g., protocols) for the use of the immunogenic nanoparticles described herein, e.g., alone or in with, e.g., various immune stimulants, for the treatment or prophylaxis of various viral diseases.

While the instructional materials in the various kits typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Preparation of Immunogenic Nanoparticles

The inventors have already accomplished synthesis of a PLGA nanocarrier platform that has been used successfully for an immunogenic immunotherapy (to allergens and autoimmune disease) approach in mice, as well as developing a nanocarrier for improved delivery of a potent immune adjuvant (STING agonist) to antigen-presenting cells (APC) for robust induction of Type I interferon generation. The STING nanoparticles are intended to reprogram immunogenic antigen-presenting cells (APCs) in the liver. The STING nanoparticle was constructed as a PLGA (or other biocompatible) nanocarrier that encapsulates a cyclic dinucleotide STING (“stimulator of interferon genes”) agonist for use in cancer immunotherapy. STING acts as a key intracellular target for double-stranded DNA that is released in macrophages and APCs by infectious agents, and enables the APCs to generate an immune response by activating a signal transduction pathway that results in Type I interferon release. We have confirmed that intracellular delivery of a cyclic dinucleotide STING agonist by an encapsulating PLGA nanoparticle is capable of inducing interferon-0 production in a myeloid cell line.

In this example we expand this concept by providing a nanoparticle that co-encapsulates and delivers a STING agonist (and/or other adjuvants) together with one or more viral antigens (e.g., viral proteins and/or protein fragments) for stimulating and in vivo immune response that, in certain embodiments protects against the virus.

Here, we describe the design of a 1 generation PLGA carrier to co-encapsulate COVID-19 antigens (e.g., the S-spike protein) as well as a STING agonist in a nanoparticle that can deliver both components to the immune system in mice and ultimately in humans.

One illustrative synthesis procedure incorporates the SARS-CoV-2 S protein and/or related T-cell epitopes into the carrier for delivery of a viral antigen plus a STING agonist acting as robust adjuvant. In certain embodiments PLGA nanoparticles are synthesized in the ˜500-800 nm size range, using a water/oil/water (w/o/w) method. This involves the preparation of a primary emulsion in which the S protein and/or protein fragment(s) (e.g., representative epitope peptides) are sonicated in the presence of PLGA and the STING agonist suspended in a DCM solvent. This is followed by sonicating the primary emulsion in a sodium cholate solution (secondary emulsion) and then solvent removal.

For experimental purposes, we can endow the particles with a fluorescent tag (e.g., Dylight680) S protein or including a labeled STING agonist, allowing us to determine the particle and/or active pharmaceutical ingredient (API) distribution after local or systemic injection. The synthesized particles are then subjected to comprehensive physicochemical characterization (e.g., size, zeta potential, TEM analysis, cargo loading capacity, release characteristics) followed by in vivo testing. For efficacy analysis in mice, the animals are immunized by intramuscular injection of the NPs on day 0, 14, 28. Responses are compared to injecting antigen alone, and/or other conventional adjuvants, such as alum. On day 35 after the first immunization, sera can be collected for antibody analysis, and mice are sacrificed to collect spleens and lymph nodes to determine the induction of cellular immune responses. In certain embodiments the nanoparticles range in size from about 50 nm up to about 3 μm. In certain embodiments 500-800 nm nanoparticles are used, which is one desirable size range for phagocytic uptake by macrophages and dendritic cells. These APCs migrate to the regional lymph nodes and spleen, where the antigens are presented to cognate immune cells to simulate antigen-specific cellular and humoral immune responses.

The invention can also be practiced by systemic (e.g., intravenous) administration, where a 500-800 particle size range is advantageous for biodistribution to the reticuloendothelial system and the spleen (where the particles are phagocytosed by APCs capable of initiating immunity against infectious agents).

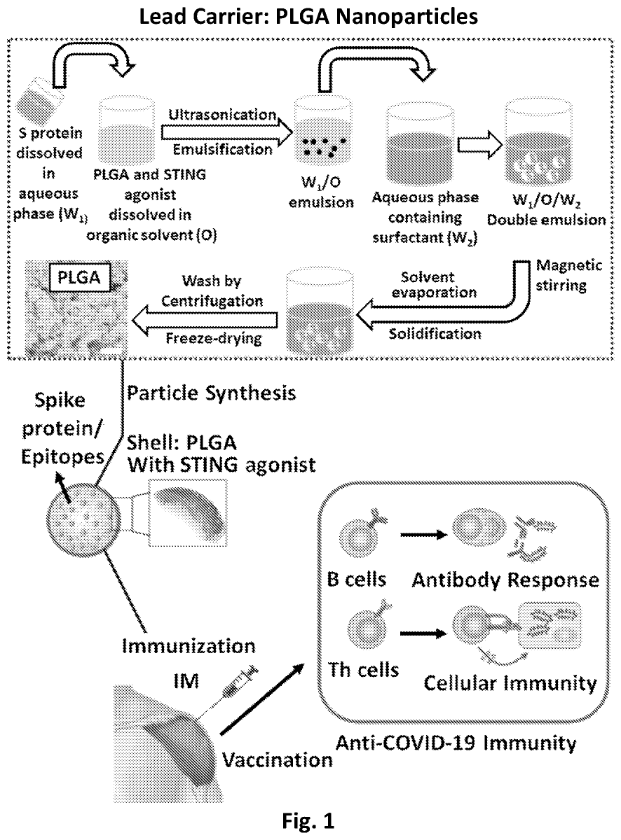

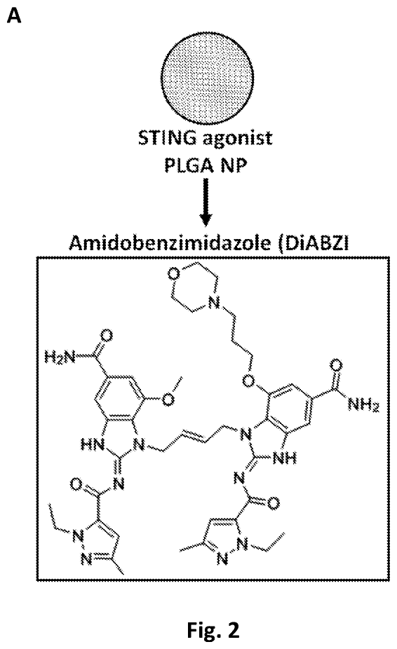

FIG. 1 illustrates development of the 1st generation of PLGA nanoparticles for immunotherapy against COVID-2019. PLGA nanoparticles, in the 500-800 nm size range are synthesized to encapsulate both the viral spike (S) protein and the STING agonist, using a double emulsion (w/o/w) method, followed by solvent removal. These particles are intramuscularly administered to the mice to test their in vivo efficacy for generating humoral and cellular immune responses. By using the routinely “prime-boost-boost” immunization scheme, the sera and splenocytes are collected to perform the antibody analysis and to assess the generation of epitope-specific cellular immune responses. The main goal for the nanoparticle development is to provide a nano-enabled antigen delivery and adjuvant platform for activating APC to prime B cells to produce neutralizing antibodies, as well as promoting TH1 differentiation to generate a protective cytotoxic T-cell response.

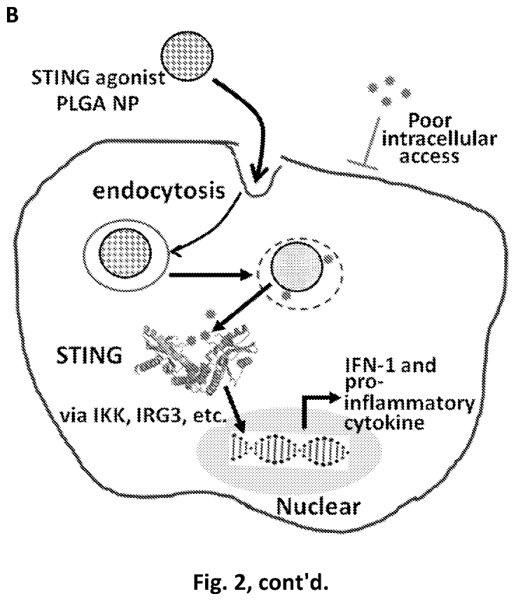

We have chosen amidobenzimidazole (diABZI), one preferred STING (stimulator of interferon genes) agonist for the 1^{st} generation carrier. STING can be activated by this agonist to initiate a signaling cascade that culminates in the expression of interferon-β (IFN-β) as well as other nuclear factor-κB (NF-κB) dependent inflammatory cytokines. Type I interferons provide a significant boost to the generation of protective immune responses. The use of a nanoparticle to deliver the STING agonist is ideal because the receptor is intracellularly located on the endoplasmic reticulum, which precludes the hydrophilic, cell wall impenetrable diABZI agonist from reaching its receptor (FIG. 2). We have used PLGA nanoparticles to successfully deliver diABZI to THP-1 cells, demonstrating effective induction of IFN-β production. Based on these results, the encapsulation of diABZI in PLGA nanoparticles will be useful for getting the adjuvant across the APC surface membrane at sites of regional antigen capture after local injection.

FIG. 2 illustrates that PLGA (or other biocompatible polymers) nanoparticles can facilitate robust STING activation. In particular, we have demonstrated that PLGA nanoparticles can improve uptake and enhance STING activation. PLGA nanoparticles incorporating the STING agonist (diABZI) could promote endocytic uptake by APCs, leading to downstream triggering of IFN-I and inflammatory cytokine production. This leads to the generation of robust cellular and humoral immune responses to the virus.

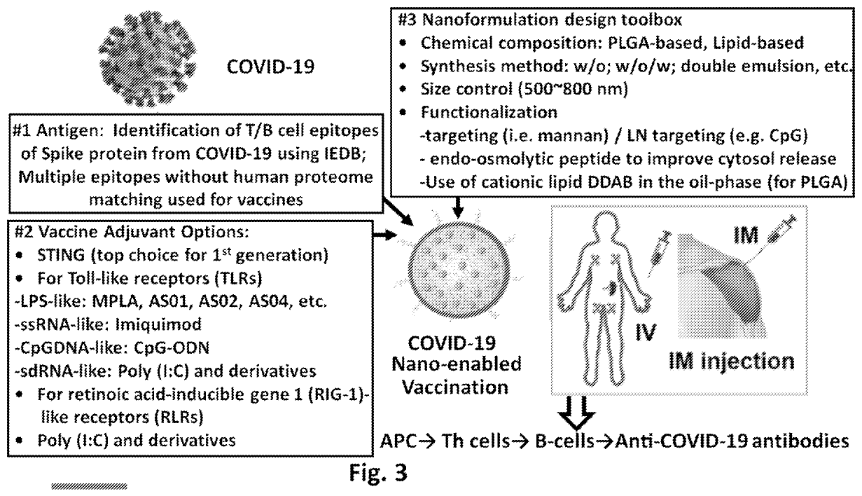

The immunogenic nanoparticles described above serve as a launch pad for a rapid carrier design, that can be further adapted for a nano-enabled vaccination strategy to COVID-19 and other viral infections (FIG. 3). The initial strategy of using whole recombinant SARS-CoV-2 spike protein can be replaced by using, for example bioinformatically screened peptide antigens (see, e.g., Baruah & Bose (2020) J. Med. Virol. 92. doi.org/10.1002/jmv.25698; Grifoni et al. (2020) Cell Host & Microbe, doi.org/10.1016/j. chom.2020.03.002). These peptide antigens elicit both T- or B-cell responses that can be combined in co-encapsulating PLGA particles, with a view to provide complementary multi-epitope cellular and humoral immunity. In addition to the use of the spike protein, it is known that coronavirus nucleocapsid, matrix and non-structural (NS) proteins (N terminal RdRp conserved across all animal and human coronaviruses) can play a role in the generation of immune responses. While neutralizing antibodies against the spike protein is of particular significance, studies using sera from the SARS outbreak revealed that the presence of IgG2a antibodies to the nucleocapsid is also important and that the humoral immune response is boosted by the presence of cytotoxic T-cells that respond against the same antigens (dos Santos et al. (2005) Virology, 331: 128-35).

The initial lead carrier is premised on the use of a STING agonist to deliver a robust adjuvant effect that strengthens the viral immune response (Luo et al. (2019) Frontiers Immunol. 10. doi.org/10.3389/fimmu.2019.02274). It is also important to consider that STING agonists are intended to reach an intracellular target in the antigen-processing APC, requiring that the agonists and co-encapsulated antigens be released from the endocytic compartment to the cytosol for further processing. In order to improve cytosolic release of the cargo from the phagosomal and endosomal compartments, it is possible to use endo-osmolytic peptides (e.g., MPG, Pep-1 and PPTG1) that destabilizes the endolysosomal membrane. Other STING agonists that can be used for encapsulation include: c-Di-AMP sodium salt, c-Di-GMP sodium salt, 2′,3′-cGAMP sodium salt, 3′,3′-cGAMP sodium salt, CMA, DMXAA STING agonist MK-1454, small molecule STING agonist such as CRD5500, etc. Please notice that certain STING agonists, such as diABZI, may not require the use of endo-osmolytic peptide due to its intrinsic properties of gaining cytosol access.

In certain embodiments the nanoparticle itself may be entirely composed of a PLGA polymer or may also contain lipid components, including the immune-activating danger signal didodecyldimethylammonium bromide (DDAB) ([Liu et al. (2018) Mol. Pharmaceutics, 15(11) 5227-5235; and the like). In certain embodiments, as described herein, the nanoparticle material may further comprise a cationic polymer and/or cationic lipid to facilitate incorporation of a nucleic acid and/or to provide a cellular “danger signal”, and/or the nanoparticle can be coated with a cationic material to facilitate attachment of a nucleic acid.

In addition to intramuscular administration, it is also possible to use intradermal, parenteral, IV, or nasal administration of PLGA nanoparticles to elicit vaccination responses. While it is likely that these responses are best promoted by particles in the 500-800 nm size range (for the reasons stated above), it is easy to adapt particle size for different use formats. It is also possible to envisage decorating the particle surface with targeting ligands such as mannan oligosaccharide groups (see, e.g., Apostolopoulous et al. (2013) J. Drug Delivery, doi.org/10.1155/2013/869718). We can also add surface ligands to target the particles to local lymph nodes from the intramuscular or regional injection sites. Examples include, but are not limited to CpG (see, e.g., Thomas, et al. (2014) Biomaterials, 35(2): 814-824) and the novel amph-CpG family (see, e.g., Irvine et al. (2014) Nature, 507: 519-522).

FIG. 3 schematically illustrates the overall design toolbox to design a nano-enabled COVID-19 (or other viruses) vaccination.

### Example 2

### PLGA Nanoparticles Induce Antigen-Specific Antibodies in an Animal Prophylactic Vaccine Model

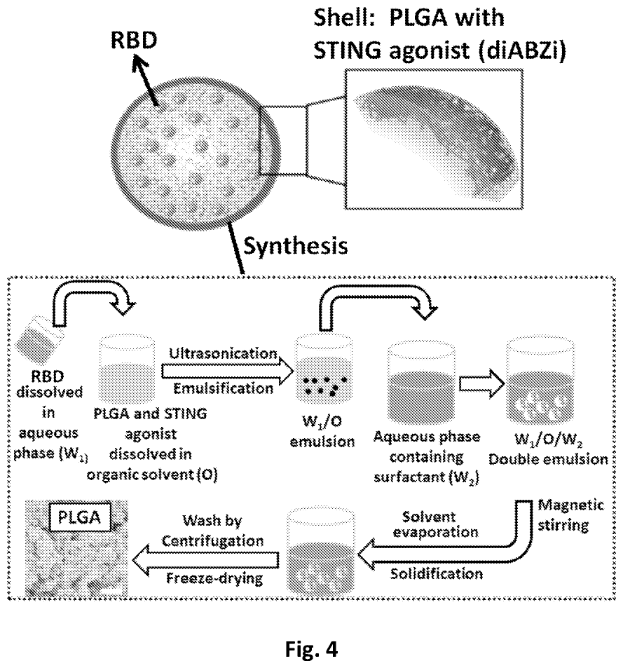

Due to the lack of preexisting human immunity, the SARS-CoV-2 virus's rapid spread, and COVID-19's relatively high hospitalization and fatality rate, developed a nanoparticle-based vaccine for the delivery of COVID-19 antigens and adjuvants to generate potent immune response against the virus. The basic platform we applied is comprised of a poly (lactic-co-glycolic acid) (PLGA) copolymer, approved by the FDA for various therapeutic applications by FDA because of its biodegradability and biocompatibility. The PLGA nanoparticles incorporating RBD and a STING agonist were synthesized by double emulsion method (w/o/w) combined with solvent removal technique (FIG. 4). The particles NP/sting-RBD were then characterized with the uniform size of ˜240 nm and PDI of 0.145. The surface charge remained negative for about −50 mV (Table 17).

TABLE 17
Nanoparticle formulations for hydrodynamic size, zeta potential, antigen
content, and sting agonist content. The nanoparticles were characterized
using dynamic light scattering to determine particle size and surface
charge. The microBCA assay was used to detect antigen loading capacity
and the diABZi content was determined by HPLC.

| | | | | Antigen | |
|---|---|---|---|---|---|
| | Hydro- | | | Loading | diABZi |
| | dynamic | | Zeta | capacity | content |
| Nano- | size | | potential | (μg/mg | (μg/mg |
| particle | (nm) | PDI | (mV) | NPs) | NPs) |
| Bare NP | 225.7 ± 6.94 | 0.130 | −61.36 ± 1.15 | NA | Approx. |
| | | | | | ~107 |
| NP/sting- | 241.9 ± 7.34 | 0.145 | −50.92 ± 0.39 | 35.73 ± 4.61 | Approx. |
| RBD | | | | | ~107 |

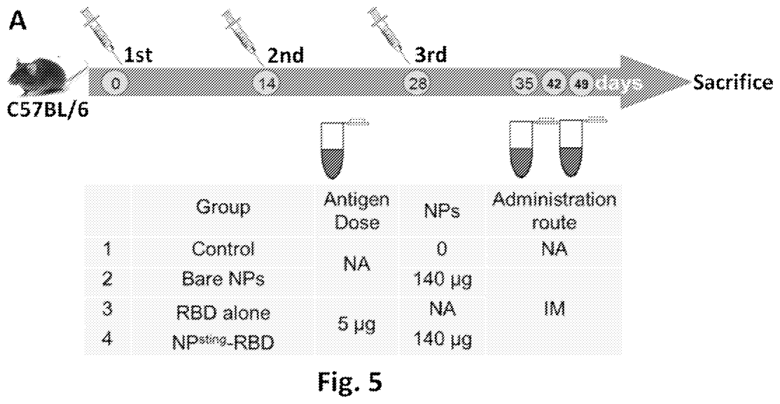

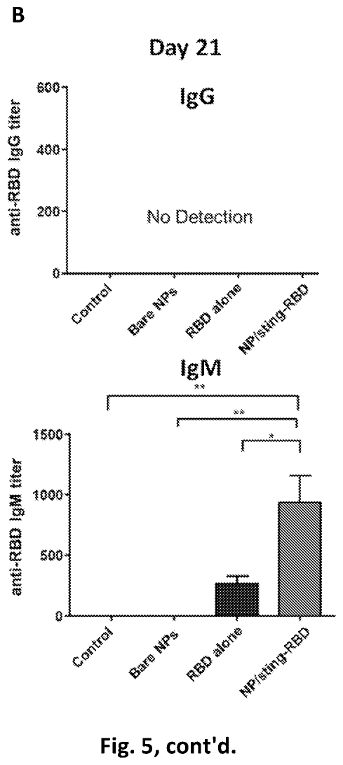

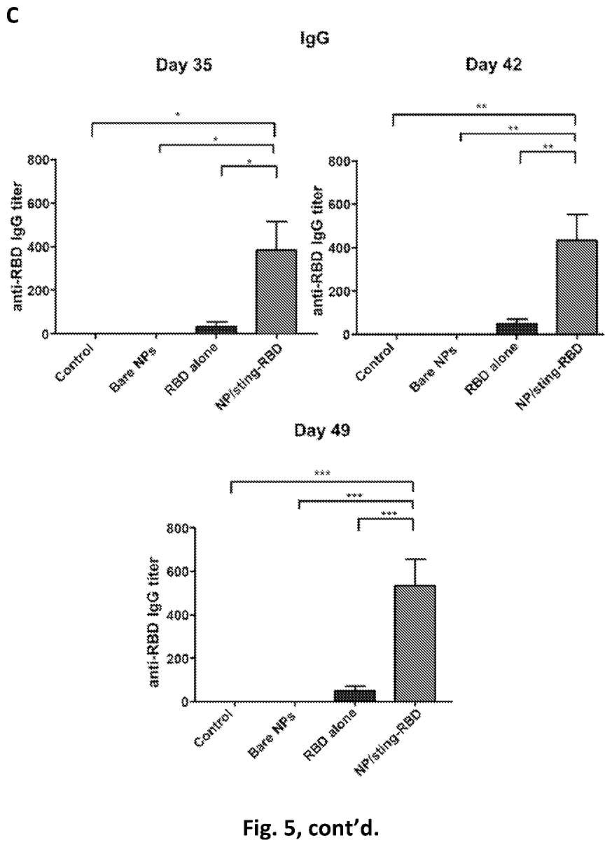

The incorporation capacity was also determined with the antigen loading amount of ˜35 μg and agonist (diABZi) 107 μg per mg particle, respectively. The bare nanoparticles without antigen and agonist were also fabricated with similar size (˜226 nm) and surface charge (−61 mV) and used as control nanoparticle (Table 17). The particle-based vaccine was intramuscularly administered into C57BL/6 mice by using the routinely “prime-boost-boost” immunization scheme to test the in vivo efficacy (FIG. 5). Briefly, mice (6-8 weeks-of-age, n=6, female) were i.m. administered with a 100 μL (50 μL per hind leg) suspension of saline, blank particles, RBD alone and NP/sting-RBD. Dose of NPs used in immunization were 140 μg NPs per mouse per injection and the dose of RBD were 5 μg per mouse. The mice were injected i.m on day 0, 14, and 28. On day 21, 35, 42 and 49 after the first immunization, sera were collected from the venous plexus of the mice's posterior orbits for antibody analysis. Mice were sacrifice on day 49. As expected, NP/sting-RBD enhanced the production of RBD-specific IgM on day 21 (IgM antibody titer ˜900), which showed the robust initiation of humoral immune response. In addition, NP/sting-RBD-treated mice exhibited strong anti-RBD IgG responses (IgG antibody titer ˜380) starting from day 35 after vaccination, while injection with RBD alone induced only a weak immunogenic response (IgG antibody titer ˜50). The level of RBD IgG responses sustained from day 35 to day 42 (IgG antibody titer ˜430) and increased at day 49 with IgG antibody titer ˜530 (FIG. 5), which were significantly higher than RBD alone (p<0.05). These findings demonstrate that the designed PLGA NP-based vaccine platform is capable of inducing antigen-specific immune response, which may be useful to combat COVID-19.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### SEQUENCE LISTING

```
SARS-CoV-2 Spike Protein
P59594-1 [UniParc]FASTA
Sequence ID No: 1
                10 20 30 40
MFIFLLFLTL TSGSDLDRCT TFDDVQAPNY TQHTSSMRGV
                50 60 70 80
YYPDEIFRSD TLYLTQDLFL PFYSNVTGFH TINHTFGNPV
                90 100 110 120
IPFKDGIYFA ATEKSNVVRG WVFGSTMNNK SQSVIIINNS
              130 140 150 160
TNVVIRACNF ELCDNPFFAV SKPMGTQTHT MIFDNAFNCT
              170 180 190 200
FEYISDAFSL DVSEKSGNFK HLREFVEKNK DGFLYVYKGY
              210 220 230 240
QPIDVVRDLP SGFNTLKPIF KLPLGINITN FRAILTAFSP
              250 260 270 280
AQDIWGTSAA AYFVGYLKPT TFMLKYDENG TITDAVDCSQ
              290 300 310 320
NPLAELKCSV KSFEIDKGIY QTSNFRVVPS GDVVRFPNIT
              330 340 350 360
NLCPFGEVFN ATKFPSVYAW ERKKISNCVA DYSVLYNSTF
              370 380 390 400
FSTFKCYGVS ATKLNDLCFS NVYADSFVVK GDDVRQIAPG
              410 420 430 440
QTGVIADYNY KLPDDFMGCV LAWNTRNIDA TSTGNYNYKY
              450 460 470 480
RYLRHGKLRP FERDISNVPF SPDGKPCTPP ALNCYWPLND
              490 500 510 520
YGFYTTTGIG YQPYRVVVLS FELLNAPATV CGPKLSTDLI
              530 540 550 560
KNQCVNFNFN GLTGTGVLTP SSKRFQPFQQ FGRDVSDFTD
              570 580 590 600
SVRDPKTSEI LDISPCSFGG VSVITPGTNA SSEVAVLYQD
              610 620 630 640
VNCTDVSTAI HADQLTPAWR IYSTGNNVFQ TQAGCLIGAE
              650 660 670 680
HVDTSYECDI PIGAGICASY HTVSLLRSTS QKSIVAYTMS
              690 700 710 720
LGADSSIAYS NNTIAIPTNF SISITTEVMP VSMAKTSVDC
              730 740 750 760
NMYICGDSTE CANLLLQYGS FCTQLNRALS GIAAEQDRNT
              770 780 790 800
REVFAQVKQM YKTPTLKYFG GFNFSQILPD PLKPTKRSFI
              810 820 830 840
EDLLFNKVTL ADAGFMKQYG ECLGDINARD LICAQKFNGL
              850 860 870 880
TVLPPLLTDD MIAAYTAALV SGTATAGWTF GAGAALQIPF
              890 900 910 920
AMQMAYRFNG IGVTQNVLYE NQKQIANQFN KAISQIQESL
              930 940 950 960
TTTSTALGKL QDVVNQNAQA LNTLVKQLSS NFGAISSVLN
              970 980 990 1000
DILSRLDKVE AEVQIDRLIT GRLQSLQTYV TQQLIRAAEI
            1010 1020 1030 1040
RASANLAATK MSECVLGQSK RVDFCGKGYH LMSFPQAAPH
            1050 1060 1070 1080
GVVFLHVTYV PSQERNFTTA PAICHEGKAY FPREGVFVFN
            1090 1100 1110 1120
GTSWFITQRN FFSPQIITTD NTFVSGNCDV VIGIINNTVY
            1130 1140 1150 1160
DPLQPELDSF KEELDKYFKN HTSPDVDLGD ISGINASVVN
            1170 1180 1190 1200
IQKEIDRLNE VAKNLNESLI DLQELGKYEQ YIKWPWYVWL
            1210 1220 1230 1240
GFIAGLIAIV MVTILLCCMT SCCSCLKGAC SCGSCCKFDE
            1250
DDSEPVLKGV KLHYT
```

What is claimed is:

1. An immunogenic nanoparticle comprising:
a nanoparticle comprising a biocompatible polymer;
one or more viral proteins or fragment(s) thereof encapsulated within or attached to said nanoparticle where the viral protein(s) or fragment(s) thereof comprises an antigen to which an immune response is to be induced by administration of said immunogenic nanoparticle to a mammal; and
an adjuvant.

2. The immunogenic nanoparticle of claim 1, wherein said one or more proteins or fragments are conjugated to, or co-encapsulated with, said adjuvant.

3. An immunogenic nanoparticle comprising:
a nanoparticle comprising a biocompatible polymer; and
a nucleic encapsulated within or attached to said nanoparticle where said nucleic acid encodes one or more viral protein(s) or fragment(s) thereof that comprise one or more antigen(s) to which an immune response is to be induced by administration of said immunogenic nanoparticle to a mammal.

4. The immunogenic nanoparticle of claim 3, wherein said nanoparticle further comprises an adjuvant.

5. The immunogenic nanoparticle of claim 4, wherein said adjuvant is conjugated to said nucleic acid.

6. The immunogenic particle according to any one of claims 3-5, wherein said nucleic acid comprises an mRNA.

7. The immunogenic nanoparticle of claim 6, wherein said nucleic acid comprises an mRNA comprising a cap, 5′ and 3′ untranslated regions (UTRs), an open-reading frame (ORF) that encodes said one or more viral protein(s) or fragment(s) thereof that comprise one or more antigen(s) to which an immune response is to be induced, and a 3′ poly(A) tail.

8. The immunogenic nanoparticle of claim 7, wherein said nucleic acid further comprises replication machinery derived from a positive-stranded mRNA virus.

9. The immunogenic nanoparticle of claim 8, wherein said nucleic acid further comprises replication machinery derived from an alphaviruses such as Sindbis or Semliki-Forest viruses.

10. The immunogenic nanoparticle according to any one of claims 1-9, wherein said biocompatible polymer comprises one or more polymers selected from the group consisting of poly(lactic-co-glycolic acid) (PLGA), poly(glycolic acid) (PGA), poly(lactic acid) (PLA), poly(caprolactone) (PCL), poly(butylene succinate), poly(trimethylene carbonate), poly(p-dioxanone), Poly(butylene terephthalate), poly(ester amide) (HYBRANE®), polyurethane, poly[(carboxyphenoxy) propane-sebacic acid], poly[bis(hydroxyethyl) terephthalate-ethyl orthophosphorylate/terephthaloyl chloride], poly(β-hydroxyalkanoate), poly(hydroxybutyrate), poly(hydroxybutyrate-co-hydroxyvalerate), Poly-L-lysine (PLL), poly-ethylenimine (PEI), poly[(2-dimethylamino)ethyl methacrylate (pDMAEMA), polyamidoamine (PAMAM) dendrimers, biodegradable poly(β-amino ester) (PBAE) polymer, and poly(amino-co-ester) (PACE) polymers.

11. The immunogenic nanoparticle according to any one of claims 3-9, wherein said biocompatible polymer comprises a cationic polymer.

12. The immunogenic nanoparticle of claim 11, wherein said biocompatible polymer comprises a cationic polymer selected from the group consisting of Poly-L-lysine (PLL), poly-ethylenimine (PEI), poly[(2-dimethylamino)ethyl methacrylate (pDMAEMA), polyamidoamine (PAMAM) dendrimers, biodegradable poly(β-amino ester) (PBAE) polymer, and poly(amino-co-ester) (PACE) polymer.

13. The immunogenic nanoparticle according to any one of claims 1-9, wherein said biocompatible polymer comprises poly(lactic-co-glycolic acid) (PLGA).

14. The immunogenic nanoparticle of claim 13, wherein said PLGA comprises a lactide/glycolid molar ratio ranging from about 30:70 to about 70:30.

15. The immunogenic nanoparticle of claim 14, wherein said PLGA comprises a lactide/glycolid molar ratio of about 50:50.

16. The immunogenic nanoparticle according to any one of claims 1-15, wherein said nanoparticles are of a size effective for phagocytic uptake by macrophages and/or dendritic cells.

17. The immunogenic nanoparticle of claim 16, wherein said nanoparticle ranges in size from about 50 nm up to about 3 μm.

18. The immunogenic nanoparticle of claim 17, wherein said nanoparticle ranges in size from about 50 nm, or from about 75 nm, or from about 100 nm, or from about 125 nm, or from about 150 nm, or from about 200 nm, or from about 250 nm, or from about 300 nm, or from about 350 nm, or from about 400 nm, or from about 450 nm, or from about 500 nm up to about 3 μm, or up to about 2.75 μm, or up to about 2.5 μm, or up to about 2.0 μm, or up to about 1.75 μm, or up to about 1.50 μm, or up to about 1.25 μm, or up to about 1 μm, or up to about 900 nm, or up to about 800 nm.

19. The immunogenic nanoparticle of claim 18, wherein said nanoparticle ranges in size from 500 to 800 nm.

20. The immunogenic nanoparticle according to any one of claims 1-19, wherein the material forming said nanoparticle consists entirely of said biocompatible polymer and said viral proteins or fragment(s) thereof and/or said nucleic acid.

21. The immunogenic nanoparticle according to any one of claims 1-19, wherein the material forming said nanoparticle consists of a biocompatible PLGA polymer that is coated with chitosan (a linear polysaccharide, composed of randomly distributed D-glucosamine plus N-acetyl-D-glucosamine), to provide a particle surface that allows nucleic acid binding.

22. The immunogenic nanoparticle according to any one of claims 1-19, wherein said nanoparticle additionally comprises a lipid.

23. The immunogenic nanoparticle of claim 22, wherein said lipid is a lipid capable of activating a danger signal and/or binding a nucleic acid.

24. The immunogenic nanoparticle of claim 23, wherein said lipid comprises a lipid selected from the group consisting of didodecyldimethylammonium bromide (DDAB), and 1,2-dioleoyloxy-3-trimethylammonium propane chloride (DOTAP), DOTMA (N-[1-(2,3-dioleyloxy)propyl]-N,N,N-trimethylammonium chloride), 1,2-dilinoleyloxy-3-dimethylaminopropane (DLinDMA), dilinoleylmethyl-4 dimethyl aminobutyrate (DLin-MC3-DMA), C12-200 (Lipid 5), 3-(dimethylamino) propyl(12Z,15Z)-3-[(9Z,12Z)-octadeca-9,12-dien-1-yl]henicosa-12,15-dienoate (DMAP-BLP), and (2Z)-non-2-en-1-yl 10-[(Z)-(1-methylpiperidin-4 yl)carbonyloxy]nonadecanoate (L101).

25. The immunogenic nanoparticle according to any one of claims 22-24, wherein said lipid comprises up to 25%, (molar percentage) of said nanoparticle.

26. The immunogenic nanoparticle of claim 25, wherein said lipid comprises about 1%, or about 2%, or about 3%, or about 4%, or about 5%, or about 6%, or about 7% up to about 25%, or up to about 20%, or up to about 15%, or up to about 10% (molar percentage) of said nanoparticle.

27. The immunogenic nanoparticle according to any one of claims 1-26, wherein said one or more viral proteins or fragment(s) thereof are encapsulated within said nanoparticle (e.g., incorporated in said biocompatible polymer).

28. The immunogenic nanoparticle according to any one of claims 3-27, wherein said nucleic acid(s) encoding one or more viral proteins or fragment(s) thereof are encapsulated within said nanoparticle (e.g., incorporated in said biocompatible polymer).

29. The immunogenic nanoparticle according to any one of claims 1-28, wherein said one or more viral proteins or fragment(s) thereof are attached to the surface of said nanoparticle.

30. The immunogenic nanoparticle according to any one of claims 3-29, wherein said nucleic acid(s) encoding one or more viral proteins or fragment(s) thereof are attached to the surface of said nanoparticle.

31. The immunogenic nanoparticle of claim 29 or 30, wherein said one or nucleic acids or said one or more viral proteins or fragment(s) thereof are directly attached to the surface of said nanoparticle.

32. The immunogenic nanoparticle of claim 31, wherein said one or more nucleic acids or said one or more viral proteins or fragment(s) thereof are attached to the surface of said nanoparticle with a linker.

33. The immunogenic nanoparticle of claim 32, wherein said linker comprises a DSPE-PEG-maleimide where said linker is incorporated in or on said biocompatible polymer.

34. The immunogenic nanoparticle according to any one of claims 1-33, wherein said one or more viral proteins or fragments thereof are derived from a virus selected from the group consisting of corona viruses (including, but not limited to 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), SARS-CoV-2 (the novel coronavirus that causes coronavirus disease 2019, or COVID-19), and the like), human influenza viruses Types A and B, human Rhinovirus, respiratory syncytial virus (e.g., sin-SISH-uhl), parainfluenza viruses (HPIVs), measles virus, rubella virus, chickenpox/shingles virus, smallpox, chikungunya virus, hepatitis viruses, herpes viruses, human papilloma viruses (HPVs), polio virus, Norovirus, Rotavirus, Marburg virus, Ebola virus, Rabies, Smallpox, HIV, Hantavirus, Dengue, Molluscum contagiosum, Herpes simplex virus-1 (HSV-1), and Varicella-zoster virus (VZV).

35. The immunogenic nanoparticle according to any one of claims 1-19, wherein said one or more viral proteins or fragments thereof are derived from a virus weaponized or studied as an agent for biological warfare.

36. The immunogenic nanoparticle of claim 35, wherein said virus is selected from the group consisting of the Bunyaviridae (especially Rift Valley fever virus), Ebolavirus, many of the Flaviviridae (especially Japanese encephalitis virus), Machupo virus, Marburg virus, Variola virus, yellow fever virus, viral encephalitis alphaviruses (e.g., venezuelan equine encephalitis, eastern equine encephalitis, western equine encephalitis), hantaviruses, Nipah virus, Tickborne encephalitis viruses, tickborne hemorrhagic fever viruses, and Yellow fever.

37. The immunogenic nanoparticle of claim 34, wherein said corona virus comprise a member of the Betacoronavirus Genus.

38. The immunogenic nanoparticle of claim 37, wherein said corona virus comprises a corona virus that produces severe acute respiratory syndrome (SARS).

39. The immunogenic nanoparticle of claim 38, said corona virus comprises a corona virus selected from the group consisting of that SARS-CoV-2 (2019-nCoV), SARS-CoV-2, and MERS-CoV.

40. The immunogenic nanoparticle of claim 39, wherein said corona virus is SARS-CoV-2 (2019-nCoV).

41. The immunogenic nanoparticle according to any one of claims 34-40, wherein said one or more viral proteins or fragments thereof comprise a corona virus protein or fragment thereof.

42. The immunogenic nanoparticle of claim 41, wherein said one or more viral proteins or fragments thereof comprise a corona virus protein or fragment thereof from a coronavirus that is a member of the Betacoronavirus genus.

43. The immunogenic nanoparticle according to any one of claims 41-42, wherein said one or more viral proteins or fragments thereof comprise an N-protein or fragment thereof, an M-protein or fragment thereof, and/or an S-protein or fragment thereof.

44. The immunogenic nanoparticle of claim 43, wherein said viral protein(s) or fragment(s) thereof comprise a full-length S-protein.

45. The immunogenic nanoparticle of claim 43, wherein said viral protein(s) or fragment(s) thereof comprise a full length S1 subunit of an S-protein.

46. The immunogenic nanoparticle of claim 43, wherein said viral protein(s) or fragment(s) thereof comprise a full length S2 subunit of an S-protein.

47. The immunogenic nanoparticle of claim 43, wherein said viral proteins or fragments thereof comprises an S protein or S protein fragment comprising a mutation selected from the group consisting of D614G, L452R, E484Q, and E484K.

48. The immunogenic nanoparticle of claim 47, wherein said viral proteins or fragments thereof comprises an S protein or S protein fragment comprising a D614G mutation.

49. The immunogenic nanoparticle of according to any one of claims 47-48, wherein said viral proteins or fragments thereof comprises an S protein or S protein fragment comprising an L452R mutation.

50. The immunogenic nanoparticle of according to any one of claims 47-49, wherein said viral proteins or fragments thereof comprises an S protein or S protein fragment comprising an E484Q mutation.

51. The immunogenic nanoparticle of according to any one of claims 47-50, wherein said viral proteins or fragments thereof comprises an S protein or S protein fragment comprising an E484K mutation.

52. The immunogenic nanoparticle according to any one of claims 43-51, wherein said viral protein(s) or fragment(s) thereof comprise a full-length N-protein.

53. The immunogenic nanoparticle according to any one of claims 43-51, wherein said viral protein(s) or fragment(s) thereof comprise a fragment of an N-protein.

54. The immunogenic nanoparticle according to any one of claims 43-53, wherein said viral protein(s) or fragment(s) thereof comprise a T cell epitope from a SARS-CoV-2 S-protein.

55. The immunogenic nanoparticle of claim 54, wherein said viral protein(s) or fragment(s) thereof comprise a T cell epitope from a SARS-CoV-2 S-protein shown in Table 1, or an immunogenic fragment thereof.

56. The immunogenic nanoparticle of claim 54, wherein said viral protein(s) or fragment(s) thereof comprise a T cell epitope of the SARS-CoV-2 S-protein comprising or consisting of the sequence PAQEKNFTTAPAICH (SEQ ID NO:1), or an immunogenic fragment thereof.

57. The immunogenic nanoparticle of claim 54, wherein said viral protein(s) or fragment(s) thereof comprise a T cell epitope of the SARS-CoV-2 S-protein comprising or consisting of the sequence GYHLMSFPQSAPHGV (SEQ ID NO:2), or an immunogenic fragment thereof.

58. The immunogenic nanoparticle of claim 54, wherein said viral protein(s) or fragment(s) thereof comprise a T cell epitope of the SARS-CoV-2 S-protein comprising or consisting of the sequence ARSVASQSIIAYTMS (SEQ ID NO:3), or an immunogenic fragment thereof.

59. The immunogenic nanoparticle of claim 54, wherein said viral protein(s) or fragment(s) thereof comprise a T cell epitope of the SARS-CoV-2 S-protein comprising or consisting of the sequence IDGYFKIYSKHTPIN (SEQ ID NO:4), or an immunogenic fragment thereof.

60. The immunogenic nanoparticle of claim 54, wherein said viral protein(s) or fragment(s) thereof comprise a T cell epitope of the SARS-CoV-2 S-protein comprising or consisting of the sequence QMAYRFNGIGVTQNV (SEQ ID NO:5), or an immunogenic fragment thereof.

61. The immunogenic nanoparticle according to any one of claims 43-60, wherein said viral protein(s) or fragment(s) thereof comprise one or more T cell epitopes from a SARS-CoV-2 or SARS-COV N-protein shown in Table 2 (SEQ ID NOs:6-21), or an immunogenic fragment thereof.

62. The immunogenic nanoparticle according to any one of claims 43-61, wherein said viral protein(s) or fragment(s) thereof comprise one or more T cell epitopes from a SARS-CoV-2 or SARS-CoV S-protein shown in Table 2 (SEQ ID NOs:22-32), or an immunogenic fragment thereof.

63. The immunogenic nanoparticle according to any one of claims 43-62, wherein said viral protein(s) or fragment(s) thereof comprise one or more T cell epitopes from a SARS-CoV-2 or SARS-CoV N-protein and/or S-protein shown in Table 3 (SEQ ID NOs:33-208), or an immunogenic fragment thereof.

64. The immunogenic nanoparticle according to any one of claims 43-63, wherein said viral protein(s) or fragment(s) thereof comprise an epitope located in the SARS-CoV receptor-binding motif.

65. The immunogenic nanoparticle of claim 64, wherein said viral protein(s) or fragment(s) thereof comprise an epitope comprising or consisting of the a sequence selected from the group consisting of QPYRVVVLSF (SEQ ID NO:643), GYQPYRVVVL (SEQ ID NO:59), and PYRVVVLSF (SEQ ID NO:60), or an immunogenic fragment thereof.

66. The immunogenic nanoparticle according to any one of claims 43-65, wherein said viral protein(s) or fragment(s) thereof comprise a SARS-CoV-2 and/or SARS-CoV B cell epitope.

67. The immunogenic nanoparticle of claim 66, wherein said viral protein(s) or fragment(s) thereof comprise or consist of a B cell S-protein epitope shown in Table 4 (SEQ ID NOs: 210-232).

68. The immunogenic nanoparticle according to any one of claims 66-67, wherein said viral protein(s) or fragment(s) thereof comprise or consist of a B cell N-protein epitope shown in Table 4 (SEQ ID NOs: 233-254).

69. The immunogenic nanoparticle according to any one of claims 41-68, wherein said viral protein(s) or fragments thereof comprise or consist of a SARS-CoV B cell epitope shown in Table 5, or an immunogenic fragment thereof.

70. The immunogenic nanoparticle according to any one of claims 41-69, wherein said viral protein(s) or fragments thereof comprise or consist of a SARS-CoV T cell epitope shown in Table 6, or an immunogenic fragment thereof.

71. The immunogenic nanoparticle according to any one of claims 41-70, wherein said viral protein(s) or fragments thereof comprise or consist of a SARS-CoV-2 B cell epitope shown in Table 5, or an immunogenic fragment thereof.

72. The immunogenic nanoparticle according to any one of claims 41-71, wherein said viral protein(s) or fragments thereof comprise or consist of a SARS-CoV-2 T cell epitope shown in Table 6, or an immunogenic fragment thereof.

73. The immunogenic nanoparticle according to any one of claims 34-72, wherein said one or more viral proteins or fragments thereof comprise or consist of an amino acid sequence shown in one or more of Tables 10-12.

74. The immunogenic nanoparticle according to any one of claims 34-73, wherein said one or more viral proteins or fragments thereof comprise or consist of an amino acid sequence shown in shown in Table 7 and/or in Table 8.

75. The immunogenic nanoparticle of claim 74, wherein said one or more viral proteins or fragments thereof comprise or consist of an amino and amino acid sequence shown in Table 7 and an amino acid sequence shown in Table 8.

76. The immunogenic nanoparticle according to any one of claims 34-75, wherein said one or more viral proteins or fragments thereof comprise or consist of an amino acid sequence shown in shown in Table 9.

77. The immunogenic nanoparticle according to any one of claims 1-79, wherein said adjuvant comprises an adjuvant that elicits a TH1-biased immune response.

78. The immunogenic nanoparticle of claim 77, wherein said adjuvant is selected from the group consisting of a combined aluminum salt and TLR4 agonist, rOv-ASP-1 (recombinant Onchocerca volvulus activation associated protein-1, IC31® (a two-component adjuvant consisting of the artificial antimicrobial cationic peptide KLK acting as a vehicle and the TLR9-stimulatory oligodeoxynucleotide ODN1, SPO1, CPG oligonucleotide, alum-TLR7 agonist based on a TLR7 agonist (SMIP7.10), OprI lipoprotein of Pseudomonas aeruginosa, cathelicidin-derived antimicrobial peptides, delta inulin (β-D-[2-1]poly(fructo-furanosyl)α-D-glucose), β-defensin, and a STING agonist.

79. The immunogenic nanoparticle of claim 78, wherein said adjuvant comprises one or more STING agonists.

80. The immunogenic nanoparticle of claim 79, wherein said adjuvant comprises one or more STING agonists selected from the group consisting of amidobenzimidazole (diABZI), 3′,5′-Cyclic diadenylic acid sodium salt (c-DI-AMP sodium salt), 3′,5′-Cyclic diguanylic acid sodium salt (c-Di-GMP sodium salt), 2′,3′-Cyclic guanosine monophosphate-adenosine monophosphate sodium salt (2′,3′-cGAMP), 3′,3′-Cyclic guanosine monophosphate-adenosine monophosphate sodium salt (3′,3′-cGAMP), 5,6-Dimethyl-9-oxo-9H-xanthene-4-acetic acid (DMXAA), CMA, MK-1454, CRD5500, cyclic di-nucleotide compounds as described in U.S. Patent Publication No: 2020/0062798 A1, tricyclic heteroaryl compounds as described in U.S. Patent Publication No: 2020/0040009 A1, and heteroaryl amide compounds as described in U.S. Patent Publication No: 2020/0039994 A1.

81. The immunogenic nanoparticle of claim 80, wherein said adjuvant comprises amidobenzimidazole (diABZI).

82. The immunogenic nanoparticle according to any one of claims 1-81, wherein said adjuvant comprises one or more adjuvants selected from the group consisting of CpG ODNs (TLR9 agonists), imiquimod-family compounds (TLR7 agonists), lipopolysaccharide-based compounds (TLR4) LPS-like compounds, Flageline, dsRNA-like compounds (e.g., Poly (I:C) and derivatives), poly (I:C) and derivatives, MatrixM™⁹⁰, AS03, CpG 1018), and Advax.

83. The immunogenic nanoparticle according to any one of claims 1-82, wherein said nanoparticles have one or more targeting moieties attached to the surface where said targeting moieties bind to and/or facilitate uptake by antigen presenting cells (APCs).

84. The immunogenic nanoparticle of claim 83, wherein said antigen presenting cells comprise one or more cells selected from the group consisting of macrophages, dendritic cells, and B cells.

85. The immunogenic nanoparticle according to any one of claims 83-84, wherein said targeting moiety binds to a receptor on a dendritic cell.

86. The immunogenic nanoparticle of claim 85, wherein said receptor on a dendritic cell is selected from the group consisting of DEC205, MR, Dectin-1, DC-SIGN, DNGR-1, and FcγR.

87. The immunogenic nanoparticle according to any one of claims 85-86, wherein said targeting moiety is selected from the group consisting of SAG-1 (T. gondii), HIV-1 gagP24, Ova, AHc, RSV fusion protein, MUC1, MAA, hCGβ, Ova, Diphtheria toxin (CRM₁₉₇), Ag85B (Mtb), triMN-LPR, Ova, Anti-Clec9A, Ova, MUC1, MUC1-Tn, E75 (HER-2), iFT, gp120_{αgal}/p24, and α-gal.

88. The immunogenic nanoparticle according to any one of claims 83-87, wherein said targeting moiety binds to a receptor on a macrophage.

89. The immunogenic nanoparticle of claim 88, wherein said targeting moiety binds to a receptor selected from the group consisting of a sialoadhesin receptor, a folate receptor, a galactose receptor, a mannose receptor, a β-glucan receptor, a scavenger receptor, and a tuftsin receptor.

90. The immunogenic nanoparticle of claim 89, wherein said targeting moiety is selected from the group consisting of sialic acid, 9-N-(4H-thieno[3,2-c]chromene-2-carbamoyl)-Neu5Acα2-3Ga1β-4G1cNAc (TCCNeu5Ac), folic acid, methotrexate, folate, galactose residue, lactose, low density lipoprotein (LDL), ovalbumin (OVA), lactobionic acid, mannose-rich glycoconjugates, mannose, mannan, mannosylated poly(L-lysine) (MPL), zymosan and other β-glucans, glucan, poly-guanine, apoB protein fragment, and Tufsin tetrapeptide (Thr-Lys-Pro-Arg, (SEQ ID NO:644)).

91. The immunogenic nanoparticle of claim 89, wherein said targeting moiety binds to or more scavenger receptors selected from the group consisting of Stabilin 1, Stabilin 2, and mannose receptor.

92. The immunogenic nanoparticle of claim 91, wherein said targeting moiety comprises a fragment of apolipoprotein B protein effective to bind to Stabilin 1 and/or Stabilin 2.

93. The immunogenic nanoparticle of claim 92, wherein said targeting moiety fragment ranges in length from about 5, or from about 8, or from about 10 up to about 50, or up to about 40, or up to about 30, or up to about 20 amino acids.

94. The immunogenic nanoparticle of claim 93, wherein said targeting moiety comprises a fragment of the apoB protein comprising the amino acid sequence RKRGLK (SEQ ID NO:640).

95. The immunogenic nanoparticle of claim 94, wherein said first targeting moiety comprises a fragment of the apoB protein comprising the amino acid sequence RLYRKRGLK (SEQ ID NO:641).

96. The immunogenic nanoparticle of claim 94, wherein said first targeting moiety comprise or consists of the amino acid sequence CGGKLGRKYRYLR (SEQ ID NO:642).

97. The immunogenic nanoparticle of claim 89, wherein said targeting moiety binds to a mannose receptor.

98. The immunogenic nanoparticle of claim 97, wherein said targeting moiety comprises mannan.

99. The immunogenic nanoparticle of claim 98, wherein said targeting moiety comprises a mannan having a MW ranging from about 35 to about 60 kDa.

100. The immunogenic nanoparticle according to any one of claims 83-99, wherein said targeting moiety targets lymph nodes.

101. The immunogenic nanoparticle of claim 100, wherein said targeting moiety targets lymph node germinal centers.

102. The immunogenic nanoparticle of claim 101, wherein said surface of said nanoparticle is glycosylated.

103. The immunogenic nanoparticle of claim 102, wherein targeting moiety comprises a glycan-rich moiety.

104. The immunogenic nanoparticle of claim 103, wherein targeting moiety comprises the glycan-rich bacterial protein, lumazine synthase.

105. The immunogenic nanoparticle of claim 100, wherein said targeting moiety is selected from the group consisting of CpG, and a member of the amph-CpG family.

106. The immunogenic nanoparticle according to any one of claims 83-105, wherein said second binding moiety is adsorbed to said nanoparticle.

107. The immunogenic nanoparticle according to any one of claims 83-105, wherein said second binding moiety is covalently bound to said nanoparticle directly or through a linker.

108. The immunogenic nanoparticle according to any one of claims 1-107, wherein said nanoparticle contains a compound that facilitates cytosolic release from the endolysosomal compartment.

109. The immunogenic nanoparticle of claim 108, wherein said compound comprises an endo-osmolytic peptide.

110. The immunogenic nanoparticle of claim 108, wherein said compound comprises an endo-osmolytic peptide selected from the group consisting of MPG, Pep-1, and PPTG1) that destabilizes the in the endolysosomal membrane, antimicrobial peptide LL-37 with glutamic acid substituting for all basic residues, antimicrobial peptide melittin with glutamic acid substituting for all basic residues, and antimicrobial peptide bombolitin V with glutamic acid substituting for all basic residues.

111. The immunogenic nanoparticle according to any one of claims 1-110, wherein said nanoparticle contains a compound that increases immunogenicity.

112. The immunogenic nanoparticle of claim 111, wherein said compound is a compound that permits or induce the maturation of dendritic cells (DCs).

113. The immunogenic nanoparticle of claim 112, wherein said compound that permit or induce the maturation of dendritic cells (DCs) is selected from the group consisting of a lipopolysaccharide, TNF-alpha, and a CD40 ligand.

114. The immunogenic nanoparticle of claim 113, wherein said compound is selected from the group consisting of IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IFN-alpha, IFN-beta, IFN-gamma, GM-CSF, G-CSF, M-CSF, LT-beta, TNF-alpha, and hGH.

115. The immunogenic nanoparticle of claim 113, wherein said compound is selected from a cytokine shown in Table 16.

116. The immunogenic nanoparticle according to any one of claims 1-115, wherein said immunogenic nanoparticle is effective to raise an immune response when administered to a mammal.

117. The immunogenic nanoparticle of claim 116, wherein said nanoparticle is effective to raise a humoral immune response when administered to a mammal.

118. The immunogenic nanoparticle according to any one of claims 116-117, wherein said nanoparticle is effective to raise a cellular immune response when administered to a mammal.

119. The immunogenic nanoparticle according to any one of claims 116-118, wherein said nanoparticle is effective to raise one or more panels of neutralizing antibodies directed against the virus from which the viral protein(s) and/or protein fragments(s) are derived when administered to a mammal.

120. The immunogenic nanoparticle according to any one of claims 116-119, wherein said nanoparticle is effective to raise a protective immune response against the virus from which the antigen(s) are derived when administered to a mammal.

121. The immunogenic nanoparticle of claim 120, wherein said protective immune response is partially protective.

122. The immunogenic nanoparticle of claim 120, wherein said protective immune response is fully protective.

123. The immunogenic nanoparticle according to any one of claims 116-122, wherein said mammal is a human.

124. The immunogenic nanoparticle according to any one of claims 116-122, wherein said mammal is a non-human mammal.

125. The immunogenic nanoparticle according to any one of claims 1-124, wherein said nanoparticle is not hollow.

126. The immunogenic nanoparticle according to any one of claims 1-125, wherein said biocompatible polymer nanoparticle has a molecular weight greater than 10 kDa, or greater than about 20 kDa, or greater than about 30 kDa.

127. The immunogenic nanoparticle of claim 126, wherein said biocompatible polymer has a molecular weight less than about 100 kDa, or less than about 90 kDa, or less than about 80 kDa, or less than about 70 kDa, or less than about 60 kDa.

128. The immunogenic nanoparticle according to any one of claims 126-127, wherein said biocompatible polymer has a molecular weight ranging from about 30 kDa to about 60 kDa, or from about 35 kDa to about 55 kDa, or is about 40 kDa.

129. The immunogenic nanoparticle according to any one of claims 1-128, wherein said viral proteins or fragment(s) thereof do not comprise or do not consist of MERS-CoV RBD.

130. The immunogenic nanoparticle according to any one of claims 1-129, wherein said viral proteins or fragment(s) thereof do not comprise or do not consist of SARS-CoV-2 RBD.

131. The immunogenic nanoparticle according to any one of claims 1-130, wherein said adjuvant does not comprise the STING agonist cdGMP.

132. A pharmaceutical formulation comprising:
a population of immunogenic nanoparticles according to any one of claims 1-131; and
a pharmaceutically acceptable carrier.

133. The pharmaceutical formulation of claim 132, wherein said formulation is formulated for administration via a route selected from the group consisting of subcutaneous administration, intramuscular administration, inhalation, topical microneedle administration, and oral administration.

134. A method of inducing an immune response directed against a viral protein or viral protein fragment in a mammal, said method comprising:
administering to said mammal an effective amount of a population of immunogenic nanoparticles according to any one of claims 1-131, and/or a pharmaceutical formulation according to any one of claims 132-133.

135. The method of claim 134, wherein said viral proteins or fragments thereof are derived from a virus selected from the group consisting of corona viruses (including, but not limited to 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), SARS-CoV-2 (the novel coronavirus that causes coronavirus disease 2019, or COVID-19), and the like), human influenza viruses Types A and B, human Rhinovirus, respiratory syncytial virus (e.g., sin-SISH-uhl), parainfluenza viruses (HPIVs), measles virus, rubella virus, chickenpox/shingles virus, smallpox, chikungunya virus, hepatitis viruses, herpes viruses, human papilloma viruses (HPVs), polio virus, Norovirus, Rotavirus, Marburg virus, Ebola virus, Rabies, Smallpox, HIV, Hantavirus, Dengue, Molluscum contagiosum, Herpes simplex virus-1 (HSV-1), and Varicella-zoster virus (VZV).

136. The method of claim 134, wherein said viral proteins or fragments thereof are derived from a virus weaponized or studied as an agent for biological warfare.

137. The method of claim 136, wherein said virus is selected from the group consisting of the Bunyaviridae (especially Rift Valley fever virus), Ebolavirus, many of the Flaviviridae (especially Japanese encephalitis virus), Machupo virus, Marburg virus, Variola virus, yellow fever virus, viral encephalitis alphaviruses (e.g., venezuelan equine encephalitis, eastern equine encephalitis, western equine encephalitis), hantaviruses, Nipah virus, Tickborne encephalitis viruses, tickborne hemorrhagic fever viruses, and Yellow fever.

138. The method of claim 135, wherein said coronavirus comprises a member of the Betacoronavirus Genus.

139. The method of claim 138, wherein said coronavirus comprises a coronavirus that produces severe acute respiratory syndrome (SARS).

140. The method of claim 139, said coronavirus is a coronavirus selected from the group consisting of that SARS-CoV-2 (2019-nCoV), SARS-CoV-2, and MERS-CoV.

141. The method of claim 140, wherein said coronavirus is SARS-CoV-2 (2019-nCoV).

142. The method according to any one of claims 134-141, wherein said nanoparticle is effective to raise a humoral immune response when administered to a mammal.

143. The method according to any one of claims 134-142, wherein said nanoparticle is effective to raise a cellular immune response when administered to a mammal.

144. The method according to any one of claims 134-143, wherein said nanoparticle is effective to raise a neutralizing or protective antibody population directed against the virus from which the viral protein(s) and/or protein fragments(s) are derived when administered to a mammal.

145. The method according to any one of claims 134-144, wherein said nanoparticle is effect to raise a protective immune response against the virus from which the antigen(s) are derived when administered to a mammal.

146. A method for the prophylaxis and/or treatment of a viral infection in a mammal, said method comprising:
administering to said mammal an effective amount of a population of immunogenic nanoparticle according to any one of claims 1-124, and/or a pharmaceutical formulation according to any one of claims 132-133.

147. The method of claim 146, wherein said viral infection is produced by a virus selected from the group consisting of corona viruses (including, but not limited to 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), SARS-CoV-2 (the novel coronavirus that causes coronavirus disease 2019, or COVID-19), and the like), human influenza viruses Types A and B, human Rhinovirus, respiratory syncytial virus (e.g., sin-SISH-uhl), parainfluenza viruses (HPIVs), measles virus, rubella virus, chickenpox/shingles virus, smallpox, chikungunya virus, hepatitis viruses, herpes viruses, human papilloma viruses (HPVs), polio virus, Norovirus, Rotavirus, Marburg virus, Ebola virus, Rabies, Smallpox, HIV, Hantavirus, Dengue, Molluscum contagiosum, Herpes simplex virus-1 (HSV-1), and Varicella-zoster virus (VZV).

148. The method of claim 146, wherein said viral infection is produced by a virus weaponized or studied as an agent for biological warfare.

149. The method of claim 148, wherein said virus is selected from the group consisting of the Bunyaviridae (especially Rift Valley fever virus), Ebolavirus, many of the Flaviviridae (especially Japanese encephalitis virus), Machupo virus, Marburg virus, Variola virus, yellow fever virus, viral encephalitis alphaviruses (e.g., venezuelan equine encephalitis, eastern equine encephalitis, western equine encephalitis), hantaviruses, Nipah virus, Tickborne encephalitis viruses, tickborne hemorrhagic fever viruses, and Yellow fever.

150. The method of claim 147, wherein said coronavirus comprise a member of the Betacoronavirus Genus.

151. The method of claim 150, wherein said coronavirus comprises a coronavirus that produces severe acute respiratory syndrome (SARS).

152. The method of claim 151, said coronavirus is a coronavirus selected from the group consisting of that SARS-CoV-2 (2019-nCoV), SARS-CoV-2, and MERS-CoV.

153. The method of claim 152, wherein said coronavirus is SARS-CoV-2 (2019-nCoV).

154. The method according to any one of claims 146-153, wherein said method is effective to raise a humoral immune response in said mammal.

155. The method according to any one of claims 146-154, wherein said method is effective to raise a cellular immune response in said mammal.

156. The method according to any one of claims 146-155, wherein said method is effective to raise neutralizing antibody population directed against the virus.

157. The method according to any one of claims 146-156, wherein said method is effective to raise a protective immune response against the virus.

158. The method of claim 157, wherein said protective immune response is partially protective.

159. The method of claim 157, wherein said protective immune response is fully protective.

160. The method according to any one of claims 134-159, wherein said mammal is a human.

161. The method according to any one of claims 134-159, wherein said mammal is a non-human mammal.
